(19) United States
(12) Patent Application Publication
(10) Pub. No.: US 2014/0142094 A1
(43) Pub. Date: May 22, 2014

(54) KINASE INHIBITORS

(71) Applicants: M.V. Ramana Reddy, Blue Bell, PA (US); E. Premkumar Reddy, Villanova, PA (US)

(72) Inventors: M.V. Ramana Reddy, Blue Bell, PA (US); E. Premkumar Reddy, Villanova, PA (US)

(73) Assignee: ICAHN SCHOOL OF MEDICINE AT MOUNT SINAI, New York, NY (US)

(21) Appl. No.: 14/114,518

(22) PCT Filed: Apr. 30, 2012
(86) PCT No.: PCT/US12/35880

§ 371 Date: Feb. 4, 2014

Related U.S. Application Data
(60) Provisional application No. 61/480,687, filed on Apr. 29, 2011.

(51) Int. Cl. C07D 513/04; C07D 239/48; C07D 403/12; C07D 239/56; C07D 239/58; C07D 239/47; C07D 401/12

(52) U.S. Cl. CPC C07D 513/04; C07D 239/47; C07D 239/48; C07D 401/12; C07D 239/56; C07D 239/58; C07D 403/12
USPC 514/224.2; 544/48; 544/319; 544/295; 544/311; 544/296; 544/122; 544/321

(57) ABSTRACT

This disclosure relates to compounds, methods for their preparation, pharmaceutical compositions including these compounds and methods for the treatment of cellular proliferative disorders, including, but not limited to cancer. The method includes administering to the patient a therapeutically effective amount of a compound of formula (I), or a pharmaceutically acceptable salt thereof.

### CROSS-REFERENCE TO RELATED APPLICATIONS

This application claims the benefit of U.S. Provisional Application No. 61/480, 687, filed on Apr. 29, 2011, which is hereby incorporated by reference in its entirety.

### TECHNICAL FIELD

This disclosure relates to compounds, methods for their preparation, pharmaceutical compositions including these compounds and methods for the treatment of cellular proliferative disorders, including, but not limited to, cancer.

### BACKGROUND

Cellular proliferative disorders are among the most common causes of death in developed countries. For diseases for which treatments exist, such as cancer, the existing treatments have undesirable side effects and limited efficacy. Identifying new effective drugs for cellular proliferative disorders, including cancer, is a continuing focus of medical research.

### SUMMARY

It has been found that certain compounds and compositions are kinase inhibitors and are useful for the treatment of cellular proliferative disorders including, but not limited to cancer. The compounds are useful, e.g., as pharmaceuticals.

The disclosure describes compounds of formula (I):

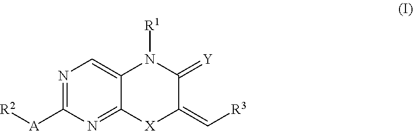

or a salt thereof, wherein:

A is selected from O, NR⁴ and S(O)ₘ;

R¹ is H or a substituted or unsubstituted (C₁-C₁₀)hydrocarbyl;

R² is selected from substituted or unsubstituted (C₁-C₁₀)hydrocarbyl and substituted or unsubstituted heterocyclyl;

R³ is selected from substituted or unsubstituted (C₆-C₁₀)aryl and substituted or unsubstituted (C₂-C₉)heteroaryl;

R⁴ is selected from H, (C₁-C₆)alkyl and —C(═O)R⁵;

or R² or R⁴ in combination with the nitrogen to which they are attached form a substituted or unsubstituted heterocyclyl;

R⁵ is selected from H and (C₁-C₆)alkyl;

X is S(O)ₙ;

Y is selected from O, S and NR⁶;

R⁶ is selected from H, —OH, (C₁-C₆)alkyl and —O—(C₁-C₆)alkyl;

m is an integer selected from 0, 1 and 2; and

n is an integer selected from 0, 1 and 2.

Also provided herein is a pharmaceutical composition, which comprises a compound of formula (I), or a pharmaceutically acceptable salt thereof, and a pharmaceutically acceptable carrier.

The disclosure also provides compounds that are useful as intermediates in the preparation of compounds of formula (I), and which may also be biologically active.

Further provided herein is a compound of formula (II):

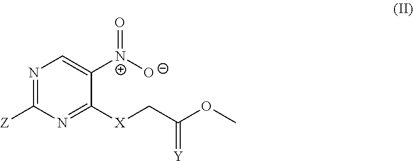

or a salt thereof, wherein:

X is S(O)ₙ;

Y is selected from O, S and NR⁶;

Z is a halogen; and

R⁶ is selected from hydrogen, —OH, (C₁-C₆)alkyl and —O—(C₁-C₆)alkyl;

n is an integer selected from 0, 1 and 2.

Provided herein is a compound of formula (III):

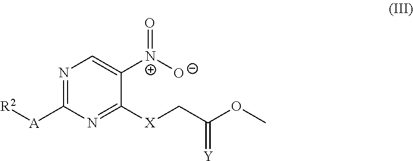

or a salt thereof, wherein:

A is selected from O, NR⁴ and S(O)ₘ;

R² is selected from substituted or unsubstituted (C₁-C₁₀)hydrocarbyl and substituted or unsubstituted heterocyclyl;

R⁴ is selected from H, (C₁-C₆)alkyl and —C(═O)R⁵;

or R² or R⁴ in combination with the nitrogen to which they are attached form a substituted or unsubstituted heterocyclyl;

R⁵ is selected from H and (C₁-C₆)alkyl;

X is S(O)ₙ;

Y is selected from O, S and NR⁶;

R⁶ is selected from H, —OH, (C₁-C₆)alkyl and —O—(C₁-C₆)alkyl;

m is an integer selected from 0, 1 and 2; and

n is an integer selected from 0, 1 and 2.

Further provided herein is a compound of formula (IV):

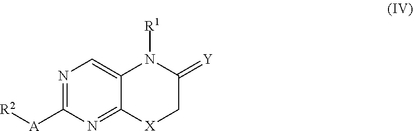

or a salt thereof, wherein:

A is selected from O, NR⁴ and S(O)ₘ;

R¹ is H or a substituted or unsubstituted (C₁-C₁₀)hydrocarbyl;

R² is selected from substituted or unsubstituted (C₁-C₁₀)hydrocarbyl and substituted or unsubstituted heterocyclyl;

R⁴ is selected from H, (C₁-C₆)alkyl and —C(═O)R⁵;

or R² or R⁴ in combination with the nitrogen to which they are attached form a substituted or unsubstituted heterocyclyl;

R⁵ is selected from H and (C₁-C₆)alkyl;

X is S(O)ₙ;

Y is selected from O, S and NR⁶;

R⁶ is selected from H, —OH, (C₁-C₆)alkyl and —O—(C₁-C₆)alkyl;

m is an integer selected from 0, 1 and 2; and

n is an integer selected from 0, 1 and 2.

A pharmaceutical composition comprising a compound of formula (IV), or a pharmaceutically acceptable salt thereof, and a pharmaceutically acceptable carrier is also provided herein.

A method for treating a cellular proliferative disorder in a patient is provided. The method includes administering to the patient a therapeutically effective amount of a compound of formula (I), or a pharmaceutically acceptable salt thereof.

A method of treating a neurological disorder in a patient is provided. The method includes administering to the patient a therapeutically effective amount of a compound of formula (I), or a pharmaceutically acceptable salt thereof.

A method of inhibiting one or more kinases in a patient is provided. The method includes administering to the patient a therapeutically effective amount of a compound of formula (I) or a pharmaceutically acceptable salt thereof.

A method of inhibiting one or more kinases in a cell is provided. The method includes contacting the cell with an effective amount of a compound of formula (I) or a pharmaceutically acceptable salt thereof.

A method of inhibiting cellular proliferation of cancer cells in a patient is provided. The method includes administering to the patient a therapeutically effective amount of a compound of formula (I) or a pharmaceutically acceptable salt thereof.

A method of inducing cell death of cancer cells in a patient is provided. The method includes administering to the patient a therapeutically effective amount of a compound of formula (I) or a pharmaceutically acceptable salt thereof.

A method of inducing apoptosis of cancer cells in a patient. The method includes administering to the patient a therapeutically effective amount of a compound of formula (I) or a pharmaceutically acceptable salt thereof.

A method of inducing apoptosis in a cell is provided. The method includes contacting the cell with an effective amount of a compound of formula (I) or a pharmaceutically acceptable salt thereof.

Methods of preparing compounds of Formulae (I), (II), (III) and (IV) are provided. The methods include one or more of the following steps:

(a) A compound of formula (II) can be prepared by reacting a compound of formula (VII):

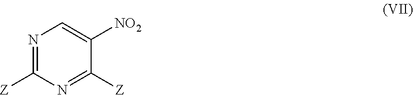

or a salt thereof, wherein each Z is a halogen, with a compound of formula (VIII):

HXCH₂CO₂CH₃ (VIII)

or a salt thereof, to prepare the compound of formula (II):

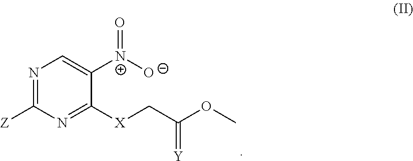

(b) A compound of formula (III) can be prepared by reacting a compound of formula (II):

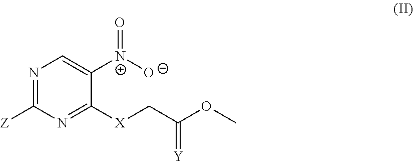

or a salt thereof, wherein Z is a halogen with a compound of formula (V):

R²-AH (V)

or a salt thereof, to form a compound of formula (III):

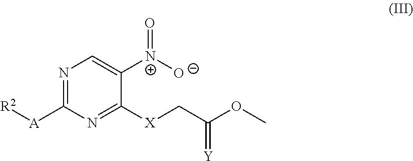

or a salt thereof.

(c) A compound of formula (IV) can be prepared by reducing a compound of formula (III):

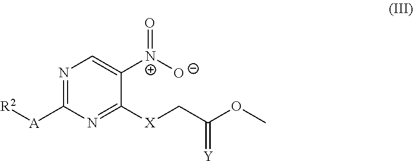

or a salt thereof, to produce the compound of formula (IV):

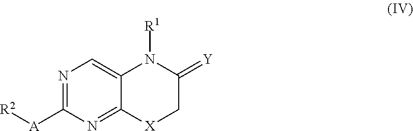

or a salt thereof.

(d) A compound of formula (I) can be prepared by reacting a compound of formula (IV):

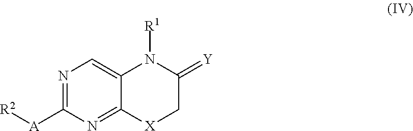

or a salt thereof.

with a compound of formula (VI):

R—C(═O)H (VI)

or a salt thereof, to form the compound according to formula (I):

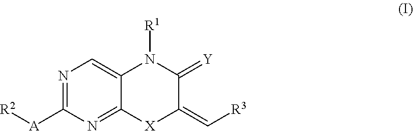

or a salt thereof.

The details of one or more embodiments of the invention are set forth in the accompanying drawings and the description below. Other features, objects, and advantages of the invention will be apparent from the description and drawings, and from the claims.

### DETAILED DESCRIPTION

It is appreciated that certain features described herein, which are, for clarity, described in the context of separate embodiments, can also be provided in combination in a single embodiment. Conversely, various features described herein which are, for brevity, described in the context of a single embodiment, can also be provided separately or in any suitable subcombination.

### I. DEFINITIONS

Unless defined otherwise, all technical and scientific terms used herein have the same meaning as is commonly understood by one of ordinary skill in the art to which this disclosure belongs. All patents, applications, published applications and other publications cited herein are incorporated by reference in their entirety. In the event that there is a plurality of definitions for terms cited herein, those in this section prevail unless otherwise stated.

For the terms “e.g.,” and “such as,” and grammatical equivalents thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise.

As used herein, the singular forms “a,” “an,” and “the” include plural referents unless the context clearly dictates otherwise.

The term “salt” includes any ionic form of a compound and one or more counter-ionic species (cations and/or anions). Salts also include zwitterionic compounds (i.e., a molecule containing one more cationic and anionic species, e.g., zwitterionic amino acids). Counter ions present in a salt can include any cationic, anionic, or zwitterionic species. Exemplary anions include, but are not limited to, chloride, bromide, iodide, nitrate, sulfate, bisulfate, sulfite, bisulfite, phosphate, acid phosphate, perchlorate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzenesulfonate, p-toluenesulfonate, p-trifluoromethylbenzenesulfonate, hydroxide, aluminates and borates. Exemplary cations include, but are not limited, to monovalent alkali metal cations, such as lithium, sodium, potassium and cesium, and divalent alkaline earth metals, such as beryllium, magnesium, calcium, strontium and barium. Also included are transition metal cations, such as gold, silver, copper and zinc, as well as non-metal cations, such as ammonium salts. The term “pharmaceutically-acceptable salt” refers to salts which possess toxicity profiles within a range that affords utility in pharmaceutical applications. Pharmaceutically unacceptable salts may nonetheless possess properties such as high crystallinity, which may render them useful, e.g., in processes of synthesis, purification or formulation of compounds described herein. In general the useful properties of the compounds described herein do not depend on whether the compound is or is not in a salt form, so unless clearly indicated otherwise (such as specifying that the compound should be in “free base” or “free acid” form), reference in the specification to a compound should be understood as including salt forms of the compound, whether or not this is explicitly stated. Preparation and selection of suitable salt forms is described in Handbook of Pharmaceutical Salts Properties, Selection, and Use By P. H. Stahl and C. G. Wermuth (Wiley-VCH 2002).

When in the solid state, the compounds described herein and salts thereof may occur in various forms and may, e.g., take the form of solvates, including hydrates. In general, the useful properties of the compounds described herein do not depend on whether the compound or salt thereof is or is in a particular solid state form, such as a polymorph or solvate, so unless clearly indicated otherwise reference in the specification to compounds and salts should be understood as encompassing any solid state form of the compound, whether or not this is explicitly stated.

Compounds provided herein can also include all isotopes of atoms occurring in the intermediates or final compounds. Isotopes include those atoms having the same atomic number but different mass numbers. For example, isotopes of hydrogen include tritium and deuterium.

The term “compound” as used herein is meant to include all stereoisomers, geometric isomers, tautomers and isotopes of the structures depicted. Compounds herein identified by name or structure as one particular tautomeric form are intended to include other tautomeric forms unless otherwise specified.

The term “hydrocarbyl” as used herein is meant to include a saturated or unsaturated, straight or branched, cyclic or acyclic, chiral or achiral organic compound or radical consisting of the elements carbon and hydrogen. These moieties include alkyl, alkenyl, alkynyl, cycloalkyl and aryl moieties. Unless otherwise indicated, these moieties preferably comprise 1 to 10 carbon atoms. In some embodiments, a hydrocarbyl moiety described herein includes hydrocarbyl moieties which are substituted with at least one atom other than carbon, including moieties in which a carbon chain atom is substituted with a hetero atom such as nitrogen, oxygen and sulfur.

Unless otherwise indicated, the alkyl groups described herein are lower alkyls containing from one to six carbon atoms in the principal chain. The term “(Cₓ-C_{y})alkyl” (wherein x and y are integers) by itself or as part of another substituent means, unless otherwise stated, an alkyl group containing from x to y carbon atoms. An alkyl group formally corresponds to an alkane with one C—H bond replaced by the point of attachment of the alkyl group to the remainder of the compound. An alkyl group may be straight-chained or branched. Examples of straight-chained alkyl groups include methyl, ethyl, n-propyl and n-butyl. Examples of branched alkyl groups include i-propyl, t-butyl and 2,2-dimethylethyl. (Cₓ-C_{y})alkyl groups include (C₁-C₆)alkyl and (C₁-C₃)alkyl, e.g., methyl and ethyl.

The term “(Cₓ-C_{y})alkylene” (wherein x and y are integers) refers to an alkylene group containing from x to y carbon atoms. An alkylene group formally corresponds to an alkane with two C—H bond replaced by points of attachment of the alkylene group to the remainder of the compound. Examples are divalent straight hydrocarbon groups consisting of methylene groups, such as, —CH₂—, —CH₂CH₂—, —CH₂CH₂CH₂—. The (Cₓ-C_{y})alkylene groups include (C₁-C₆)alkylene and (C₁-C₃)alkylene.

Unless otherwise indicated, the alkenyl groups described herein are lower alkenyls containing from two to six carbon atoms in the principal chain. They may be a straight or branched chain and include, e.g., ethenyl, propenyl, isopropenyl, butenyl, isobutenyl, hexenyl and the like. The term “(Cₓ-C_{y}) alkenyl” (wherein x and y are integers) denotes a radical containing x to y carbons, wherein at least one carbon-carbon double bond is present (therefore x must be at least 2). Some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixtures of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl and the like.

Unless otherwise indicated, the alkynyl groups described herein are lower alkynyls is containing from two to six carbon atoms in the principal chain. They may be a straight or branched chain and include, e.g., ethynyl, propynyl, butynyl, isobutynyl, hexynyl, and the like. The term “(Cₓ-C_{y}) alkynyl” (wherein x and y are integers) denotes a radical containing x to y carbons, e.g., 2 to 6 carbons, and at least one carbon-carbon triple bond, some embodiments are 2 to 4 carbons, some embodiments are 2 to 3 carbons, and some embodiments are 2 carbons. Examples of an alkynyl include ethynyl, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “cycloalkyl” as used herein describes a fully saturated or unsaturated, monocyclic or bicyclic alkyl ring system containing 3 to 12 carbons in the ring, such as cyclopentyl, cyclohexyl, cyclohexylmethyl, 4-methylcyclohexyl, bicyclo[2.2.1]heptanyl, norbornyl and adamantyl. The term “(Cₓ-C_{y}) cycloalkyl” (wherein x and y are integers) denotes a cycloalkyl group containing from x to y carbon atoms in the ring. Cycloalkyl groups having 7 or more carbon atoms may contain more than one ring and be polycyclic.

The term “aromatic” refers to a carbocycle or heterocycle having one or more polyunsaturated rings having aromatic character (i.e. having (4n+2) delocalized π (pi) electrons where n is an integer).

The term “aryl” as used herein, employed alone or in combination with other terms denotes an aromatic ring system composed of monocyclic or bicyclic rings and containing from 6 to 12 carbons in the ring, such as phenyl, biphenyl and naphthyl.

The terms “halogen” or “halo” as used herein refer to chlorine, bromine, fluorine and iodine.

The term “haloalkyl” as used herein refers to an alkyl group in which one or more of the hydrogen atoms has been replaced by a halogen atom. The term “(Cₓ-C_{y})haloalkyl” (wherein x and y are integers) by itself or as part of another substituent means, unless otherwise stated, an alkyl group containing from x to y carbon atoms. The alkyl may be substituted with one halogen up to fully substituted e.g. as represented by the formula CₙF₂ₙ₊₁; when more than one halogen is present they may be the same or different and selected from F, Cl, Br or I. Some embodiments are 1 to 3 carbons. Haloalkyl groups may be straight-chained or branched. Examples include fluoromethyl, difluoromethyl, trifluoromethyl, chlorodifluoromethyl, 2,2,2-trifluoroethyl, pentafluoroethyl and the like. The term “perfluoroalkyl” denotes the group of the formula —CₙF₂ₙ₊₁; stated differently, a perfluoroalkyl is an alkyl as defined herein wherein the alkyl is fully substituted with fluorine atoms and is therefore considered a subset of haloalkyl. Examples of perfluoroalkyls include CF₃, CF₂CF₃, CF₂CF₂CF₃, CF(CF₃)₂, CF₂CF₂CF₂CF₃, CF₂CF(CF₃)₂, CF(CF₃)CF₂CF₃ and the like.

The terms “heterocyclyl” or “heterocycle” or “heterocyclic” as used herein denotes a fully saturated or unsaturated, monocyclic or bicyclic group having at least one heteroatom in at least one ring, and 2 to 9 carbon atoms in the ring system. The heterocyclyl group has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclyl groups include oxirane, azetidine, pyrrolidine, pyrroline, imidazoline, pyrazolidine, dioxolane, tetrahydrofuran, piperidine and morpholine. Heterocyclyl rings can be aromatic (heteroaryl) or non-aromatic. The heteroatoms of the heterocyclyl ring system can include heteroatoms selected from one or more of nitrogen, oxygen and sulfur.

The term “heteroaryl” or “heteroaromatic” as used herein refers to an aromatic ring system having at least one heteroatom in at least one ring, and from 2 to 9 carbon atoms in the ring system. The heteroaryl group has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaryls include furyl, thienyl, pyridyl, oxazolyl, pyrrolyl, indolyl, quinolinyl, or isoquinolinyl and the like. The heteroatoms of the heteroaryl ring system can include heteroatoms selected from one or more of nitrogen, oxygen and sulfur.

Examples of non-aromatic heterocycles include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include: pyridyl, pyrazinyl, pyrimidinyl, particularly 2- and 4-pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,5-naphthyridinyl, 1,8-naphthyridinyl, 1,4-benzodioxanyl, coumarin, dihydrocoumarin, benzofuryl, particularly 3-, 4-, 5-, 6- and 7-benzofuryl, 2,3-dihydrobenzofuryl, 1,2-benzisoxazolyl, benzothienyl, particularly 3-, 4-, 5-, 6- and 7-benzothienyl, benzoxazolyl, benzthiazolyl, particularly 2-benzothiazolyl and 5-benzothiazolyl, purinyl, benzimidazolyl, particularly 2-benzimidazolyl and benztriazolyl.

The aforementioned listing of heterocyclyl and heteroaryl moieties is intended to be representative and not limiting.

The term “substituted” means that an atom or group of atoms formally replaces hydrogen as a “substituent” attached to another group. The term “substituted”, unless otherwise indicated, refers to any level of substitution, namely mono-, di-, tri-, tetra-, or penta-substitution, where such substitution is permitted. The substituents are independently selected, and substitution may be at any chemically accessible position. When groups are described herein as being substituted, the substituents can include, but are not limited to, (C₁-C₆)alkyl, (C₂-C₆)alkenyl, (C₂-C₆)alkynyl, halogen, (C₁-C₆)haloalkyl, —CN, —NO₂, —C(═O)R, —OC(═O)Ar, —C(═O)OR, —C(═O)NR^{E}₂, —C(═NR)NR₂, —OR, —Ar, —OAr, —((C₁-C₆)alkylene)Ar, —O((C₁-C₆)alkylene)Ar, —OC(═O)(C₁-C₆)alkyl, —OC(═O)O(C₁-C₆)alkyl, —OC(O)NR₂, —NR₂, —NRAr, —NR((C₁-C₆)alkylene)Ar, —NRC(═O)R, —NRC(═O)Ar, —C(═O)O(C₁-C₆)alkyl, —NRC(═O)NR₂, —NRSO₂R, —SR, —S(O)R, —SO₂R, —OSO₂(C₁-C₆)alkyl, —SO₂NR₂, (C₁-C₈)perfluoroalkyl, —(C₂-C₆)alkylene-OR, —O(C₂-C₆)alkylene-N((C₁-C₆)alkyl)₂, —P(═O)(OR)₂, —OP(═O)(OR)₂, wherein each R group is hydrogen or (C₁-C₆ alkyl), e.g., methyl and wherein each Ar is independently unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R, —C(═O)OR, —C(═O)NR₂, —C(═NR)NR₂, —OR, —OC(═O)(C₁-C₆)alkyl, —OC(═O)O(C₁-C₆)alkyl, —OC(O)NR₂, —NR₂, —NRC(═O)R, —NRC(═O)O(C₁-C₆)alkyl, —NRC(═O)NR₂, —NRSO₂R, —SR, —S(O)R, —SO₂R, —OSO₂(C₁-C₆)alkyl, —SO₂NR₂, (C₁-C₈)perfluoroalkyl, —(C₂-C₆)alkylene-OR, —O(C₂-C₆)alkylene-N((C₁-C₆)alkyl)₂, —P(═O)(OR)₂, —OP(═O)(OR)₂ wherein each R group is hydrogen or (C₁-C₆ alkyl).

All compounds and pharmaceutically acceptable salts thereof, can be found together with other substances such as water and solvents (e.g. hydrates and solvates).

In some embodiments, the compounds provided herein, or salts thereof, are substantially isolated. By “substantially isolated” is meant that the compound is at least partially or substantially separated from the environment in which it was formed or detected. Partial separation can include, e.g., a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds, or salt thereof. Methods for isolating compounds and their salts are routine in the art.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds described herein include the conventional non-toxic salts of the parent compound formed, e.g., from non-toxic inorganic or organic acids. The pharmaceutically acceptable salts of the compounds described herein can be synthesized from a parent compound which contains a is basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. Conventional methods for preparing salt forms are described, e.g., in Handbook of Pharmaceutical Salts: Properties, Selection, and Use, Wiley-VCH, 2002.

The term “contacting” means bringing at least two moieties together, whether in an in vitro system or an in vivo system.

The expression “therapeutically effective amount,” when used to describe an amount of compound administered in a method, refers to the amount of a compound that achieves the desired pharmacological effect or other effect, e.g., an amount that inhibits the abnormal growth or proliferation, or induces apoptosis of cancer cells, resulting in a useful effect.

The terms “treating” and “treatment” mean causing a therapeutically beneficial effect, such as ameliorating existing symptoms, preventing additional symptoms, ameliorating or preventing the underlying metabolic causes of symptoms, postponing or preventing the further development of a disorder, and/or reducing the severity of symptoms that will or are expected to a develop.

As used herein, “patient” (as in the subject of the treatment) includes both mammals and non-mammals. Mammals include, e.g., humans; non-human primates, e.g. apes and monkeys; cattle; horses; sheep; rats; mice; dogs; cats; pigs; and goats. Non-mammals include, e.g., fish and birds.

### II. NOVEL COMPOUNDS

### A. Compounds of Formula (I):

This disclosure provides compounds of formula (I):

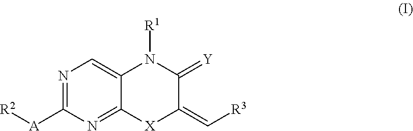

or a salt thereof, wherein:

A is selected from O, NR⁴ and S(O)ₘ;

R¹ is H or a substituted or unsubstituted (C₁-C₁₀)hydrocarbyl;

R² is selected from substituted or unsubstituted (C₁-C₁₀)hydrocarbyl and substituted or unsubstituted heterocyclyl;

R³ is selected from substituted or unsubstituted (C₆-C₁₀)aryl and substituted or unsubstituted (C₂-C₉)heteroaryl;

R⁴ is selected from H, (C₁-C₆)alkyl and —C(═O)R⁵;

or R² or R in combination with the nitrogen to which they are attached form a substituted or unsubstituted heterocyclyl;

R⁵ is selected from H and (C₁-C₆)alkyl;

X is S(O)ₙ;

Y is selected from O, S and NR⁶;

R⁶ is selected from H, —OH, (C₁-C₆)alkyl and —O—(C₁-C₆)alkyl;

m is an integer selected from 0, 1 and 2;

n is an integer selected from 0, 1 and 2.

In some embodiments of the compounds of formula (I), R¹ is selected from H, (C₁-C₆)alkyl, (C₂-C₆)alkenyl, (C₂-C₆)alkynyl and (C₃-C₇)cycloalkyl. For example, R¹ can be H. In some embodiments, R¹ is substituted. For example, R¹ can be substituted one or more substituents selected from halogen, —OR^{a1}, (CH₂)_{q1}OR^{a1}, —SR^{a1}, —NO₂, —NR^{a1}R^{b1}, —CN, (C₁-C₆)hydrocarbyl, (C₁-C₆)haloalkyl, —C(═O)R^{a1}, —C(═O)OR^{a1}, —C(═O)NR^{a1}R^{b1}, —C(═NR^{a1})NR^{a1}₂, —OC(═O)R^{a1}, —OC(═O)OR^{a1}, —OC(═O)NR^{a1}₂, —O—(CH₂)_{q1}OR^{a1}, —O—(CH₂)_{q1}NR^{a1}R^{b1}, —O—(CH₂)_{q1}-halo, —NR^{a1}C(═O)R^{a1}, —NR^{a1}C(O)OR^{a1}, —NR^{a1}C(═O)NR^{a1}₂, —NR^{a1}SO₂R^{a1}, —S(O)R^{a1}, —SO₂R^{a1}, —O—SO₃R^{a1}, —O—SO₂R^{a1}, —SO₂NR^{a1}₂, —O—P(═O)(OR^{a1})₂, —P(═O)(OR^{a1})₂, 4-methylpiperazin-1-yl, 4-BOC-piperazin-1-yl, 4-acetylpiperazin-1-yl, —O-glycoside and —O-glucuronide; wherein each q1 is an integer independently selected from 2, 3 and 4, and R^{a1} and R^{b1} are independently selected from H and (C₁-C₆)hydrocarbyl, e.g., alkyl, or R^{a1} and R^{b1} in any NR^{a1}R^{b1} group optionally together with the nitrogen to which they are attached form a heterocyclic ring, e.g., a 5, 6, or 7-membered ring, e.g., wherein R^{a1} and R^{b1} together form —(CH₂)₁₋₃-Q¹-(CH₂)₁₋₃—, where Q¹ is selected from a bond, CH₂, O, S and NR^{e}, wherein R^{c} is H or a (C₁-C₆)hydrocarbyl, e.g., (C₁-C₆)alkyl.

In some embodiments of the compounds of formula (I), R¹ when substituted, may be substituted with one or more substituents independently selected from (C₁-C₆)alkyl, (C₂-C₆)alkenyl, (C₂-C₆)alkynyl, halogen, —CN, —NO₂, —C(═O)R^{a1}, —C(═O)OR^{a1}, —C(═O)NR^{a1}₂, —C(═NR^{a1})NR^{a1}₂, —OR^{a1}, —OC(═O)(C₁-C₆)alkyl, (C₁-C₆)haloalkyl, —OC(═O)O(C₁-C₆)alkyl, —OC(═O)NR^{a1}₂, —NR^{a1}₂, —NR^{a1}C(═O)R^{a1}, —NR^{a1}C(═O)O(C₁-C₆)alkyl, —NR^{a1}C(═O)NR^{a1}₂, —NR^{a1}SO₂R^{a1}, —SR^{a1}, —S(O)R^{a1}, —SO₂R^{a1}, —OSO₂(C₁-C₆)alkyl, —SO₂NR^{a1}₂, (C₂-C₉)heterocyclyl, (C₁-C₆)perfluoroalkyl (e.g., —CF₃), (C₂-C₆)alkylene-OR^{a1}, —O(C₂-C₆)alkylene-N((C₁-C₆)alkyl)₂, —P(═O)(OR^{b1})₂, —OP(═O)(OR^{b1})₂, 4-methylpiperazin-1-yl, 4-BOC-piperazin-1-yl and 4-acetylpiperazin-1-yl, wherein R^{a1} and R^{b1} are independently selected from H and (C₁-C₆)alkyl.

In some embodiments of the compounds of formula (I), R¹ is (C₁-C₆)alkyl.

In some embodiments of the compounds of formula (I), R¹ is H.

In some embodiments of the compounds of formula (I), R² can be selected from (C₁-C₆)alkyl, (C₃-C₇)cycloalkyl, substituted or unsubstituted —(CH₂)ᵣ₂(C₆-C₁₀)aryl, substituted or unsubstituted —(CH₂)ᵣ₂(C₂-C₇)heterocyclyl, e.g., heteroaryl, substituted or unsubstituted (C₆-C₁₀)aryl and substituted or unsubstituted (C₂-C₉)heterocyclyl, e.g., heteroaryl, wherein r2 is an integer selected from 1, 2, 3 and 4. In some embodiments, R² is a substituted (C₆-C₁₀)aryl. For example, R² can be a substituted C₆ aryl (phenyl), such as a para-substituted C₆ aryl. Examples of R² groups include 4-(4-acetylpiperazin-1-yl)phenyl, 4-(4-methylpiperazin-1-yl)phenyl, 4-(4-methylpiperidin-1-yl)phenyl and 4-morpholinophenyl.

In some embodiments, R² is a substituted (C₂-C₉)heteroaryl. R² can be, e.g., a substituted C₂₋₅ monocyclic heteroaryl.

In some embodiments of the compounds of formula (I), R² is substituted. For example, R², when substituted, can be substituted with one or more substituents independently selected from halogen, —OR^{a2}, (CH₂)_{q2}OR^{a2}, —SR^{a2}, —NO₂, —NR^{a2}R^{b2}, —CN, (C₁-C₆)hydrocarbyl, (C₁-C₆)haloalkyl, —C(═O)R^{a2}, —C(═O)OR^{a2}, —C(O)NR^{a2}R^{2b}, —C(═NR^{a2})NR^{a2}₂, —OC(═O)R^{a2}, —OC(═O)OR^{a2}, —OC(═O)NR^{a2}₂, —O—(CH₂)_{q2}OR^{a2}, —O—(CH₂)_{q2}NR^{a2}R^{b2}, —O—(CH₂)_{q2}-halo, —NR^{a2}C(═O)R^{a2}, —NR^{a2}C(═O)OR^{a2}, —NR^{a2}C(═O)NR^{a2}₂, —NR^{a1}SO₂R^{a2}, —S(O)R^{a2}, —SO₂R^{a2}, —O—SO₃R^{a2}, —O—SO₂R^{a2}, —SO₂NR^{a2}, —O—P(═O)(OR^{a2})₂, —P(═O)(OR^{a2})₂, 4-methylpiperazin-1-yl, 4-BOC-piperazin-1-yl, 4-acetylpiperazin-1-yl, —O-glycoside and —O-glucuronide; wherein q2 is an integer selected from 2, 3 and 4, and R^{a2} and R^{b2} are independently selected from H and (C₁-C₆)hydrocarbyl, or R^{a2} and R^{b2} in any NR^{a2}R^{b2} group optionally together with the nitrogen to which they are attached form a heterocyclic ring, e.g., a 5, 6, or 7-membered ring, e.g., wherein R^{a2} and R^{b2} together form a group of the formula —(C₁-C₃)alkylene-Q²-(C₁-C₃)alkylene-. The ring formed by R^{a2} and R^{b2} and the nitrogen to which they are both attached may be a 5 to 7 membered ring. Q² is selected from a bond, —CH₂—, —CH((C₁-C₆)alkyl)-, e.g., —CHMe-, —C((C₁-C₆)alkyl)₂-, e.g., —CMe₂-, —CHAr^{Q2}—, —C((C₁-C₆)alkyl)Ar^{Q2}—, —O—, —S—, —NH—, —N((C₁-C₆)hydrocarbyl), —N((C₁-C₆)alkyl)-, e.g., —NMe-, —N(C(═O)(C₁-C₆)alkyl))-, e.g., —NAc—, —NAr^{Q2}— and —NC(═O)Ar^{Q2}—. In some embodiments, Q² is —NAr^{Q2}—. In some embodiments, Q² is —NC(═O)Ar^{Q2}—. In some embodiments, the —(C₁-C₃)alkylene-Q²-(C₁-C₃)alkylene- group is a group of the formula-CH₂)₁₋₃-Q²-(CH₂)₁₋₃—, e.g., —(CH₂)₂-Q²-(CH₂)₂—. The group may be a pyrrolidine, piperidine or piperazine, morpholine or thiomorpholine ring. Ar^{Q2} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{Q2}, —C(═O)OR^{Q2}, —C(═O)NR^{Q2}₂, —C(═NR^{Q2})NR^{Q2}₂, —OR^{Q2}, —OC(═O)(C₁-C₆)alkyl, —OC(═O)O(C₁-C₆)alkyl, —OC(═O)NR^{Q2}₂, —NR^{Q2}₂, —NR^{Q2}C(═O)R^{Q2}, —NR^{Q2}C(═O)O(C₁-C₆)alkyl, —NR^{Q2}C(═O)NR^{Q2}₂, —NR^{Q2}SO₂R^{Q2}, —SR^{Q2}, —S(O)R^{Q2}, —SO₂R^{Q2}, —OSO₂(C₁-C₆)alkyl, —SO₂NR^{Q2}₂,

(C₁-C₈)perfluoroalkyl, —(C₂-C₆)alkylene-OR^{Q2}, —O(C₂-C₆)alkylene-N((C₁-C₆)alkyl)₂, —P(═O)(OR²)₂ and —OP(═O)(OR²)₂; wherein each R^{Q2} is independently H or (C₁-C₆)alkyl. Ar^{Q2} may be a substituted or unsubstituted monocyclic aromatic ring, e.g., substituted or unsubstituted phenyl, or, e.g., (C₂-C₈)heteroaryl, e.g., pyridyl, pyrazinyl, pyrimidinyl or pyridazinyl. In some embodiments, when R² is substituted, it is monosubstituted or disubstituted, e.g., monosubstituted. When R² is a monocyclic aryl or heteroaryl ring, substituents may be attached at the 3- and/or at the 4-position relative to the point of attachment of the substituent to the remainder of the molecule. R² may be, e.g., a 3-substituted or a 4-substituted, or a 3,4-disubstituted aromatic ring (e.g., phenyl).

In some embodiments, R² is substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{a2}, —C(═O)OR^{a2}, —C(═O)NR^{a2}₂, —C(═NR^{a2})NR^{a2}₂, —OR^{a2}, —OC(═O)(C₁-C₆)alkyl, —OC(═O)O(C₁-C₆)alkyl, —OC(═O)NR^{a2}₂, —NR^{a2}₂, —NR^{a2}C(═O)R^{a2}, —NR^{a2}C(═O)O(C₁-C₆)alkyl, —NR^{a2}C(═O)NR^{a2}₂, —NR^{a2}SO₂R^{a2}, —SR^{a2}, —S(O)R^{a2}, —SO₂R¹², —OSO₂(C₁-C₆)alkyl, —SO₂NR^{a2}₂, (C₂-C₉)heterocyclyl, (C₁-C₆)perfluoroalkyl (e.g., —CF₃), (C₂-C₆)alkylene-OR, —O(C₂-C₆)alkylene-N((C₁-C₆)alkyl)₂, —P(═O)(OR^{a2})₂, —OP(═O)(OR^{b2})₂, 4-methylpiperazin-1-yl, 4-BOC-piperazin-1-yl and 4-acetylpiperazin-1-yl, wherein R^{a2} and R^{b2} are independently selected from H and (C₁-C₆)alkyl.

In some embodiments, R² is a group according to the following formula:

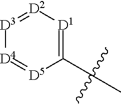

wherein:

D¹ is N or C-E¹;

D² is N or C-E²;

D³ is N or C-E³;

D⁴ is N or C-E⁴; and

D⁵ is N or C-E⁵;

provided that not more than three of D¹, D², D³, D⁴ and D⁵ is N.

E¹, E², E⁴ and E⁵ each independently selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(═O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂;

E³ is selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(═O)R⁷, —C(═O)NR⁷₂, —C(—NR¹)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NRC(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂; and a substituted or unsubstituted heterocyclic ring, linked via a nitrogen atom, e.g., a 5- to 7-membered ring, e.g., a pyrrolidine, piperidine, piperazine, morpholine or thiomorpholine ring; and

each R⁷ is independently selected from H and (C₁-C₆)alkyl.

In some embodiments, E³ is a pyrrolidine, piperidine or piperazine or morpholine ring substituted with one or more alkyl groups, e.g., 1, 2, 3, 4, or 5 alkyl groups. When E³ is a piperazine ring, the second nitrogen of the piperazine ring may be substituted with —C(═O)(C₁-C₆)alkyl, e.g., an acetyl group.

In some embodiments, E³ is a group according to the formula —NR⁸R⁹ wherein R⁸ and R⁹ in combination form a group according to the formula —(C₁-C₃)alkylene-Q^{E3}-(C₁-C₃)alkylene-. The ring formed by R⁸ and R⁹ and the nitrogen to which they are both attached may be a 5 to 7 membered ring. Q^{E3} is selected from a bond, —CH₂—, —CH((C₁-C₆)alkyl)-,

e.g., —CHMe-, —C((C₁-C₆)alkyl)₂-,
e.g., —CMe₂-, —CHAr^{E3}-, —C((C₁-C₆)alkyl)Ar^{E3}, —O—, —S—, —NH—, —N((C₁-C₆)alkyl)-,
e.g., —NMe-, —N(C(═O)(C₁-C₆)alkyl))-, e.g., —NAc—, —NAr^{E3}- and —NC(═O)Ar^{E3}-. In some embodiments, Q^{E3} is —NAr^{E3}-. In some embodiments, Q^{E3} is —NC(═O)Ar^{E3}-. In some embodiments, the —(C₁-C₃)alkylene-Q^{E3}-(C₁-C₃)alkylene- group is a group of the formula —(CH₂)₁₋₃-Q^{E3}-(CH₂)₁₋₃—, e.g., —(CH₂)₂-Q^{E3}-(CH₂)₂—. The group may be a pyrrolidine, piperidine or piperazine, morpholine or thiomorpholine ring. Are is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{E3}, —C(═O)OR^{E3}, —C(═O)NR^{E3}₂, —C(═NR^{E3})NR^{E3}₂, —OR^{E3}, —OC(═O)C₁-C₆)alkyl, —OC(═O)O(C₁-C₆)alkyl, —OC(═O)NR^{E3}₂, —NR^{E3}₂, —NR^{E3}C(═O)R^{E3}, —NR^{E3}C(═O)O(C₁-C₆)alkyl, —NR^{E3}C(═O)NR^{E3}₂, NR^{E3}SO₂R^{E3}, —SR^{E3}, —S(O)R^{E3}, —SO₂R^{E3}, —OSO₂(C₁-C₆)alkyl, —SO₂NR^{E3}₂, (C₁-C₈)perfluoroalkyl, —(C₂-C₆)alkylene-OR), —O(C₂-C₆)alkylene-N((C₁-C₆)alkyl)₂, —P(═O)(OR^{E3})₂ and —OP(═O)(OR^{E3})₂; wherein each R^{E3} is independently H or (C₁-C₆)alkyl. Ar^{E3} may be a substituted or unsubstituted monocyclic aromatic ring, e.g., substituted or unsubstituted phenyl, or, e.g., (C₂-C₅)heteroaryl, e.g., pyridyl, pyrazinyl, pyrimidinyl, or pyridazinyl.

In some embodiments, none of D¹, D², D³, D⁴ and D⁵ is N. In some embodiments, one of D¹, D², D³, D⁴ and D⁵ is N. In some embodiments, two of D¹, D², D³, D⁴ and D⁵ are N. In some embodiments, three of D¹, D², D³, D⁴ and D⁵ are N.

In some embodiments, D¹ is C-E¹, e.g., CH. In some embodiments, D¹ is N.

In some embodiments, D² is C-E², e.g., CH. In some embodiments, D² is N.

In some embodiments, D⁴ is C-E⁴, e.g., CH. In some embodiments, D⁵ is N.

In some embodiments, D⁵ is C-E⁵, e.g., CH. In some embodiments, D⁴ is N.

In some embodiments, D³ is N.

In some embodiments of the compounds of formula (I), A is O. In some embodiments, A is S(O)ₘ. In some such embodiments, A is S. In other such embodiments, A is S(O). In other such embodiments, A is S(O)₂.

In some embodiments of the compounds of formula (I), A is NR⁴. In some such embodiments, R⁴ is H. In some embodiments, when A is NR⁴, R⁴ can be (C₁-C₆)alkyl, e.g., methyl, or, e.g., ethyl.

In some embodiments, when A is NR⁴, R⁴ is —C(═O)R⁵. In some such embodiments, R⁵ is H. In some such embodiments, R⁵ is (C₁-C₆)alkyl, e.g., methyl, or, e.g., ethyl.

In some embodiments of the compounds of formula (I), when A is NR⁴, R² or R⁴ in combination with the nitrogen to which they are attached can form a substituted or unsubstituted heterocyclyl. R² or R⁴ in combination with the nitrogen to which they are attached can form, e.g., a 5 to 7 membered substituted or unsubstituted heterocyclyl, e.g., a non-aromatic 5 to 7 membered substituted or unsubstituted heterocyclyl. R² or R⁴ in combination can form, e.g., a group according to the formula —(C₁-C₃)alkylene-Q^{R4}-(C₁-C₃)alkylene. The ring formed by R² and R⁴ and the nitrogen to which they are both attached can be a 5-7 membered ring. Q^{R4} is selected from a bond, —CH₂—, —CH((C₁-C₆)alkyl)-, e.g., —CHMe-, —C((C₁-C₆)alkyl)₂-, e.g., —CMe₂-, —CHAr^{R4}—, e.g., —CHPh-, —C((C₁-C₆)alkyl)Ar^{R4}—, e.g., —CMePh-, —O—, —S—, —NH—, —N((C₁-C₆)alkyl)-, e.g., —NMe-, —NC(═O)((C₁-C₆)alkyl)-, e.g., —NAc—, —NAr^{R4}—, e.g., —NPh- and —NC(═O)Ar^{R4}—, e.g., —NC(═O)Ph-. In some embodiments, Q^{R4} is —NAr^{R4}—. In some embodiments, Q^{R4} is —NC(═O)Ar^{R4}-. In some embodiments, the —(C₁-C₃)alkylene-Q^{R4}-(C₁-C₃)alkylene- group is a group of the formula —(CH₂)₁₋₃-Q^{R4}-(CH₂)₁₋₃—, e.g., —(CH₂)₂-QR⁴—(CH₂)₂—. The group may be a pyrrolidine, piperidine, piperazine, morpholine or thiomorpholine ring. Ar^{R4} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R⁴, —C(═O)OR^{R4}, —C(═O)NR^{R4}₂, —C(═NR^{R4})NR^{R4}₂, —OR^{R4}, —OC(═O)(C₁-C₆)alkyl, —OC(═O)O(C₁-C₆)alkyl, —OC(═O)NR^{R4}₂, —NR^{R4}₂, —NR^{R4}C(═O)R^{R4}, —NR^{R4}C(═O)O(C₁-C₆)alkyl, —NR^{R4}C(═O)NR^{R4}₂, —NR^{R4}SO₂R^{R4}, —SR^{R4}, —S(O)R^{R4}, —SO₂R^{R4}, —OSO₂(C₁-C₆)alkyl, —SO₂NR^{R4}₂, (C₁-C₈)perfluoroalkyl, —(C₂-C₆)alkylene-OR^{R4}, —O(C₂-C₆)alkylene-N((C₁-C₆)alkyl)₂, —P(═O)(OR^{R4})₂ and —OP(═O)(OR^{R4})₂; wherein each R^{R4} is independently selected from H and (C₁-C₆)alkyl. Ar^{R4} may be a substituted or unsubstituted monocyclic aromatic ring, e.g., substituted or unsubstituted phenyl, or, e.g., (C₂-C₅)heteroaryl, e.g., a pyridyl, pyrazinyl, pyrimidinyl, or pyridazinyl ring.

Examples of —NR²R⁴ groups when A is NR⁴ and R² and R⁴ together form a ring include 4-(pyridin-2-yl)piperazin-1-yl and 4-(pyrimidin-2-yl)piperazin-1-yl.

In some embodiments, R³ is substituted or unsubstituted aryl. In some such embodiments, R³ may be phenyl. In some such embodiments, R¹ may be unsubstituted phenyl. In some such embodiments, R³ may be mono-, or di-, or tri-, or tetra- or penta-substituted phenyl. In some such embodiments, R³ may be 2-, 3-, 4-, 2,3-, 2,4-, 2,5-, 3,4-, 3,5-, 2,6-, 2,3,4-, 2,3,5-, 2,3,6-, 2,4,6-, 3,4,5-, 2,3,4,5-, 2,3,4,6-, 2,3,5,6- or 2,3,4,5,6-substituted phenyl. In some such embodiments, R¹ may be naphthyl. In some such embodiments, the substituents may be independently selected from (C₁-C₆)alkyl, (C₂-C₆)alkenyl, (C₂-C₆)alkynyl, halogen, (C₁-C₆)haloalkyl, —CN, —NO₂, —C(═O)^{R3}, —OC(═O)Ar^{R3}, —C(═O)OR^{R3}, —C(═O)NR^{R3}₂, —C(═NR^{R3})NR^{R3}₂, —OR^{R3}, —Ar^{R3}, —OAr^{R3}, —((C₁-C₆)alkylene)Ar^{R3}, —O((C₁-C₆)alkylene)Ar^{R3}, —OC(═O)(C₁-C₆)alkyl, —OC(═O)O(C₁-C₆)alkyl, —OC(═O)NR^{R3}₂, —NR^{R3}₂, —NR^{R3}Ar^{R3}, —NR^{R3}((C₁-C₆)alkylene)Ar^{R3}, —NR¹³C(═O)R^{R3}, —NR^{R3}C(═O)Ar^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl, —(C₂-C₆)alkylene-OR^{R3}, —O(C₂-C₆)alkylene-N((C₁-C₆)alkyl)₂, —P(═O)(OR^{R3})₂ and —OP(═O)(OR^{R3})₂; wherein each R^{R3} is independently selected from H and (C₁-C₆)alkyl; and wherein each Ar^{R3} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{R3}, —C(═O)OR^{R3}, —C(═O)NR^{R3}₂, —C(═NR^{R3})NR^{R3}₂, —OR^{R3}, —OC(═O)(C₁-C₆)alkyl, —OC(═O)O(C₁-C₆)alkyl, —OC(═O)NR^{R3}₂, —NR^{R3}₂, —NR^{R3}C(═O)R^{R3}, —NR^{R3}C(═O)O(C₁-C₆)alkyl, —NR^{R3}C(═O)NR^{R3}₂, —NR^{R2}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl and —(C₂-C₆)alkylene-OR^{R3}. In some such embodiments, the substituents may be selected from e.g., H, (C₁-C₆)alkyl, halogen, (C₁-C₆)haloalkyl, —CN, —NO₂, —C(═O)R^{R3}, —OC(═O)Ar^{R3}, —C(═O)OR^{R3}, —C(═O)NR^{R3}₂, —OR^{R3}, —Ar^{R3}, —OAr^{R3}, —O((C₁-C₆)alkylene)Ar^{R3}, —OC(═O)(C₁-C₆)alkyl, —NR^{R3}₂, —NR^{R3}Ar^{R3}, —NR^{R3}((C₁-C₆)alkylene)Ar^{R3}, —NR^{R3}C(═O)R^{R3} and —NR^{R3}C(═O)Ar^{R3}. In some such embodiments, the substituents may be selected from, e.g., H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph and. —NMeC(═O)Ph,

In some embodiments, R³ is selected from 4-acetoxyphenyl, 4-aminophenyl, 4-benzyloxyphenyl, 4-carboxyphenyl, 4-carbamoylphenyl, 4-cyanophenyl, 4-fluorophenyl, 4-hydroxyphenyl, 4-methoxy-3-nitrophenyl and 4-nitrophenyl.

In some embodiments, R³ is substituted or unsubstituted heteroaryl. In some such embodiments, R³ may a 5-membered heteroaryl ring, such as 2- or 3-furyl, 2- or 3-thiophenyl. In some such embodiments, R³ may a 6-membered heteroaryl ring such a pyridyl, pyrazinyl, pyrimidinyl, or pyridazinyl ring. R³ may a 9-membered heteroaryl ring such a benzofuran, benzothioazole, or indolyl ring, e.g., indole-3-yl. R³ may a 10-membered heteroaryl ring such a quinolyl or isoquinolyl ring. In some such embodiments, R³ may be mono-, or di-, or tri-, or tetra- or penta-substituted. In some such embodiments, the substituents may be independently selected from (C₁-C₆)alkyl, (C₂-C₆)alkenyl, (C₂-C₆)alkynyl, halogen, (C₁-C₆)haloalkyl, —CN, —NO₂, —C(═O)R^{R3}, —OC(═O)Ar^{R3}, —C(═O)OR^{R3}, —C(═O)NR^{R3}₂, —C(═NR^{R3})NR^{R3}₂, —OR^{R3}, —Ar^{R3}, —OAr^{R3}, —((C₁-C₆)alkylene)Ar^{R3}, —O((C₁-C₆)alkylene)Ar^{R3}, —OC(═O)(C₁-C₆)alkyl, —OC(═O)O(C₁-C₆)alkyl, —OC(═O)NR^{R3}₂, —NR^{R3}₂, —NR^{R3}Ar^{R3}, —NR^{R3}((C₁-C₆)alkylene)Ar^{R3}, —NR^{R2}C(═O)R^{R3}, —NR^{R3}C(═O)Ar^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R2}, —SO₂R^{R3}, —OSO₂(C₁-C₆)alkyl, —SO₂NR^{R3}₂, (C₁-C₈)perfluoroalkyl, —(C₂-C₆)alkylene-OR^{R3}, —O(C₂-C₆)alkylene-N((C₁-C₆)alkyl)₂, —P(═O)(OR^{R3})₂ and —OP(═O)(OR^{R3})₂; wherein each R^{R3} is independently selected from H and (C₁-C₆)alkyl; and wherein each Ar^{R3} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{R3}, —C(═O)OR^{R3}, —C(═O)NR^{R3}₂, —C(═NR^{R3})NR^{R3}₂, —OR^{R3}, —OC(═O)(C₁-C₆)alkyl, —OC(═O)O(C₁-C₆)alkyl, —OC(═O)NR^{R3}₂, —NR^{R3}₂, —NR^{R3}C(═O)R^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl and —(C₂-C₈)alkylene-OR^{R3}. In some such embodiments, the substituents may be selected from, e.g., H, (C₁-C₆)alkyl, halogen, (C₁-C₆)haloalkyl, —CN, —NO₂, —C(═O)R^{R3}, —OC(═O)Ar^{R3}, —C(═O)OR^{R3}, —C(═O)NR^{R3}₂, —OR^{R3}, —Ar^{R3}, —OAr^{R3}, —O((C₁-C₆)alkylene)Ar^{R3}, —OC(═O)(C₁-C₆)alkyl, —NR^{R3}₂, —NR^{R3}Ar^{R3}, —NR^{R3}((C₁-C₆)alkylene)Ar^{R3}, —NR^{R3}C(═O)R^{R3} and —NR^{R3}C(═O)Ar^{R3}. In some such embodiments, the substituents may be selected from, e.g., H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, NHC(═O)Me, —NMeC(═O)Me, —NHC(═O)Ph, or —NMeC(═O)Ph,

In some embodiments, R³ is 1-acetyl-1H-indol-3-yl.

In some embodiments, R³ is a group according to the following formula:

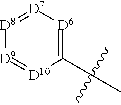

wherein

D⁶ is N or C-E⁶;

D⁷ is Nor C-E⁷;

D⁸ is Nor C-E⁸;

D⁹ is N or C-E⁹; and

D¹⁰ is N or C-E¹⁰;

provided that not more than three of D⁶, D⁷, D⁸, D⁹ and D¹⁰ is N.

E⁶ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E6}, —OC(═O)Ar^{E6}, —C(═O)OR^{E6}, —C(═O)NR^{E6}₂, —C(═NR^{E6})NR^{E6}₂, —OR^{E6}, —Ar^{E6}, —OAr^{E6}, —((C₁-C₆)alkylene)Ar^{E6}, —O((C₁-C₆)alkylene)Ar^{E6}, —OC(═O)(C₁-C₆)alkyl, —OC(═O)O(C₁-C₆)alkyl, —CO(═O)NR^{E6}₂, NR^{E6}₂, —NR^{E6}Ar^{E6}, —NR^{E6}((C₁-C₆)alkylene)Ar^{E6}, —NR^{E6}C(═O)R^{E6}, —NR^{E6}C(═O)Ar^{E6}, —NR^{E6}C(═O)O(C₁-C₆)alkyl, —NR^{E6}C(═O)NR^{E6}₂, —NR^{E6}SO₂R^{E6}, —SR^{E6}, —S(O)R^{E6}, —SO₂R^{E6}, —OSO₂(C₁-C₆)alkyl, —SO₂NR^{E6}₂, (C₁-C₈)perfluoroalkyl, —(C₂-C₆)alkylene-OR^{E6}, —O(C₂-C₆)alkylene-N((C₁-C₆)alkyl)₂, —P(═O)(OR^{E6})₂ and —OP(═O)(OR^{E6}); wherein each R^{E6} is independently selected from H and (C₁-C₆)alkyl; and wherein each Ar^{E6} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E6}, —C(═O)OR^{E6}, —C(═O)NR^{E6}₂, C(═R^{E6})NR^{E6}₂, —OR^{E6}, —OC(═O)(C₁-C₆)alkyl, —OC(═O)O(C₁-C₆)alkyl, —OC(═O)NR^{E6}₂, —NR^{E6}₂, —NR^{E6}C(═O)R^{E6}, —NR^{E6}C(═O)O(C₁-C₆)alkyl, NR^{E6}C(O)NR^{E6}₂, —NR^{E6}SO₂R^{E6}, —SR^{E6}, —S(O)R^{E6}, —SO₂R^{E6}, —OSO₂(C₁-C₆)alkyl, —SO₂NR^{E6}₂, (C₁-C₈)perfluoroalkyl and —(C₂-C₆)alkyene-OR^{E6};

E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —C(—NR⁷)NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R^{E7}, —NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N((C₁-C₆)alkyl)₂, —P(═O)(OR^{E7})₂ and —P(═O)(OR^{E7}); wherein each R^{E7} is independently selected from H and (C₁-C₆)alkyl; and wherein each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E7}, C(═O)OR^{E7}, —C(═O)NR^{E7}₂, —C(═NR^{E7})NR^{E7}₂, —OR^{E7}, —OC(═O)((C₁-C₆)alkyl, —OC(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)alkyl, N^{E7}C(═O)NR^{E7}₂, NR⁷SO₂R^{E7}, —SR^{E7}, —S(O)R^{E7}, —SO₂R^{E7}, —OSO₂(C₁-C₆)alkyl, —SO₂NR^{E7}₂, (C₁-C₆)perfluoroalkyl and —(C₂-C₆)alkylene —OR^{E7};

E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR, —C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, —NR^{E8}C(═O)(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl, —(C₂-C₆)alkylene-OR^{E8}, —O(C₂-C₆)alkylene-N((C₁-C₆)alkyl)₂, —P(═O)(OR^{E8})₂ and —OP(═O)(OR^{E8})₂; wherein each R^{E8} is independently selected from H and (C₁-C₆)alkyl; and wherein each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E8}, —C(═O)OR^{E8}, —C(═O)NR^{E8}₂, —C(═NR^{E8})NR^{E8}₂, —OR^{E8}, —OC(═O)(C₁-C₆)alkyl, —C(═O)O(C₁-C₆)alkyl, —OC(═O)NR^{E8}₂, —N^{E8}, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8};

E⁹ is selected from H, (C₁-C₆)alkyl, (C₂-C₆)alkenyl, (C₂-C₆)alkynyl, halogen, C(═O)NR^{E9}₂, —C(═NR^{E9})NR^{E9}₂, —OR^{E9}, —Ar^{E9}, —OAr^{E9}, —((C₁-C₆)alkylene)Ar^{E9}, —O((C₁-C₆)alkylene)Ar^{E9}, —OC(═O)(C₁-C₆)alkyl, —OC(═O)O(C₁-C₆)alkyl, —OC(═O)NR^{E9}₂, —NR^{E9}₂, —NR^{E9}Ar^{E9}, —NR^{E9}((C₁-C₆)alkylene)Ar^{E9}, —NR^{E9}C(═O)R^{E9}, —NR^{E9}C(═O)Ar^{E9}, —NR^{E9}C(═O)O(C₁-C₆)alkyl, —NR^{E9}C(═O)NR^{E9}₂, —NR^{E9}SO₂R^{E9}, —SR^{E9}, —S(O)R^{E9}, —SO₂R^{E9}, —OSO₂(C₁-C₆)alkyl, —SO₂NR^{E9}₂, (C₁-C₈)perfluoroalkyl, —(C₂-C₆)alkylene-OR^{E9}, —O(C₂-C₆)alkylene-N((C₁-C₆)alkyl)₂, —P(═O)(OR^{E9})₂ and —OP(═O)(OR⁹)₂; wherein each R^{E9} is independently selected from H and (C₁-C₆)alkyl; and wherein each Ar^{E9} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(O)R^{E9}, —C(═O)OR^{E9}, —(═O)NR^{E9}₂, —C(═NR^{E9})NR^{E9}₂, —OR^{E9}, —C(═O)O(C₁-C₆)alkyl, —OC(═O)O(C₁-C₆)alkyl, —OC(═O)NR^{E9}₂, —N^{E9}₂, —NR^{E9}C(═O)R^{E9}, —NR^{E9}C(═O)O(C₁-C₆)alkyl, —NR^{E9}C(═O)NR^{E9}₂, —NR⁷⁹SO₂R^{E9}, —SR^{E9}, —S(O)R^{E9}, —SO₂R^{E9}, —OSO₂(C₁-C₆)alkyl, —SO₂NR^{E9}₂, (C₁-C₈)perfluoroalkyl and —(C₂-C₆)alkylene OR^{E9}; and

E¹⁰ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E10}, —OC(═O)Ar^{E10}, —C(═O)R^{E10}, —C(═O)NR^{E10}₂, —C(═NR^{E10})NR^{E10}₂, —OR^{E10}, —Ar^{E10}, —OAr^{E10}, —((C₁-C₆)alkylene)Ar^{E10}, —O((C₁-C₆)alkylene)Ar^{E10}, —OC(═O)(C₁-C₆)alkyl, —OC(═O)O(C₁-C₆)alkyl, —OC(═O)NR^{E10}₂, —NR^{E10}₂, —NR^{E10}Ar^{E10}, —NR^{E10}((C₁-C₆)alkylene)Ar^{E10}, —NR^{E10}C(═O)R^{E10}, —NR^{E10}C(═O)Ar^{E10}, —NR^{E10}C(═O)O(C₁-C₆)alkyl, —NR^{E10}C(═O)NR^{E10}₂, —NR^{E10}SO₂R^{E10}, —SR^{E10}, —S(O)R^{E10}, —SO₂R^{E10}, —OSO₂(C₁-C₆)alkyl, —SO₂NR^{E10}₂, (C₁-C₈)perfluoroalkyl, —(C₂-C₆)alkylene-OR^{E10}, —O(C₂-C₆)alkylene-N((C₁-C₆)alkyl)₂, —P(═O)(OR^{E10})₂ and —OP(═O)(OR^{E10})₂; wherein each R^{E10} is independently selected from H and (C₁-C₆)alkyl; and wherein each Ar^{E10} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E10}, —C(═O)OR^{E10}, —C(═O)NR^{E10}₂, —C(═NR^{E10})NR^{E10}₂, —OR^{E10}, —OC(═O)(C₁-C₆)alkyl, —OC(═O)O(C₁-C₆)alkyl, —OC(═O)NR^{E10}₂, NR^{E10}₂, —NR^{E10}C(O)R^{E10}, —NR^{E10}C(═O)O(C₁-C₆)alkyl, —NR^{E10}C(═O)NR^{E10}₂, NR^{E10}SO₂R^{E10}, —SR^{E10}, —S(O)R^{E10}, —SO₂R^{E10}, —OSO₂(C₁-C₆)alkyl, —SO₂NR^{E10}₂, (C₁-C₈)perfluoroalkyl and —(C₂-C₆)alkylene-OR^{E10}.

Each of Ar^{E6}, Ar^{E7}, Ar^{E8}, Ar^{E9} and Ar^{E10} may be, e.g., substituted or unsubstituted phenyl.

In some embodiments, none of D⁶, D⁷, D⁸, D⁹ and D¹⁰ is N. In some embodiments, one of D⁶, D⁷, D⁸, D⁹ and D¹⁰ is N. In some embodiments, two of D⁶, D⁷, D⁸, D⁹ and D¹⁰ are N. In some embodiments, three of D⁶, D⁷, D⁸, D⁹ and D¹⁰ are N.

In some embodiments, D⁶ is C-E⁶, e.g., CH.

In some embodiments, D⁷ is C-E⁷. In some such embodiments, E⁷ may be, e.g., H, (C₁-C₆)alkyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —OR^{E7}, —Ar^{E7}, —OAr^{E7}, —O((C₁-C₆)alkylene)Ar^{E7}, —OC(═O)(C₁-C₆)alkyl, —NR^{E7}₂, —NR^{E7}Ar^{E7}, —NR^{E7}((C₁-C₆)alkylene)Ar^{E7}, —NR^{E7}C(═O)R^{E7}, or —NR^{E7}C(═O)Ar^{E7}. In some such embodiments, E⁷ may be, e.g., H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph, In some such embodiments, E⁷ may be, e.g., H or —NO₂.

In some embodiments, D⁸ is C-E⁸. In some such embodiments, E⁸ may be, e.g., H, (C₁-C₆)alkyl, halogen, (C₁-C₆)haloalkyl, —CN, —NO₂, —C(═O)R^{E8}, —OC(═O)Ar^{E8}, —C(═O)OR^{E8}, C(═O)NR^{E8}₂, —OR^{E8}, —Ar^{E8}, —OAr^{E8}, —O((C₁-C₆)alkylene)Ar^{E8}, —OC(═O)(C₁-C₆)alkyl, —NR^{E8}₂, —NR^{E8}Ar^{E8}, —NR^{E8}((C₁-C₆)alkylene)Ar^{E8}, —NR^{E8}C(═O)R^{E8}, or —NR^{E8}C(═O)Ar^{E8}. In some such embodiments, E⁸ may be, e.g., H, methyl, ethyl, —F, —Cl, —CN, —NO₂, —C(═O)Me, —OC(O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph,

In some embodiments, D⁹ is C-E⁹, e.g., CH.

In some embodiments, D¹⁰ is C-E¹⁰, e.g., CH.

In some embodiments, D⁶ is N. In some embodiments, D⁷ is N. In some embodiments, D⁸ is N. In some embodiments, D⁹ is N. In some embodiments, D¹⁰ is N.

In some embodiments of the compounds of formula (I), X is S.

In some embodiments of the compounds of formula (I), X is S(O).

In some embodiments of the compounds of formula (I), X is S(O)₂.

In some embodiments of the compounds of formula (I), Y is O.

In some embodiments of the compounds of formula (I), Y is S

In some embodiments of the compounds of formula (I), Y is NR⁶. In some such embodiments, R⁶ is H. In some such embodiments, R⁶ is —OH. In some such embodiments, R⁶ is (C₁-C₆)alkyl, e.g., methyl or ethyl. In some such embodiments, R⁶ is —O—(C₁-C₆)alkyl, e.g., methoxy or ethoxy.

The compounds of formula (I) can include compounds of formula (I-A):

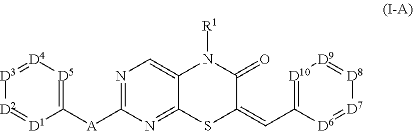

or a salt thereof, wherein:

A is selected from O, NR⁴ and S(O)ₘ;

R¹ is H or (C₁-C₆)alkyl;

D¹ is N or C-E¹;

D² is N or C-E²;

D³ is N or C-E³;

D⁴ is N or C-E⁴;

D⁵ is N or C-E⁵;

D⁶ is N or C-E⁶;

D⁷ is N or C-E⁷;

D⁸ is N or C-E⁸;

D⁹ is N or C-E⁹; and

D¹⁰ is N or C-E¹⁰;

provided that not more than three of D¹, D², D³, D⁴ and D⁵ is N; and

provided that not more than three of D⁶, D⁷, D⁸, D⁹ and D¹⁰ is N;

E¹, E², E⁴ and E⁵ each independently selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(═O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR, —O(C₂-C₆)alkylene-N((C₁-C₆)alkyl)₂, —P(═O)(OR⁷)₂ and —OP(═O)(OR⁷)₂;

E³ is selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(═O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NRC(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂; and a substituted or unsubstituted heterocyclic ring linked via a nitrogen atom, e.g., a 5- to 7-membered ring, e.g., a pyrrolidine, piperidine, piperazine, morpholine or thiomorpholine ring; and

each R⁷ is independently selected from H and (C₁-C₆)alkyl.

E⁶ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E6}, —OC(═O)Ar^{E6}, —C(═O)OR^{E6}, —C(═O)NR^{E6}₂, —C(═NR^{E6})NR^{E6}₂, —OR^{E6}, —Ar^{E6}, —OAr^{E6}, —((C₁-C₆)alkylene)Ar^{E6}, —O((C₁-C₆)alkylene)Ar^{E6}, —OC(═O)(C₁-C₆)alkyl, —OC(═O)O(C₁-C₆)alkyl, —OC(═O)NR^{E6}₂, NR^{E6}₂, —NR^{E6}Ar^{E6}, —NR^{E6}((C₁-C₆)alkylene)Ar^{E6}, —NR^{E6}C(═O)R^{E6}, —NR^{E6}C(═O)Ar^{E6}, —NR^{E6}C(═O)O(C₁-C₆)alkyl, —NR^{E6}C(═O)NR^{E6}₂, —NR^{E6}SO₂R^{E6}, —SR^{E6}, —S(O)R^{E6}, —SO₂R^{E6}, —OSO₂(C₁-C₆)alkyl, —SO₂NR^{E6}₂, (C₁-C₈)perfluoroalkyl, —(C₁-C₆)alkylene-OR^{E6}, —O(C₂-C₆)alkylene-N((C₁-C₆)alkyl)₂, —P(═O)(OR^{E6}) and —OP(═O)(OR^{E6})₂; wherein each R^{E6} is independently selected from H and (C₁-C₆)alkyl; and wherein each Ar^{E6} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E6}, —C(═O)OR^{E6}, —C(═O)NR^{E6}₂, —C(═N^{E6})NR^{E6}₂, —OR^{E6}, —OC(—O)(C₁-C₆)alkyl, —OC(═O)(C₁-C₆)alkyl, —OC(═O)NR^{E6}₂, —NR^{E6}₂, —NR^{E6}C(═O)R^{E6}, —NR^{E6}C(═O)O(C₁-C₆)alkyl, —NR^{E6}C(═O)NR^{E6}₂, —NR^{E6}SO₂R^{E6}, —SR^{E6}, —S(O)R^{E6}, —SO₂R^{E6}, —OSO₂(C₁-C₆)alkyl, —SO₂NR^{E6}₂, (C₁-C₈)perfluoroalkyl and —(C₂-C₆alkylene-OR^{E6};

E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(O)Ar^{E7}, —C(═O)OR^{E7}, —C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(—O)O(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R^{E7}, —NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N((C₁-C₆)alkyl)₂, —P(═O)(OR^{E7}) and —OP(═O)(OR^{E7})₂; wherein each R^{E7} is independently selected from H and (C₁-C₆)alkyl; and wherein each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E7}, —C(═O)OR^{E7}, C(═O)NR^{E7}₂, —C(═NR^{E7})NR^{E7}₂, —OR^{E7}, —OC(═O)(C₁-C₆)alkyl, —OC(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl and —(C₂-C₆)alkylene-OR^{E7};

E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, —NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —SO₂R^{E8}, —OSO₂(C₁-C₆)alkyl, —SO₂NR^{E8}₂, (C₁-C₈)perfluoroalkyl, —(C₂-C₆)alkylene-OR^{E8}, —O(C₂-C₆)alkylene-N((C₁-C₆)alkyl)₂, —P(═O)(OR^{E8})₂ and —OP(═O)(OR^{E8}); wherein each R^{E8} is independently selected from H and (C₁-C₆)alkyl; and wherein each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E8}, —C(═O)OR^{E8}, —C(═O)NR^{E8}₂, —C(═NR^{E8})NR^{E8}₂, —OR^{E8}, —OC(═O)(C₁-C₆)alkyl, —OC(═O)O(C₁-C₆)alkyl, —OC(═O)NR^{E8}₂, —NR^{E7}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8};

E⁹ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E9}, —OC(O)Ar^{E9}, —C(═O)OR^{E9}, —C(═O)NR E^{E9}₂, —C(═NR^{E9})NR^{E9}₂, —OR^{E9}, —Ar^{E9}, —OAr^{E9}, —((C₁-C₆)alkylene)Ar^{E9}, —O((C₁-C₆)alkylene)Ar^{E9}, —OC(═O)(C₁-C₆)alkyl, —OC(═O)O(C₁-C₆)alkyl, —OC(═O)NR^{E9}₂, —NR^{E9}₂, —NR^{E9}Ar^{E9}, —NR^{E9}((C₁-C₆)alkylene)Ar^{E9}, —NR^{E9}C(═O)R^{E9}, —NR^{E9}C(═O)Ar^{E9}, NR^{E9}C(═O)O(C₁-C₆)alkyl, —NR^{E9}C(═O)NR^{E9}₂, —NR^{E9}SO₂R^{E9}, —SR^{E9}, —S(O)R^{E9}, —SO₂R^{E9}, —OSO₂(C₁₋₆)alkyl, —SO₂NR^{E9}₂, (C₁-C₈)perfluoroalkyl, —(C₂-C₆)alkylene-OR^{E9}, —O(C₂-C₆)alkylene-N((C₁-C₆)alkyl)₂, —P(═O)(OR^{E9}) and —OP(═OR)(OR^{E9})₂; wherein each R^{E9} is independently selected from H and (C₁-C₆)alkyl; and wherein each Ar^{E9} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E9}, —C(═O)OR^{E9}, —C(═O)NR^{E9}₂, —C(═NR^{E9})NR^{E9}₂, —OR^{E9}, —OC(═O)(C₁-C₆)alkyl, —OC(═O)O(C₁-C₆)alkyl, —O(═O)NR^{E9}₂, —NR^{E9}₂, —NR^{E9}C(═O)R^{E9}, NR^{E9}C(═O)O(C₁-C₆)alkyl, —NR^{E9}C(═O)NR^{E9}₂, NR^{E9}SO₂R^{E9}, —SR^{E9}, —S(O)R^{E9}, —SO₂R^{E9}, —OSO₂(C₁-C₆)alkyl, —SO₂NR^{E9}₂, (C₁-C₈)perfluoroalkyl and —(C₂-C₆)alkylene-OR^{E9}; and

E¹⁰ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E10}, —OC(═O)Ar^{E10}, —C(═O)OR^{E10}, —C(═O)NR^{E10}₂, —C(═NR^{E10})NR^{E10}₂, —OR^{E10}, —Ar^{E10}, —OAr^{E10}, —((C₁-C₆)alkylene)Ar^{E10}, —O((C₁-C₆)alkylene)Ar^{E10}, —OC(═O)(C₁-C₆)alkyl, —OC(═O)O(C₁-C₆)alkyl, —OC(═O)NR^{E10}₂, —NR^{E10}₂, —NR^{E10}Ar^{E10}, —NR^{E10}((C₁-C₆alkylene)Ar^{E10}, —NR^{E10}C(═O)R^{E10}, —NR^{E10}C(═O)Ar^{E10}, —NR^{E10}C(═O)O(C₁-C₆)alkyl, —NR^{E10}C(═O)NR^{E10}₂, NR^{E10}SO₂R^{E10}, —SR^{E10}, —S(O)R^{E10}, —SO₂R^{E10}, —OSO₂(C₁-C₆)alkyl, —SO₂NR^{E10}₂, (C₁-C₁)perfluoroalkyl, —(C₂-C₆)alkylene-OR^{E10}, —O(C₂-C₆)alkylene-N((C₁-C₆)alkyl)₂, —P(═O)(OR^{E10})₂ and —OP(═O)(OR^{E10})₂; wherein each R^{E10} is independently selected from H and (C₁-C₆)alkyl; and wherein each Ar^{E10} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E10}, —C(═O)OR^{E10}, C(O)NR^{E10}₂, —C(═NR^{E10})NR^{E10}₂, —OR^{E10}, —OC(═O)(C₁-C₆)alkyl, —OC(═O)O(C₁-C₆)alkyl, —OC(═O)NR^{E10}₂, —NR^{E10}₂, —NR^{E10}C(═O)R^{E10}, —NR^{E10}C(═O)O(C₁-C₆)alkyl, —NR^{E10}C(═O)NR^{E10}₂, —NR^{E10}SO₂R¹⁰, —SR^{E10}, —S(O)R^{E10}, —SO₂R^{E10}, —OSO₂(C₁-C₆)alkyl, —SO₂NR^{E10}₂, (C₁-C₈)perfluoroalkyl and —(C₂-C₆)alkylene-OR^{E10}.

Each of Ar^{E6}, Ar^{E7}, Ar^{E8}, Ar^{E9} and Ar^{E10} may be, e.g., substituted or unsubstituted phenyl.

In some embodiments of the compounds of formula (I-A), R¹ is H.

In some embodiments of the compounds of formula (I-A), A is NR⁴, e.g., NH. In some embodiments, A is O.

In some embodiments of the compounds of formula (I-A), none of D¹, D², D³, D⁴ and D^{s} is N. In some embodiments, one of D¹, D², D³, D⁴ and D⁵ is N. In some embodiments, two of D¹, D², D³, D⁴ and D⁵ are N. In some embodiments, three of D¹, D², D³, D⁴ and D⁵ are N. In some embodiments, none of D⁶, D⁷, D⁸, D⁹ and D¹⁰ is N. In some embodiments, one of D⁶, D⁷, D⁸, D⁹ and D¹⁰ is N. In some embodiments, two of D⁶, D⁷, D⁸, D⁹ and D¹⁰ are N. In some embodiments, three of D⁶, D⁷, D⁸, D⁹ and D¹⁰ are N.

In some embodiments of the compounds of formula (I-A), D¹ is C-E¹, e.g., C—H. In some embodiments, D² is C-E², e.g., C—H. In some embodiments, D⁴ is C-E⁴, e.g., C—H. In some embodiments, D⁵ is C-E⁵, e.g., C—H. In some embodiments, D⁶ is C-E⁶, e.g., C—H. In some embodiments, D⁷ is C-E⁷, e.g., C—H. In some embodiments, D⁸ is C-E⁸, e.g., C—H. In some embodiments, D⁹ is C-E⁹, e.g., C—H. In some embodiments, D¹⁰ is C-E¹⁰, e.g., C—H. In some embodiments, D¹ is N. In some embodiments, D⁵ is N. In some embodiments, D⁶ is N. In some embodiments, D⁴ is N. In some embodiments, D⁵ is N. In some embodiments, D⁶ is N. In some embodiments, D⁷ is N. In some embodiments, D⁸ is N. In some embodiments, D⁹ is N. In some embodiments, D¹⁰ is N.

In some embodiments of the compounds of formula (I-A), D¹ is C—H; D² is C—H; D² is C—H; D⁵ is C—H; D⁶ is C—H; D⁹ is C—H; and D¹⁰ is C—H.

In some embodiments of the compounds of formula (I-A), D³ is C-E³. In some such embodiments, E³ is a pyrrolidine, piperidine or piperazine or morpholine ring substituted with one or more alkyl groups, e.g., 1, 2, 3, 4, or 5 alkyl groups. When E³ is a piperazine ring, the second nitrogen of the piperazine ring may be substituted with —C(═O)(C₁-C₆)alkyl, e.g., an acetyl group.

In some embodiments of the compounds of formula (I-A), E³ is a group according to the formula —NR⁸R⁹ wherein R⁸ and R⁹ in combination form a group according to the formula —(C₁-C₃)alkylene-Q^{E3}-(C₁-C₃)alkylene-. The ring formed by R⁸ and R⁹ and the nitrogen to which they are both attached may be a 5 to 7 membered ring. Q^{E3} is selected from a bond, —CH₂—, —CH((C₁-C₆)alkyl)-, e.g., —CHMe-, —C((C₁-C₆)alkyl)₂-, e.g., —CMe₂-, —CHAr^{E3}, —C((C₁-C₆)alkyl)Ar^{E3}—, —O—, —S—, —NH—, —N((C₁-C₆)alkyl)-, e.g., —NMe-, —N(C(═O)(C₁-C₆)alkyl))-, e.g., —NAc—, —NAr^{E3}- and —NC(═O)Ar^{E3}-. In some embodiments, Q^{E3} is —NAr^{E3}. In some embodiments, Q^{E3} is —NC(═O)Ar^{E3}-. In some embodiments, the —(C₁-C₃)alkylene-Q^{E3}-(C₁-C₃)alkylene- group is a group of the formula —(CH₂)₁₋₃-Q^{E3}-(CH₂)₁₋₃—, e.g., —(CH₂)₂-Q^{E3}-(CH₂)₂—. The group may be a pyrrolidine, piperidine or piperazine, morpholine or thiomorpholine ring. Ar^{E3} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{E3}, —C(═O)OR^{E}, —C(═O)NR^{E3}₂, —C(═NR^{E3})NR^{E3}₂, —OR^{E3}, —OC(═O)(C₁-C₆)alkyl, —OC(═O)O(C₁-C₆)alkyl, —OC(═O)NR^{E3}₂, —NR^{E3}₂, —NR^{E3}C(═O)R^{E3}, —NR^{E3}C(═O)O(C₁-C₆)alkyl, —NR^{E3}C(═O)NR^{E3}₂, —NR^{E3}SO₂R^{E3}, —SR^{E3}, —S(O)R^{E3}, —SO₂R^{E3}, —OSO₂(C₁-C₆)alkyl, —SO₂NR^{E3}₂, (C₁-C₈)perfluoroalkyl, —(C₂-C₆)alkylene-OR^{E3}, —O(C₂-C₆)alkylene-N((C₁-C₆)alkyl)₂, —P(═O)(OR^{E3})₂ and —OP(═O)(OR^{E3})₂; wherein each R^{E3} is independently H or (C₁-C₆)alkyl. Ar^{E3} may be a substituted or unsubstituted monocyclic aromatic ring, e.g., substituted or unsubstituted phenyl, or, e.g., (C₂-C₅)heteroaryl, e.g., pyridyl, pyrazinyl, pyrimidinyl, or pyridazinyl.

In some embodiments of the compounds of formula (I-A), D⁷ is C-E⁷. In some such embodiments, E⁷ may be, e.g., H, (C₁-C₆)alkyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —OR^{E7}, —Ar^{E7}, —OAr^{E7}, —O((C₁-C₆)alkylene)Ar^{E7}, —OC(═O)(C₁-C₆)alkyl, —NR^{E7}₂, —NR^{E7}Ar^{E7}, —NR^{E7}(C₁-C₆)alkylene)Ar^{E7}, —NR^{E7}C(═O)R^{E7}, or —NR^{E7}C(═O)Ar^{E7}. In some such embodiments, E⁷ may be, e.g., H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph, In some such embodiments, E⁷ may be, e.g., H or —NO₂.

In some embodiments of the compounds of formula (I-A), D⁸ is C-E⁸. In some such embodiments, E⁸ may be, e.g., H, (C₁-C₆)alkyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —OR^{E8}, —Ar^{E8}, —OAr^{E8}, —O((C₁-C₆)alkylene)Ar^{E8}, —OC(═O)(C₁-C₆)alkyl, —NR^{E8}₂, —NR^{E8}Ar^{E8}, NR^{E8}((C₁-C₆)alkylene)Ar^{E8}, —NR^{E8}C(═O)R, or —NR^{E8}C(═O)Ar^{E8}. In some such embodiments, E⁸ may be, e.g., H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph,

The compounds of formula (I) can include compounds of formula (I-B) or (I-C):

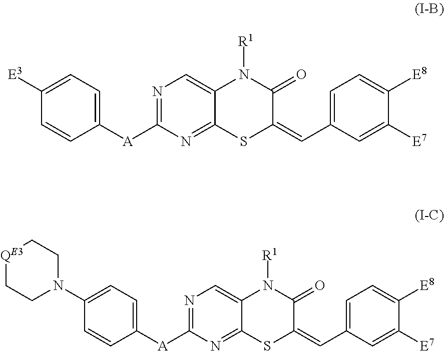

or a salt thereof, wherein:

A is selected from O, NR⁴ and S(O)ₘ;

R¹ is H or (C₁-C₆)alkyl;

E³ is selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(═O)OR⁷, —C(═O)NR⁷₂, —C(—NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₆)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂; and a substituted or unsubstituted heterocyclic ring linked via a nitrogen atom, e.g., a 5- to 7-membered ring, e.g., a pyrrolidine, piperidine, piperazine, morpholine or thiomorpholine ring; and

each R⁷ is independently selected from H and (C₁-C₆)alkyl;

Q^{E3} is selected from a bond, —CH₂—, —CH((C₁-C₆)alkyl)-,

e.g., —CHMe-, —C((C₁-C₆)alkyl)₂-,
e.g., —CMe₂-, —CHAr^{E3}, —C((C₁-C₆)alkyl)Ar^{E3}, —O—, —S—, —NH—, —N((C₁-C₈)alkyl)-,
e.g., —NMe-, —N(C(═O)(C₁-C₆)alkyl))-, e.g., —NAc—, —NAr^{E3}- and —NC(═O)Ar^{E3}-;

Ar^{E3} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(O)R^{E3}, —C(═O)OR^{E3}, —C(═O)NR^{E3}₂, —C(═NR^{E3})NR^{E3}₂, —OR^{E3}, —OC(═O)O(C₁-C₆)alkyl, —OC(═O)O(C₁-C₆)alkyl, —OC(═O)NR^{E3}, —NR^{E3}, —NR^{E3}C(═O)R^{E3}, —NR C(═O)O(C₁-C₆)alkyl, —NR^{R3}C(═O)NR₂, —NR^{E3}SO₂R^{E3}, —SR^{E3}, —S(O)R^{E3}, —SO₂R^{E3}, —OSO₂(C₁-C₆)alkyl, —SO₂NR^{E3}₂, (C₁-C₈)perfluoroalkyl, —(C₂-C₆)alkylene-OR^{E3}, —O(C₂-C₆)alkylene-N((C₁-C₆)alkyl)₂, —P(═O)(OR^{E3})₂ and —OP(═O)(OR^{E3})₂; wherein each R^{E3} is independently H or (C₁-C₆)alkyl. Ar^{E3} may be a substituted or unsubstituted monocyclic aromatic ring, e.g., substituted or unsubstituted phenyl, or, e.g., (C₂-C₅)heteroaryl, e.g., pyridyl, pyrazinyl, pyrimidinyl, or pyridazinyl;

E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar, —NR^{E7}((C₁-C₆)alkylene)Ar^{E7}, —NR^{E7}C(═O)R^{E7}, —NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N((C₁-C₈)alkyl)₂, —P(═O)(OR^{E7})₂ and —OP(═O)(OR^{E7})₂; wherein each R^{E7} is independently selected from H and (C₁-C₆)alkyl; and wherein each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E7}, —C(═O)OR, —C(═O)NR^{E7}₂, —C(—NR^{E7})NR^{E7}₂, —OR^{E7}, —OC(═O)O(C₁-C₆)alkyl, —OC(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl and —(C₂-C₆)alkylene-OR^{E7}; and

E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —C(═N^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(—O)O(C₁-C₆)alkyl, —OC(═O)O(C₁-C₆)alkyl, —OC(═O)NR^{E8}, —NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl, —(C₂-C₆)alkylene-OR^{E8}, —O(C₂-C₆)alkylene-N((C₁-C₆)alkyl)₂, —P(═O)(OR^{E8})₂ and —OP(═O)(OR^{E8})₂; wherein each R^{E8} is independently selected from H and (C₁-C₆)alkyl; and wherein each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E8}, —C(═O)OR^{E8}, —C(O)NR^{E8}₂, —C(═NR^{E8})NR₂, —OR^{E8}, —OC(═O)(C₁-C₆)alkyl, —OC(═O)O(C₁-C₆)alkyl, —OC(═O)NR^{E8}, —NR^{E8}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8}.

Each of Ar^{E7} and Ar^{E8} may be, e.g., substituted or unsubstituted phenyl.

In some embodiments of the compounds of formula (I-B), E³ is a pyrrolidine, piperidine, piperazine or morpholine ring substituted with one or more alkyl groups, e.g., 1, 2, 3, 4, or 5 alkyl groups. When E³ is a piperazine ring, the second nitrogen of the piperazine ring may be substituted with —C(═O)O(C₁-C₆)alkyl, e.g., an acetyl group.

In some embodiments of the compounds of formula (I-B), In some embodiments, E³ is a group according to the formula —NR⁸R⁹ wherein R⁸ and R⁹ in combination form a group according to the formula —(C₁-C₃)alkylene-Q^{E3}-(C₁-C₃)alkylene-. The ring formed by R⁸ and R⁹ and the nitrogen to which they are both attached may be a 5 to 7 membered ring. Q^{E3} is selected from a bond, —CH₂—, —CH((C₁-C₆)alkyl)-, e.g., —CHMe-, —C((C₁-C₆)alkyl)₂-, e.g., —CMe₂-, —CHAr^{E3}-, —C((C₁-C₆)alkyl)Ar^{E3}-, —O—, —S—, —NH—, —N((C₁-C₆)alkyl)-, e.g., —NMe-, —N(C(═O)(C₁-C₆)alkyl))-, e.g., —NAc—, —NAr^{E3}- and —NC(═O)Ar^{E3}-. In some embodiments, Q^{E3} is —NAr^{E3}-. In some embodiments, Q^{E3} is —NC(═O)Ar^{E3}-. In some embodiments, the —(C₁-C₃)alkylene-Q^{E3}-(C₁-C₃)alkylene- group is a group of the formula —(CH₂)₁₋₃-Q^{E3}-(CH₂)₁₋₃—, e.g., —(CH₂)₂-Q^{E3}-(CH₂)₂—. The group may be a pyrrolidine, piperidine or piperazine, morpholine or thiomorpholine ring. Ar^{E3} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{E3}, —C(═O)OR^{E3}, —C(═O)NR^{E3}₂, —C(═NR^{E3})NR^{E3}₂, —OR^{E3}, —OC(═O)(C₁-C₆)alkyl, —OC(═O)O(C₁-C₆)alkyl, —OC(═O)NR^{E3}₂, —NR^{E3}₂, —NR^{E3}C(═O)R^{E3}, —NR^{E3}C(═O)O(C₁-C₆)alkyl, —NR^{E3}C(═O)NR^{E3}₂, —NR^{E3}SO₂R^{E3}, —SR^{E3}, —S(O)R^{E3}, —SO₂R^{E3}, —OSO₂(C₁-C₆)alkyl, —SO₂NR^{E3}₂, (C₁-C₈)perfluoroalkyl, —(C₂-C₆)alkylene-OR^{E3}, —O(C₂-C₆)alkylene-N((C₁-C₆)alkyl)₂, —P(═O)(OR^{E3})₂ and —OP(═O)(OR^{E3})₂; wherein each R^{E3} is independently H or (C₁-C₆)alkyl. Ar^{E3} may be a substituted or unsubstituted monocyclic aromatic ring, e.g., substituted or unsubstituted phenyl, or, e.g., (C₂-C₅)heteroaryl, e.g., pyridyl, pyrazinyl, pyrimidinyl, or pyridazinyl.

In some embodiments of the compounds of formula (I-C), Q^{E3} is a bond. In some embodiments, Q^{E3} is —CH₂—, —CH((C₁-C₆)alkyl)-, e.g., —CHMe-, —C((C₁-C₆)alkyl)₂-, e.g., —CMe₂-, —CHAr^{E3}-, or —C((C₁-C₆)alkyl)Ar^{E3}. In some embodiments, Q^{E3} is —O—. In some embodiments, Q^{E3} is —S—. In some embodiment, Q^{E3} is —NH—, —N((C₁-C₆)alkyl)-, e.g., —NMe-, —N(C(═O)(C₁-C₆)alkyl))-, e.g., —NAc—, —NAr^{E3}- or —NC(═O)Ar^{E3}.

In some embodiments of the compounds of formulae (I-B) and (I-C), A is NR⁴, e.g., NH. In some embodiments, A is O.

The compounds of formula (I) can include compounds of formula (I-D):

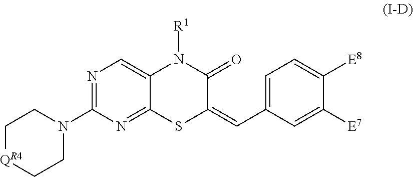

or a salt thereof, wherein:

R¹ is H or (C₁-C₆)alkyl;

Q^{R4} is selected from a bond, —CH₂—, —CH((C₁-C₆)alkyl)-, e.g., —CHMe-, —C((C₁-C₆)alkyl)₂-, e.g., —CMe₂-, —CHAr^{R4}—, e.g., —CHPh-, —C((C₁-C₆)alkyl)Ar^{R4}-, e.g., —CMePh-, —O—, —S—, —NH—, —N((C₁-C₆)alkyl)-, e.g., —NMe-, —NC(═O)((C₁-C₆)alkyl)-, e.g., —NAc—, —NAr^{R4}—, e.g., —NPh- and —NC(═O)Ar^{R4}—, e.g., —NC(═O)Ph-;

Ar^{R4} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, C(═O)R^{R4}, —C(═O)OR^{R4}, —C(═O)NR^{R4}₂, —C(═NR^{R4})NR^{R4}₂, —OR^{R4}, —OC(═O)(C₁-C₆)alkyl, —OC(═O)O(C₁-C₆)alkyl, —OC(═O)NR^{R4}₂, —N^{R4}₂, —NR^{R4}C(═O)R^{R4}, —NR^{R4}C(═O)O(C₁-C₆)alkyl, —NR⁴C(═O)NR^{R4}, —NR^{R4}SO₂R^{R4}, —SR^{R4}, —S(O)R^{R4}, —SO₂R^{R4}, —OSO₂(C₁-C₆)alkyl, —SO₂NR^{R4}₂, (C₁-C₈)perfluoroalkyl, —(C₂-C₆)alkylene-OR^{R4}, —O(C₂-C₆)alkylene-N((C₁-C₆)alkyl)₂, —P(═O)(OR^{R4})₂ and —OP(═O)(OR^{R4})₂;

each R^{R4} is independently selected from H and (C₁-C₆)alkyl.

E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R^{E7}, NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —SO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N((C₁-C₆)alkyl)₂, —P(═O)(OR^{E7})₂ and —OP(═O)(OR^{E7})₂; wherein each R^{E7} is independently selected from H and (C₁-C₆)alkyl; and wherein each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E7}—C(═O)OR^{E7}, —C(═O)NR^{E7}₂, —C(═NR^{E7})NR^{E7}₂, —OR^{E7}, —OC(═O)(C₁-C₆)alkyl, —OC(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl and —(C₂-C₆)alkylene-OR^{E7}; and

E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, —NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl, —(C₂-C₆)alkylene-OR^{E8}, —O(C₂-C₆)alkylene-N((C₁-C₆)alkyl)₂, —P(═O)(OR^{E8})₂ and —OP(═O)(OR^{E8})₂; wherein each R^{E8} is independently selected from H and (C₁-C₆)alkyl; and wherein each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E8}, —C(═O)OR^{E8}, —C(═O)NR^{E8}₂, —C(═NR^{E8})NR^{E8}₂, —OR^{E8}, —OC(═O)(C₁-C₆)alkyl, —OC(═O)O(C₁-C₆)alkyl, —OC(═O)NR^{E8}₂, —NR^{E8}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8}.

Each of Ar^{E7} and Ar^{E8} may be, e.g., substituted or unsubstituted phenyl.

In some embodiments of the compounds of formula (I-D), Q^{R4} is —NAr^{R4}-.

In some embodiments of the compounds of formula (I-D), Q^{R4} is —CHAr^{R4}-.

In some embodiments of the compounds of formula (I-D), Ar^{R4} may be a substituted or unsubstituted monocyclic aromatic ring, e.g., substituted or unsubstituted phenyl, or, e.g., (C₂-C₅)heteroaryl, e.g., a pyridyl, pyrazinyl, pyrimidinyl, or pyridazinyl ring.

In some embodiments of the compounds of formulae (I-A), (I-B), (I-C) and (I-D), R¹ is H.

In some embodiments of the compounds of formulae (I-A), (I-B), (I-C) and (I-D), E⁷ may be, e.g., H, (C₁-C₆)alkyl, halogen, (C₁-C₆)haloalkyl, —CN, —NO₂, —C(═O)R^{E7}, —OC(O)Ar^{E7}, —C(═O)OR^{E7}, —C(═O)NR^{E7}, —OR^{E7}, —Ar^{E7}, —OAr^{E7}, —O((C₁-C₆)alkylene)Ar^{E7}, —OC(═O)(C₁-C₆)alkyl, —NR^{E7}₂, —NR^{E7}Ar^{E7}, —NR^{E7}((C₁-C₆)alkylene)Ar^{E7}, —NR^{E7}C(═O)R^{E7}, or —NR^{E7}C(═O)Ar^{E7}. In some such embodiments, E⁷ may be, e.g., H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph, In some such embodiments, E⁷ may be, e.g., H or —NO₂.

In some embodiments of the compounds of formulae (I-A), (I-B), (I-C) and (I-D), E⁸ may be, e.g., H, (C₁-C₆)alkyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —OR^{E8}, —Ar^{E8}, —OAr^{E8}, —O((C₁-C₆)alkylene)Ar^{E8}, —OC(═O)(C₁-C₆)alkyl, —NR^{E8}₂, —NR^{E8}Ar^{E8}, —NR^{E8}((C₁-C₆)alkylene)Ar^{E8}, —NR^{E8}C(═O)R^{E8}, or —NR^{E8}C(═O)Ar^{E8}. In some such embodiments, E⁸ may be, e.g., H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph,

Compounds of formula (I) include, e.g., compounds of the following formulae and salts, e.g., pharmaceutically acceptable salts, thereof:
(E)-7-(4-fluorobenzylidene)-2-(4-(4-methylpiperazin-1-yl)phenylamino)-5H-pyrimido[4,5-b][1,4]thiazin-6(7H)-one (1a);
(E)-2-(4-(4-methylpiperazin-1-yl)phenylamino)-7-(4-nitrobenzylidene)-5H-pyrimido[4,5-b][1,4]thiazin-6(7H)-one (1b);
(E)-4-((2-(4-(4-methylpiperazin-1-yl)phenylamino)-6-oxo-5H-pyrimido[4,5-b][1,4]thiazin-7(6H)-ylidene)methyl)phenyl acetate (1c);
(E)-N-(7-(4-methoxy-3-nitrobenzylidene)-6-oxo-6,7-dihydro-5H-pyrimido[4,5-b][1,4]thiazin-2-yl)-N-(4-(4-methylpiperazin-1-yl)phenyl)acetamide (1d);
(E)-7-(4-nitrobenzylidene)-2-(4-(pyridin-2-yl)piperazin-1-yl)-5H-pyrimido[4,5-b][1,4]thiazin-6(7H)-one (1e);
(E)-2-(4-(4-acetylpiperazin-1-yl)phenoxy)-7-(4-nitrobenzylidene)-5H-pyrimido[4,5-b][1,4]thiazin-6(7B)-one (1f);
(E)-N-(7-((1-acetyl-1H-indol-3-yl)methylene)-6-oxo-6,7-dihydro-5H-pyrimido[4,5-b][1,4]thiazin-2-yl)-N-(4-(4-methylpiperazin-1-yl)phenyl)acetamide (1g);
(E)-7-(4-aminobenzlidene)-2-(4-(4-methylpiperazin-1-yl)phenylamino)-5H-pyrimido[4,5-b][1,4]thiazin-6(7B)-one (1h);
(E)-4-((6-oxo-2-(4-(pyridin-2-yl)piperazin-1-yl)-5H-pyrimido[4,5-b][1,4]thiazin-7(6H)-ylidene)methyl)phenyl acetate (1i);
(E)-4-((6-oxo-2-(4-(pyridin-2-yl)piperidin-1-yl)-5H-pyrimido[4,5-b][1,4]thiazin-7(6H)-ylidene)methyl)phenyl acetate;
(E)-7-(4-nitrobenzylidene)-2-(4-(pyrimidin-2-yl)piperazin-1-yl)-5H-pyrimido[4,5-b][1,4]thiazin-6(7H)-one (1j);
(E)-2-((4-morpholinophenyl)amino)-7-(4-nitrobenzylidene)-5H-pyrimido[4,5-b][1,4]thiazin-6(7H)-one (1k);
(E)-4-((2-(N-(4-(4-methylpiperazin-1-yl)phenyl)acetamido)-6-oxo-5H-pyrimido[4,5-b][1,4]thiazin-7(6H)-ylidene)methyl)benzoic acid (1l);
(E)-2-((4-(4-methylpiperidin-1-yl)phenyl)amino)-7-(4-nitrobenzylidene)-5H-pyrimido[4,5-b][1,4]thiazin-6(7H)-one (1m);
(E)-4-((2-((4-(4-methylpiperazin-1-yl)phenyl)amino)-6-oxo-5H-pyrimido[4,5-b][1,4]thiazin-7(6H)-ylidene)methyl)benzonitrile (1n);
(E)-7-(4-(benzyloxy)benzylidene)-2-((4-(4-methylpiperazin-1-yl)phenyl)amino)-5H-pyrimido[4,5-b][1,4]thiazin-6(7HT)-one (1o);
(E)-4-((2-((4-(4-methylpiperazin-1-yl)phenyl)amino)-6-oxo-5H-pyrimido[4,5-b][1,4]thiazin-7(6H)-ylidene)methyl)benzamide (1p); and
(E)-7-(4-hydroxybenzylidene)-2-((4-(4-methylpiperazin-1-yl)phenyl)amino)-5H-pyrimido[4,5-b][1,4]thiazin-6(7B)-one (1q).

This disclosure provides compounds of formula (I-E) or a pharmaceutically acceptable salt thereof, wherein:

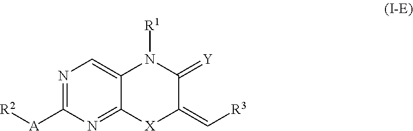

A is selected from O, NR⁴ and S(O)ₘ;

R¹ is hydrogen or a substituted or unsubstituted (C₁-C₁₀)hydrocarbyl;

R² is substituted or unsubstituted (C₁-C₁₀)hydrocarbyl;

R³ is selected from substituted or unsubstituted (C₆-C₁₀)aryl and substituted or unsubstituted (C₂-C₉)heteroaryl;

R¹ is selected from hydrogen, (C₁-C₆)alkyl and —C(═O)R⁵, wherein when R⁴ and R² are bound to the same nitrogen atom, R⁴ and R² can come together to form a substituted or unsubstituted heterocyclyl;

R⁵ is selected from hydrogen and (C₁-C₆)alkyl;

R⁶ is selected from hydrogen, —OH, (C₁-C₆)alkyl and —O—(C₁-C₆)alkyl;

X is S(O)ₙ;

Y is selected from O, S and NR⁶;

m is an integer selected from 0, 1 and 2; and

is n is an integer selected from 0, 1 and 2.

In some embodiments, A is NR⁴.

In some embodiments, R¹ is selected from hydrogen, (C₁-C₆)alkyl, (C₂-C₆)alkenyl, (C₂-C₆)alkynyl and (C₃-C₇)cycloalkyl. For example, R¹ can be H. In some embodiments, R¹ is substituted. For example, R¹ can be substituted with one or more of halogen, —OR′, —(CH₂)_{q}OR′, —SR′, —NO₂, —NR′R″, —CN, (C₁-C₆)hydrocarbyl, (C₁-C₆)haloalkyl, —C(═O)R′, —C(═O)OR′, —C(═O)NR′R″, —C(═NR′)NR′₂, —OC(═O)R′, —OC(═O)OR′, —OC(═O)NR′₂, —O—(CH₂)_{q}OR′, —O—(CH₂)_{q}NR′R″, —O—(CH₂)_{q}-halo, —NR′C(═O)R′, —NR′C(═O)OR′, —NR′C(═O)NR′₂, —NR′SO₂R′, —S(O)R′, —SO₂R′, —O—SO₃R′, —O—SO₂R′, —SO₂NR′₂, —O—P(═O)(OR′)₂, —P(═O)(OR′)₂, 4-methylpiperazin-1-yl, 4-BOC-piperazin-1-yl, 4-acetylpiperazin-1-yl, —O-glycoside and —O-glucuronide; wherein q is an integer selected from 2, 3 and 4, and R′ and R″ are independently selected from H and (C₁-C₆)hydrocarbyl, or R′ and R″ in NR′R″ and —C(═O)NR′R″ come together to form a carbocyclic or heterocyclic ring, where the heteroatom is selected from O, S, NR′″, wherein R′″ is H or a (C₁-C₆)hydrocarbyl.

In some embodiments, R¹ is substituted with one or more of (C₁-C₆)alkyl, (C₂-C₆)alkenyl, (C₂-C₆)alkynyl, halogen, —CN, —NO₂, —C(═O)R′, —C(═O)OR′, —C(═O)NR′₂, —C(—NR′)NR′₂, —OR′, —OC(═O)O(C₁-C₆)alkyl, —OC(═O)O(C₁-C₆)alkyl, —OC(═O)NR′₂, —NR′₂, —NR′C(═O)R′, —NR′C(═O)O(C₁-C₆)alkyl, —NR′C(═O)NR′₂, —NR′SO₂R′, —SR′, —S(O)R′, —SO₂R′, —OSO₂(C₁-C₆)alkyl, —SO₂NR′₂, (C₂-C₈)heterocyclyl, (C₁-C₆)perfluoroalkyl (e.g., —CF₃), (C₂-C₆)alkylene-OR′, —O(C₂-C₆)alkylene-N((C₁-C₆)alkyl)₂, —P(═O)(OR″), —OP(═O)(OR″)₂, 4-methylpiperazin-1-yl, 4-BOC-piperazin-1-yl and 4-acetylpiperazin-1-yl, wherein R′ and R″ are independently selected from H and (C₁-C₆)alkyl.

R² can be selected from (C₁-C₆)alkyl, (C₃-C₇)cycloalkyl, substituted or unsubstituted —(CH₂)ᵣ₂(C₆-C₁₀)aryl, substituted or unsubstituted —(CH₂)ᵣ₂(C₂-C₇)heterocyclyl, substituted or unsubstituted (C₆-C₁₀)aryl and substituted or unsubstituted (C₂-C₉)heterocyclyl. In some embodiments, R² is a substituted (C₆-C₁₀)aryl. For example, R² can be a substituted C₆ is aryl, such as a para-substituted C₆ aryl.

In some embodiments, R² is substituted. For example, R² can be substituted with one or more of halogen, —OR′, (CH₂)_{q}OR′, —SR′, —NO₂, —NR′R″, —CN, (C₁-C₆)hydrocarbyl, (C₁-C₆)haloalkyl, —C(═O)R′, —C(═O)OR′, —C(═O)NR′R″, —C(═NR′)N₂, —OC(═O)R′, —OC(O)OR′, —OC(═O)NR′₂, —(CH₂)_{q}OR′, —O—(CH₂)_{q}NR′R″, —O—(CH₂)_{q}-halo, —NR′C(═O)R′, —NR′C(═O)OR′, NR′C(═O)NR′₂, —NR′SO₂R′, —S(O)R′, —SO₂R′, —O—SO₃R′, —O—SO₂R′, —SO₂NR′₂, —O—P(═O)(OR₂, —P(═O)(OR)₂, 4-methylpiperazin-1-yl, 4-BOC-piperazin-1-yl, 4-acetylpiperazin-1-yl, —O-glycoside and —O-glucuronide; wherein q is an integer selected from 2, 3 and 4, and R′ and R″ are independently selected from H and (C₁-C₆)hydrocarbyl, or R′ and R″ in NR′R″ and —C(═O)NR′R″ come together to form a carbocyclic or heterocyclic ring, where the heteroatom is selected from O, S, NR′″, wherein R′″ is H or a (C₁-C₆)hydrocarbyl.

In some embodiments, R² is substituted with one or more of (C₁-C₆)alkyl, (C₂-C₆)alkenyl, (C₂-C₆)alkynyl, halogen, —CN, —NO₂, —C(═O)R′, —C(═O)OR′, —C(═O)NR′₂, —C(═NR)NR′₂, —OR′, —OC(═O)(C₁-C₆)alkyl, —OC(═O)O(C₁-C₆)alkyl, —OC(═O)NR′₂, —NR′₂, —NRC(═O)R′, —NRC(═O)O(C₁-C₆)alkyl, —NR′C(═O)NR′₂, —NR′SO₂R′, —SR′, —S(O)R′, —SO₂R′, —OSO₂(C₁-C₆)alkyl, —SO₂NR′₂, (C₂-C₉)heterocyclyl, (C₁-C₆)perfluoroalkyl (e.g., —CF₃), (C₂-C₆)alkylene-OR′, —O(C₂-C₆)alkylene-N((C₁-C₆)alkyl), —P(═O)(OR″)₂, —OP(═O)(OR″)₂, 4-methylpiperazin-1-yl, 4-BOC-piperazin-1-yl and 4-acetylpiperazin-1-yl, wherein R′ and R″ are independently selected from H and (C₁-C₆)alkyl.

In some embodiments, R⁴ is H or —C(═O)R⁵. For example, R⁵ can be CH₃. In some embodiments, R⁴ and R² come together to form a substituted heterocyclyl. For example, the substituted heterocyclyl can be a substituted piperidinyl.

In some embodiments, Y is O. In some embodiments, n is 0. R³ can be a substituted (C₆-C₁₀)aryl. For example, R³ can be a substituted C₆ aryl, such as a para-substituted C₆ aryl.

A compound of formula (I-E) can include, e.g., a compound of formula (I-F):

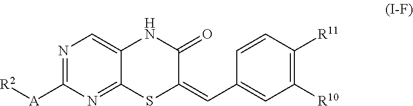

or a pharmaceutically acceptable salt thereof, wherein:

A is selected from O, NR⁴ and S(O)ₘ;

R² is substituted or unsubstituted (C₁-C₁₀)hydrocarbyl;

R⁴ is selected from hydrogen, (C₁-C₆)alkyl and —C(═O)R⁵, wherein when R⁴ and R² are bound to the same nitrogen atom, R⁴ and R² can come together to form a substituted or unsubstituted heterocyclyl;

R⁵ is selected from hydrogen and (C₁-C₆)alkyl;

R¹⁰ and R¹¹ are independently selected from hydrogen, (C₁-C₆)alkyl, (C₂-C₆)alkenyl, (C₂-C₆)alkynyl, halogen, —CN, —NO₂, —C(═O)R¹², —C(═O)OR¹², —C(═O)NR¹²₂, —C(═NR¹²)NR¹²₂, —OR¹², —OC(═O)(C₁-C₆)alkyl, —OC(═O)O(C₁-C₆)alkyl, —OC(═O)NR¹²₂, —NR¹²₂, —NR¹²C(═O)R¹², —NR¹²C(═O)O(C₁-C₆)alkyl, —NR¹²C(═O)NR¹²₂, —NR¹²SO₂R¹², —SR¹², —S(O)R¹², —SO₂R¹², —OSO₂(C₁-C₆)alkyl, —SO₂NR¹²₂,

(C₁-C₈)perfluoroalkyl, —(C₂-C₆)alkylene-OR¹², —O(C₂-C₆)alkylene-N((C₁-C₆)alkyl)₂, —P(═O)(OR¹³) and —OP(═O)(OR¹³)₂;

each R¹² and R¹³ is independently selected from hydrogen and (C₁-C₆)alkyl;

m is an integer selected from 0, 1 and 2; and

r is an integer selected from 1, 2, 3 and 4.

In some embodiments, A is NR⁴.

R² can be selected from (C₁-C₆)alkyl, (C₃-C₇)cycloalkyl, substituted or unsubstituted —(CH₂)ᵣ(C₆-C₁₀)aryl, substituted or unsubstituted —(CH₂)ᵣ (C₂-C₇)heterocyclyl, substituted or unsubstituted (C₆-C₁₀)aryl, and substituted or unsubstituted (C₂-C₉)heterocyclyl. In some embodiments, R² is a substituted (C₆-C₁₀)aryl. For example, R² can be a substituted C₆ aryl, such as a para-substituted C₆ aryl.

In some embodiments, R² is substituted. For example, R² can be substituted with one or more of halogen, —OR′, (CH₂)_{q}OR′, —SR′, —NO₂, —NR′R″, —CN, (C₁-C₆)hydrocarbyl, (C₁-C₆)haloalkyl, —C(═O)R′, —C(═O)OR′, —C(═O)NR′R″, —C(═NR′)NR′₂, —OC(═O)R′, —OC(═O)OR′, —OC(═O)NR′₂, —O—(CH₂)_{q}OR′, —O—(CH₂)_{q}NR′R″, —O—(CH₂)_{q}-halo, —NR′C(═O)R′, —NR′C(═O)OR′, —NR′C(═O)NR′₂, —NR′SO₂R′, —S(O)R′, —SO₂R′, —O—SO₃R′, —O—SO₂R′, —SO₂NR′₂, —O—P(═O)(OR′)₂, —P(═O)(OR′)₂, 4-methylpiperazin-1-yl, 4-BOC-piperazin-1-yl, 4-acetylpiperazin-1-yl, —O-glycoside and —O-glucuronide; wherein q is an integer selected from 2, 3 and 4, and R′ and R″ are independently selected from H and (C₁-C₆)hydrocarbyl, or R′ and R″ in NR′R″ and —C(═O)NR′R″ come together to form a carbocyclic or heterocyclic ring, where the heteroatom is selected from O, S, NR′″, wherein R′″ is H or a (C₁-C₆)hydrocarbyl.

In some embodiments, R² is substituted with one or more of (C₁-C₆)alkyl, (C₂-C₆)alkenyl, (C₂-C₆)alkynyl, halogen, —CN, —NO₂, —C(═O)R′, —C(═O)OR′, —C(═O)NR′₂, —C(═NR′)NR′₂, —OR′, —OC(═O)(C₁-C₆)alkyl, —OC(═O)O(C₁-C₆)alkyl, —OC(═O)NR′₂, —NR′₂, —NR′C(═O)R′, —NR′C(═O)O(C₁-C₆)alkyl, —NR′C(═O)NR′₂, —NR′SO₂R′, —SR′, —S(O)R′, —SO₂R′, —OSO₂(C₁-C₆)alkyl, —SO₂NR′₂, (C₂-C₉)heterocyclyl, (C₁-C₆)perfluoroalkyl (e.g., —CF₃), (C₂-C₆)alkylene-OR′, —O(C₂-C₆)alkylene-N((C₁-C₆)alkyl)₂, —P(═O)(OR″)₂, —OP(═O)(OR″)₂, 4-methylpiperazin-1-yl, 4-BOC-piperazin-1-yl and 4-acetylpiperazin-1-yl, wherein R′ and R″ are independently selected from H and (C₁-C₆)alkyl.

In some embodiments, R⁴ is H or —C(═O)R⁵. For example, R⁵ can be CH₃. In some embodiments, R¹ and R² come together to form a substituted heterocyclyl. For example, the substituted heterocyclyl is a substituted piperidinyl.

In some embodiments, R¹¹ is H or —NO₂. In some embodiments, R¹⁰ is selected from halogen, —NH₂, —NO₂, —OR¹⁰ and —OC(═O)(C₁-C₆)alkyl. For example, R¹⁰ can be F or —NO₂.

A compound of formula (I-E) or formula (I-F) can include, e.g., a compound of formula (1F):

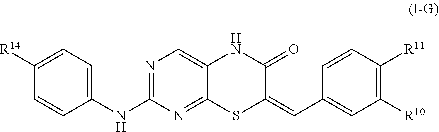

or a pharmaceutically acceptable salt thereof, wherein:

R¹⁰ and R¹¹ are independently selected from hydrogen, (C₁-C₆)alkyl, (C₂-C₆)alkenyl, (C₂-C₆)alkynyl, halogen, —CN, —NO₂, —C(═O)R¹², —C(O)OR¹², —C(═O)NR¹²₂, —C(═N¹²)NR¹²₂, —OR¹², —OC(═O)(C₁-C₆)alkyl, —OC(═O)O(C₁-C₆)alkyl, —OC(═O)NR¹²₂, —NR¹²₂, —NR¹²C(═O)R¹², —NR¹²C(═O)O(C₁-C₆)alkyl, —NR¹²C(═O)NR¹²₂, —NR¹²SO₂R¹², —SR¹², —S(O)R¹², —SO₂R¹², —OSO₂(C₁-C₆)alkyl, —SO₂NR¹²₂, (C₁-C₈)perfluoroalkyl, —(C₂-C₆)alkylene-OR¹², —O(C₂-C₆)alkylene-N((C₁-C₆)alkyl)₂, —P(═O)(OR¹³)₂ and —OP(═O)(OR¹³)₂;

each R¹² and R¹³ is independently selected from hydrogen and (C₁-C₆)alkyl; and

R¹⁴ is a substituted or unsubstituted (C₂-C₉)heterocyclyl.

In some embodiments, R¹⁴ is a substituted (C₂-C₉)heterocyclyl. For example, R¹⁴ can be a substituted piperazinyl.

### B. Compounds of Formula II:

Provided herein is a compound of formula (II):

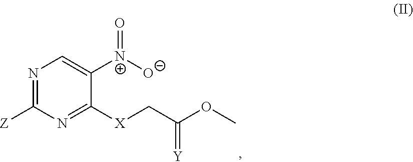

or a salt such as a pharmaceutically acceptable salt thereof.

In the compounds of Formula (II):

X and Y may be as defined herein for the compounds of formula (I); and

Z is a leaving group, e.g., a halogen.

In particular embodiments of the compounds of formula (II), X and Y may be as defined herein for particular embodiment of the compounds of formula (I), including, but not limited to, the compounds of formula (I-A), (I-B), (I-C), (I-D), (I-E), (I-F) and (I-G) and any of the embodiments thereof described herein.

In some of the compounds of formula (II), or a salt such as a pharmaceutically acceptable salt thereof:

X is S(O)ₙ;

Y is selected from O, S and NR⁶;

Z is a halogen;

R⁶ is selected from hydrogen, —OH, (C₁-C₆)alkyl and —O-(C₁-C₆)alkyl; and

n is an integer selected from 0, 1 and 2.

In some embodiments, X can be S. In some embodiments, Y is O. In some embodiments, Z is Cl.

Compounds of formula (II) can include, e.g., methyl 2-(2-chloro-5-nitropyrimidin-4-ylthio)acetate (2) and salts thereof, e.g. pharmaceutically acceptable salts thereof.

### C. Compounds of Formula (III).

Provided herein is a compound of formula (III):

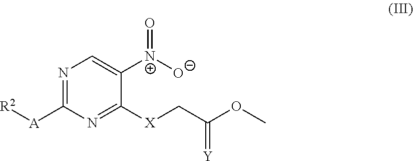

or a pharmaceutically acceptable salt thereof.

In the compounds of Formula (III), X, Y, A and R² may be as defined above for the compounds of formula (I).

In particular embodiments of the compounds of formula (III), X, Y, A and R² may be as defined herein for particular embodiment of the compounds of formula (I), including, but not limited to, the compounds of formula (I-A), (I-B), (I-C), (I-D), (I-E), (I-F) and (I-G) and any of the other embodiments thereof described herein.

In some embodiments of the compounds of formula (III):
A is selected from O, NR⁴ and S(O)ₘ;
R² is selected from substituted or unsubstituted (C₁-C₁₀)hydrocarbyl and substituted or unsubstituted heterocyclyl;
R⁴ is selected from H, (C₁-C₆)alkyl and —C(═O)R⁵;

or R² or R⁴ in combination with the nitrogen to which they are attached form a substituted or unsubstituted heterocyclyl;
R⁵ is selected from H and (C₁-C₆)alkyl;
X is S(O)ₙ;
Y is selected from O, S and NR⁶;
R⁶ is selected from H, —OH, (C₁-C₆)alkyl and —O—(C₁-C₆)alkyl;
m is an integer selected from 0, 1 and 2; and
n is an integer selected from 0, 1 and 2.

In some embodiments of the compounds of formula (III):
A is selected from O, NR⁴ and S(O)ₘ;
R² is substituted or unsubstituted (C₁-C₁₀)hydrocarbyl;
R⁴ is selected from hydrogen, (C₁-C₆)alkyl and —C(═O)R⁵, wherein when R⁴ and R² are bound to the same nitrogen atom, R⁴ and R² can come together to form a substituted or unsubstituted heterocyclyl;
R⁵ is selected from hydrogen and (C₁-C₆)alkyl;
R¹ is selected from hydrogen, —OH, (C₁-C₆)alkyl and —O—(C₁-C₆)alkyl;
X is S(O)ₙ;
Y is selected from O, S and NR⁶;
m is an integer selected from 0, 1 and 2;
n is an integer selected from 0, 1 and 2; and
r is an integer selected from 1, 2, 3 and 4.

In some embodiments, A is NR⁴. In some embodiments, Y is O. In some embodiments, X is S. In some embodiments, R⁴ is H or R⁴ and R² come together to form a substituted heterocyclyl such as a substituted piperazinyl.

In some embodiments, R² is selected from (C₁-C₆)alkyl, (C₂-C₆)alkenyl, (C₂-C₆)alkynyl, substituted or unsubstituted (C₃-C₇)cycloalkyl, substituted or unsubstituted (C₆-C₁₀) aryl, substituted or unsubstituted —(CH₂)ᵣ—(C₆-C₁₀)aryl, substituted or unsubstituted (C₂-C₉)heterocyclyl, and substituted or unsubstituted —(CH₂)ᵣ—(C₂-C₇)heterocyclyl. In some embodiments, R² is a substituted (C₆-C₁₀)aryl. For example, R² can be a substituted C₆ aryl such as a para-substituted C₆ aryl.

In some embodiments, R² is substituted. For example, R² can be substituted with one or more of halogen, —OR′, (CH₂)_{q}OR′, —SR′, —NO₂, —NR′R″, —CN, (C₁-C₆)hydrocarbyl, (C₁-C₆)haloalkyl, —C(═O)R′, —C(═O)OR′, —C(═O)NR′R″, —C(═NR)NR′₂, —OC(═O)R′, —OC(═O)OR′, —OC(═O)NR′₂, —O—(CH₂)_{q}OR′, —O—(CH₂)NR′R″, —O—(CH₂)_{q}-halo, —NR′C(═O)R′, —NR′C(═O)OR′, —NRC(═O)NR′₂, —NR′SO₂R′, —S(O)R′, —SO₂R′, —O—SO₃R′, —O—SO₂R′, —SO₂NR′₂, —O—P(═O)(OR′)₂, —P(═O)(OR′)₂, 4-methylpiperazin-1-yl, 4-BOC-piperazin-1-yl, 4-acetylpiperazin-1-yl, —O-glycoside and —O-glucuronide; wherein q is an integer selected from 2, 3 and 4, and R′ and R″ are independently selected from H and (C₁-C₆)hydrocarbyl, or R′ and R″ in NR′R″ and —C(═O)NR′R″ come together to form a carbocyclic or heterocyclic ring, where the heteroatom is selected from O, S, NR′″, wherein R′″ is H or a (C₁-C₆)hydrocarbyl.

In some embodiments, R² is substituted with one or more of (C₁-C₆)alkyl, (C₂-C₆)alkenyl, (C₂-C₆)alkynyl, halogen, —CN, —NO₂, —C(═O)R′, —C(═O)OR′, —C(═O)NR′₂, —C(═NR′)NR′₂, —OR′, —OC(═O)(C₁-C₆)alkyl, —OC(═O)O(C₁-C₆)alkyl, —OC(═O)NR′₂, —NR′₂, —NR′C(═O)R′, —NR′C(═O)O(C₁-C₆)alkyl, —NR′C(═O)NR′₂, —NR′SO₂R′, —SR′, —S(O)R′, —SO₂R′, —OSO₂(C₁-C₆)alkyl, —SO₂NR′₂, (C₂-C₉)heterocyclyl, (C₁-C₆)perfluoroalkyl (e.g., —CF₃), (C₂-C₆)alkylene-OR′, —O(C₂-C₆)alkylene-N((C₁-C₆)alkyl)₂, —P(═O)(OR″)₂, —OP(═O)(OR″)₂, 4-methylpiperazin-1-yl, 4-BOC-piperazin-1-yl and 4-acetylpiperazin-1-yl, wherein R′ and R″ are independently selected from H and (C₁-C₆)alkyl.

Compounds of formula (III) include, e.g., the following compounds, and salts thereof:
methyl 2-(2-(4-(4-methylpiperazin-1-yl)phenylamino)-5-nitropyrimidin-4-ylthio) acetate (3a);
methyl 2-(5-nitro-2-(4-(pyridin-2-yl)piperazin-1-yl)pyrimidin-4-ylthio)acetate (3b);
methyl 2-(5-nitro-2-(4-(piperazin-1-yl)phenoxy)pyrimidin-4-ylthio)acetate (3c);
methyl 2-(2-(4-chlorophenylthio)-5-nitropyrimidin-4-ylthio)acetate (3d);
methyl 2-(2-(2,6-dichlorobenzylthio)-5-nitropyrimidin-4-ylthio)acetate (3e);
methyl 2-((5-nitro-2-(4-(pyrimidin-2-yl)piperazin-1-yl)pyrimidin-4-yl)thio)acetate (3f);
methyl 2-((2-((4-morpholinophenyl)amino)-5-nitropyrimidin-4-yl)thio)acetate (3g); and
methyl 2-((2-((4-(4-methylpiperidin-1-yl)phenyl)amino)-5-nitropyrimidin-4-yl)thio)acetate (3h).

### D. Compounds of Formula (IV):

Further provided herein is a compound of formula (IV):

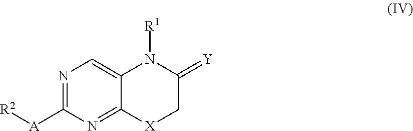

or a salt thereof.

In the compounds of Formula (IV), X, Y, A, R¹ and R² may be as defined above for the compounds of formula (I).

In particular embodiments of the compounds of formula (IV), X, Y, A, R¹ and R² may be as defined herein for particular embodiment of the compounds of formula (I), including, but not limited to, the compounds of formula (I-A), (I-B), (I-C), (I-D), (I-E), (I-F) and (I-G) and any of the other embodiments thereof described herein.

In some embodiments of the compounds of formula (IV):
A is selected from O, NR⁴ and S(O)ₘ;
R¹ is H or a substituted or unsubstituted (C₁-C₁₀)hydrocarbyl;
R² is selected from substituted or unsubstituted (C₁-C₁₀)hydrocarbyl and substituted or unsubstituted heterocyclyl;
R⁴ is selected from H, (C₁-C₆)alkyl and —C(═O)R⁵;
or R² or R⁴ in combination with the nitrogen to which they are attached form a substituted or unsubstituted heterocyclyl;
R⁵ is selected from H and (C₁-C₆)alkyl;
X is S(O)ₙ;
Y is selected from O, S and NR⁶;
R⁶ is selected from H, —OH, (C₁-C₆)alkyl and —O—(C₁-C₆)alkyl;
m is an integer selected from 0, 1 and 2; and
n is an integer selected from 0, 1 and 2.

In some embodiments of the compounds of formula (III):
A is selected from O, NR⁴ and S(O)ₘ;
R¹ is a hydrogen or substituted or unsubstituted (C₁-C₁₀)hydrocarbyl;
R² is substituted or unsubstituted (C₁-C₁₀)hydrocarbyl;
R⁴ is selected from hydrogen, (C₁-C₆)alkyl and —C(═O)R⁵, wherein when R⁴ and R² are bound to the same nitrogen atom, R⁴ and R² can come together to form a substituted or unsubstituted heterocyclyl;
R⁵ is selected from hydrogen and (C₁-C₆)alkyl;
R⁶ is selected from hydrogen, —OH, (C₁-C₆)alkyl and —O—(C₁-C₆)alkyl;
X is S(O)ₙ;
Y is selected from O, S and NR⁶;
m is an integer selected from 0, 1 and 2;
n is an integer selected from 0, 1 and 2; and
r is an integer selected from 1, 2, 3 and 4.

In some embodiments, A is NR⁴. In some embodiments, X is S. In some embodiments, Y is O.

In some embodiments, R¹ is selected from hydrogen, (C₁-C₆)alkyl, (C₂-C₆)alkenyl, (C₂-C₆)alkynyl and (C₃-C₇)cycloalkyl. For example, R¹ can be H. In some embodiments, R¹ is substituted. For example, R¹ can be substituted with one or more of halogen, —OR′, (CH₂)_{q}OR′, —SR′, —NO₂, —NR′R″, —CN, (C₁-C₆)hydrocarbyl, (C₁-C₆)haloalkyl, —C(═O)R′, —C(═O)OR′, —C(═O)NR′R″, —C(—NR′)NR′₂, —OC(═O)R′, —OC(═O)OR′, —OC(═O)NR′₂, —O—(CH₂)_{q}OR′, —O—(CH₂)NR′R″, —O—(CH₂)_{q}-halo, —NR′C(═O)R′, —NR′C(═O)OR′, —NR′C(═O)NR′₂, —NR′SO₂R′, —S(O)R′, —SO₂R′, —O—SO₃R′, —O—SO₂R′, —SO₂NR′₂, —O—P(═O)(OR′)₂, —P(═O)(OR′)₂, 4-methylpiperazin-1-yl, 4-BOC-piperazin-1-yl, 4-acetylpiperazin-1-yl, —O-glycoside and —O-glucuronide; wherein q is an integer selected from 2, 3 and 4, and R′ and R″ are independently selected from H and (C₁-C₆)hydrocarbyl, or R¹ and R″ in NR′R″ and —C(═O)NR′R″ come together to form a carbocyclic or heterocyclic ring, where the heteroatom is selected from O, S, NR′″, wherein R′″ is H or a (C₁-C₆)hydrocarbyl.

In some embodiments, R¹ is substituted with one or more of (C₁-C₆)alkyl, (C₂-C₆)alkenyl, (C₂-C₆)alkynyl, halogen, —CN, —NO₂, —C(═O)R′, —C(═O)OR′, —C(═O)NR′₂, —C(═NR′)NR′₂, —OR′, —OC(═O)(C₁-C₆)alkyl, —OC(═O)O(C₁-C₆)alkyl, —OC(═O)O(C₁-C₆)alkyl, —OC(═O)NR′₂, —NR′₂, —NR′C(═O)R′, —NR′C(═O)O(C₁-C₆)alkyl, —NR′C(═O)NR′₂, —NR′SO₂R′, —SR′, —S(O)R′, —SO₂R′, —OSO₂(C₁-C₆)alkyl, —SO₂NR′₂, (C₂-C₉)heterocyclyl, (C₁-C₆)perfluoroalkyl (e.g., —CF₃), (C₂-C₆)alkylene-OR′, —O(C₂-C₆)alkylene-N((C₁-C₆)alkyl)₂, —P(═O)(OR″)₂, —OP(═O)(OR″)₂, 4-methylpiperazin-1-yl, 4-BOC-piperazin-1-yl and 4-acetylpiperazin-1-yl, wherein R′ and R″ are independently selected from H and (C₁-C₆)alkyl.

R² can be selected from (C₁-C₆)alkyl, (C₃-C₇)cycloalkyl, substituted or unsubstituted —(CH₂)ᵣ(C₆-C₁₀)aryl, substituted or unsubstituted —(CH₂)ᵣ(C₂-C₇)heterocyclyl, substituted or unsubstituted (C₆-C₁₀)aryl and substituted or unsubstituted (C₂-C₉)heterocyclyl. In some embodiments, R² is a substituted (C₆-C₁₀)aryl. For example, R² can be a substituted C₆ aryl, such as a para-substituted C₆ aryl.

In some embodiments, R² is substituted. For example, R² can be substituted with one or more of halogen, —OR′, (CH₂)_{q}OR′, —SR′, —NO₂, —NR′R″, —CN, (C₁-C₆)hydrocarbyl, (C₁-C₆)haloalkyl, —C(═O)R′, —C(═O)OR′, —C(═O)NR′R″, —C(═NR)NR′₂, —OC(═O)R′, —OC(═O)OR′, —OC(═O)NR′₂, —O—(CH₂)_{q}OR′, —O—(CH₂)_{q}NR′R″, —O—(CH₂)_{q}-halo, —NR′C(═O)R′, —NR′C(═O)OR′, —NR′C(═O)NR′₂, —NR′SO₂R′, —S(O)R′, —SO₂R′, —O—SO₃R′, —O—SO₂R′, —SO₂NR′₂, —O—P(═O)(OR′)₂, —P(═O)(OR′)₂, 4-methylpiperazin-1-yl, 4-BOC-piperazin-1-yl, 4-acetylpiperazin-1-yl, —O-glycoside and —O-glucuronide; wherein q is an integer selected from 2, 3 and 4, and R′ and R″ are independently selected from H and (C₁-C₆)hydrocarbyl, or R′ and R″ in NR′R″ and —C(═O)NR′R″ come together to form a carbocyclic or heterocyclic ring, where the heteroatom is selected from O, S, NR′″, wherein R′″ is H or a (C₁-C₆)hydrocarbyl.

In some embodiments, R² is substituted with one or more of (C₁-C₆)alkyl, (C₂-C₆)alkenyl, (C₂-C₆)alkynyl, halogen, —CN, —NO₂, —C(═O)R′, —C(═O)OR′, —C(═O)NR′₂, —C(═NR′)NR′₂, —OR′, —OC(═O)(C₁-C₆)alkyl, —OC(═O)O(C₁-C₆)alkyl, —OC(═O)NR′₂, —NR′₂, —NR′C(═O)R′, —NR′C(═O)O(C₁-C₆)alkyl, —NR′C(═O)NR′₂, —NR′SO₂R′, —SR′, —S(O)R′, —SO₂R′, —OSO₂(C₁-C₆)alkyl, —SO₂NR′₂, (C₂-C₉)heterocyclyl, (C₁-C₆)perfluoroalkyl (e.g., —CF₃), (C₂-C₆)alkylene-OR′, —O(C₂-C₆)alkylene-N((C₁-C₆)alkyl)₂, —P(═O)(OR″)₂, —OP(O)(OR″)₂, 4-methylpiperazin-1-yl, 4-BOC-piperazin-1-yl and 4-acetylpiperazin-1-yl, wherein R′ and R″ are independently selected from H and (C₁-C₆)alkyl.

A compound of formula (IV) can include, e.g., the following compounds and salts thereof:
2-(4-(4-methylpiperazin-1-yl)phenylamino)-5H-pyrimido[4,5-b][1,4]thiazin-6(7H)-one (4a);
2-(4-(pyridin-2-yl)piperazin-1-yl)-5H-pyrimido[4,5-b][1,4]thiazin-6(7H)-one (4b);
2-(4-(piperazin-1-yl)phenoxy)-5H-pyrimido[4,5-b][1,4]thiazin-6(7H)-one (4c);
2-(2,6-dichlorobenzylthio)-5H-pyrimido[4,5-b][1,4]thiazin-6(7H)-one (4d);
2-(4-(pyrimidin-2-yl)piperazin-1-yl)-5H-pyrimido[4,5-b][1,4]thiazin-6(7H)-one (4e);
2-((4-Morpholinophenyl)amino)-5H-pyrimido[4,5-b][1,4]thiazin-6(7H)-one (4f); and
2-((4-(4-Methylpiperidin-1-yl)phenyl)amino)-5H-pyrimido[4,5-b][1,4]thiazin-6(7H)-one (4g).

### III. SYNTHESIS

Compounds provided herein, including salts thereof, can be prepared using known organic synthesis techniques and can be synthesized according to any of numerous possible synthetic routes.

The reactions for preparing compounds as described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of so appropriate protecting groups, can be readily determined by one skilled in the art. The chemistry of protecting groups can be found, e.g., in Protecting Group Chemistry, 1^{st} Ed., Oxford University Press, 2000; March's Advanced Organic chemistry: Reactions, Mechanisms, and Structure, 5^{th} Ed., Wiley-Interscience Publication, 2001; and Peturssion, S. et al., “Protecting Groups in Carbohydrate Chemistry,” J, Chem. Educ., 74(11), 1297 (1997) (each of which is incorporated herein by reference in their entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS) or thin layer chromatography (TLC). Compounds can be purified by those skilled in the art by a variety of methods, including high performance liquid chromatography (HPLC) (“Preparative LC-MS Purification: Improved Compound Specific Method Optimization” K. F. Blom, et al., J. Combi. Chem. 6(6) (2004), which is incorporated herein by reference in its entirety) and normal phase silica chromatography.

Compounds of formula (I), including, but not limited to compounds of formula (I), (I-A), (I-B), (I-C), (I-D), (I-E), (I-F) and (I-G) as described therein, and any other embodiments of the compounds according to claim 1 as described herein, can be prepared by reacting a compound of formula (IV):

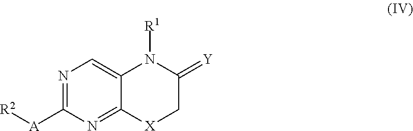

or a salt thereof, with a compound of formula (VI):

R³—C(═O)H (VI)

or a salt thereof, to form the compound of formula (I).

In particular, a compound of formula (I) may be prepared upon reaction of a compound of formula (IV) and formula (VI) in the presence of any reagent which would result in coupling of the two compounds. Such a reagent can be readily determined by those of ordinary skill in the art and can include, e.g., a base and/or an anhydride. Non-limiting examples of suitable bases include triethylamine, diisopropylethylamine, pyridine and dicyclohexylamine. Non-limiting examples of suitable anhydrides include acetic anhydride, acetic acid/benzylamine and toluene/benzylamine.

Compounds of formula (IV), including but not limited to compound having the requisite X and Y groups for preparing compounds of formula (I), (I-A), (I-B), (I-C), (I-D), (I-E), (I-F) and (I-G) as described therein, or any of their embodiments, as also described herein, can be prepared by

(a) reacting a compound of formula (II):

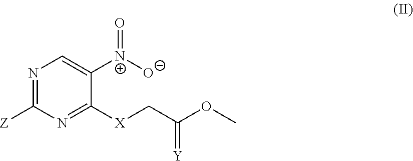

or a salt thereof, wherein Z is a halogen, with a compound of formula (V):

R²-AH (V)

or a salt thereof, to form a compound of formula

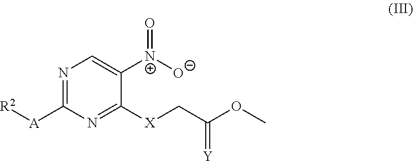

or a salt thereof; and

(b) reducing the resulting compound of formula (III) to form the compound of formula (IV), wherein Z is a halogen and X, Y and R² are as defined above.

In particular, a compound of formula (IV) may be prepared by reducing a compound of formula (III) with any suitable reducing agent proper to form a compound of formula (III). Suitable reducing agents can be readily determined by those of ordinary skill in the art and can include, e.g., sodium hydrosulfite, stannous chloride/hydrochloric acid and Zinc/acetic acid.

In addition, compounds of formula (I), including but not limited to the compounds of formula (I), (I-A), (I-B), (I-C), (I-D), (I-E), (I-F) and (I-G) as described herein, or any of their embodiments, as also described herein, can be prepared by

(a) reacting a compound of formula (II):

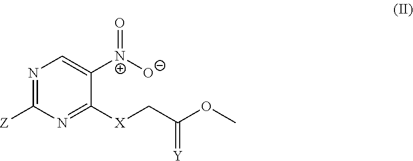

or a salt thereof, wherein Z is a halogen, with a compound of formula (V):

R²-AH (V)

or a salt thereof, to form a compound of formula (III):

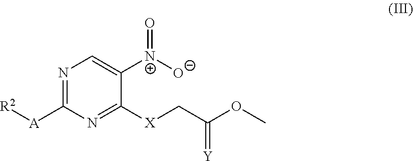

or a salt thereof; and

(b) reducing the compound of formula (III) to form a compound of formula (IV):

or a salt thereof; and

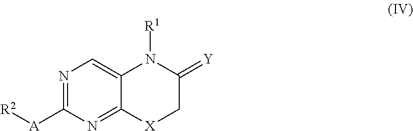

(c) reacting the compound of formula (IV) with a compound of formula (VI):

R³—C(═O)H (VI)

or a salt thereof, to form the compound of formula (I).

Compounds of formula (I), including, but not limited compounds having suitable X and Y groups to serve as intermediates in the synthesis of the compounds of formula (I), (I-A), (I-B), (I-C), (I-D), (I-E), (I-F) and (I-G) as described herein, or any of their embodiments, can be prepared by:

(a) reacting a compound of formula (VII):

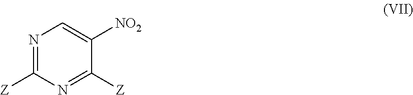

or a salt thereof, with a compound of formula (VIII):

HXCH₂CO₂CH₃ (VIII)

or a salt thereof, wherein X is as defined above and Z is halogen, to prepare the compound of formula (II).

In some embodiments, the compound of formula (VIII) is HSCH₂CO₂CH₃.

In particular, a compound of formula (II) may be prepared upon reaction of a compound of formula (VII) and formula (VIII) in the presence of any reagent which would achieve the desired product. Such a reagent can be readily determined by those of ordinary skill in the art and can include, e.g., a base. Non-limiting examples of suitable bases include triethylamine, diisopropylethylamine, pyridine, dicyclohexylamine, sodium hydroxide/ethanol and K₂CO₃/ethanol.

In some embodiments, a compound of formula (I) may be prepared by converting one compound of formula (I) to another compound of formula (I). For example, a compound of formula (I) can be converted to another compound of formula (I) as shown in Scheme 1.

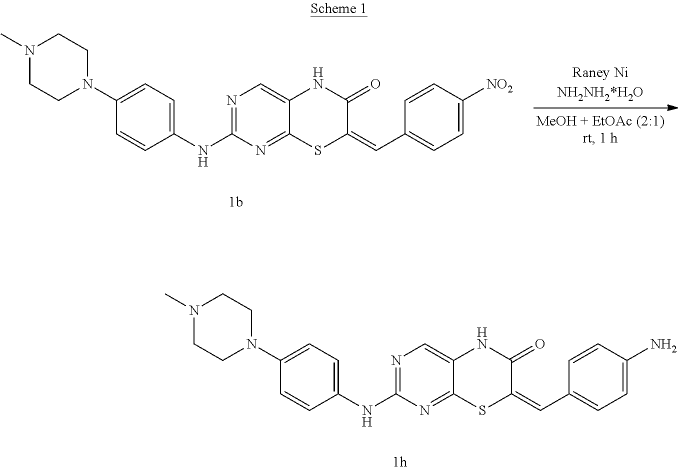 [SMILES: CN1CCN(c2ccc(Nc3ncc4c(n3)S/C(=C/c3ccc([N+](=O)[O-])cc3)C(=O)N4)cc2)CC1 ; CN1CCN(c2ccc(Nc3ncc4c(n3)S/C(=C/c3ccc(N)cc3)C(=O)N4)cc2)CC1]

A compound of formula (I), and other useful compounds and intermediates, can be formed as shown in Scheme 2. For example, a pyrimidine compound of formula (2) can be reacted with a hydrocarbyl of formula (5) to give a compound of formula (3). Reduction of the compound of formula (3) closes the thiomorpholino ring to give a compound of formula (4). Finally, reaction of the compound of formula (4) with the acetate of formula (6) can furnish the compound of formula (1).

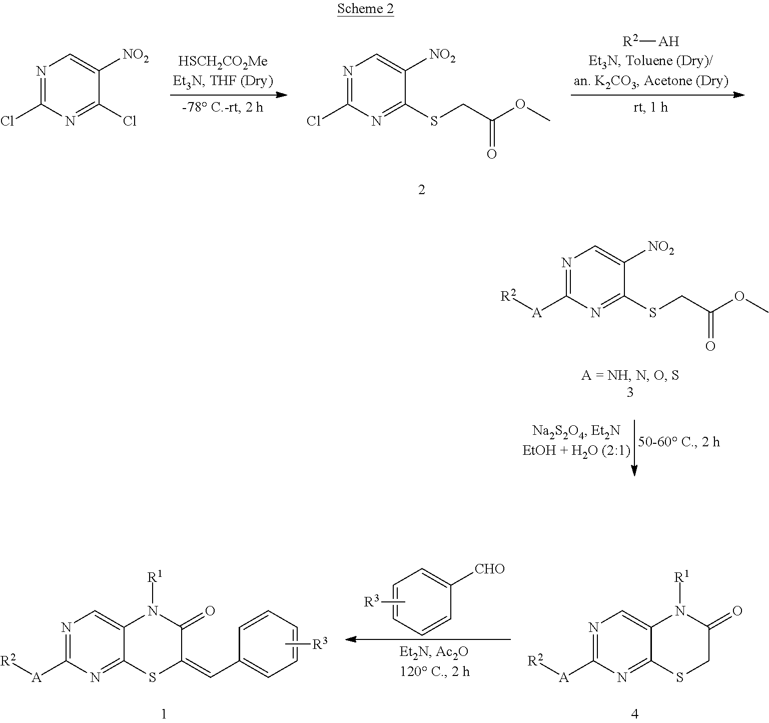

Starting materials, reagents and intermediates whose synthesis is not described herein are either commercially available, known in the literature, or may be prepared by methods known to one skilled in the art.

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn and C. W. Rees (Pergamon Press, 1996), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984) and Comprehensive Heterocyclic Chemistry II, Ed. A. R. Katritzky, C. W. Rees and E. F. V. Scriven (Pergamon Press, 1996).

### IV. PHARMACEUTICAL FORMULATIONS AND DOSAGE FORMS

When employed as pharmaceuticals, the compounds described herein can be administered in the form of pharmaceutical compositions, in which an active ingredient is combined with a pharmaceutically acceptable carrier. The active ingredient in such formulations may comprise from 0.1 to 99.99 weight percent. “Pharmaceutically acceptable carrier” means any carrier, diluent or excipient which is compatible with the other ingredients of the formulation and not deleterious to the recipient.

These compositions can be prepared in a manner well known in the pharmaceutical art and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or is intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners, and the like, may be necessary or desirable.

This disclosure also includes pharmaceutical compositions which contain, as the active ingredient, a compound as described herein or a pharmaceutically acceptable salt thereof, in combination with one or more pharmaceutically acceptable carriers (excipients). In some embodiments, the composition is suitable for topical administration. In making the compositions described herein, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier, or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, an active compound can be milled to provide the appropriate particle size prior to combining with the other ingredients. If an active compound is substantially insoluble, it can be milled to a particle size of less than 200 mesh. If an active compound is substantially water soluble, the particle size can be adjusted by milling to provide a substantially uniform distribution in the formulation, e.g. about 40 mesh.

The compounds described herein may be milled using known milling procedures such as wet milling to obtain a particle size appropriate for tablet formation and for other formulation types. Finely divided (nanoparticulate) preparations of the compounds described herein can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions described herein contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions described herein contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 300 to about 350, about 350 to about 400, or about 450 to about 500 mg of the active ingredient.

In some embodiments, the compositions described herein contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages of the compounds described herein may be used in the methods and uses described below.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound as described herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient.

The tablets or pills described herein can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerine monostearate, PEG-glycerine monostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2, or at least about 5 wt % of the compound as described herein. The topical formulations can be suitably packaged in tubes of, e.g., 100 g which are optionally associated with instructions for the treatment of the select indication.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound described herein can vary according to, e.g., the particular use for which the treatment is made, the manner of administration of the compound, the health and condition of the patient, and the judgment of the prescribing physician. The proportion or concentration of a compound in a pharmaceutical composition can vary depending upon a number of factors including dosage, chemical characteristics (e.g., hydrophobicity), and the route of administration. For example, the compounds as described herein can be provided in an aqueous physiological buffer solution containing about 0.1 to about 10% w/v of the compound for parenteral administration. Some typical dose ranges are from about 1 mg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. METHODS OF USE

Provided herein are methods of treating a cellular proliferative disorder in a patient. The method includes administering to the patient a therapeutically effective amount of a compound of formula (I), or a pharmaceutically acceptable salt thereof.

A “cellular proliferative disorder” means a disorder wherein cells are made by the body at an atypically accelerated rate. The expression “kinase-dependent proliferative disorder” refers to a proliferative disorder wherein the abnormally high cell proliferation is driven by the expression of a protein kinase.

A cellular proliferative disorder can include cancer. Non-limiting examples of cancers include bladder cancer, brain cancer, breast cancer, colorectal cancer, cervical cancer, gastrointestinal cancer, genitourinary cancer, head and neck cancer, lung cancer, ovarian cancer, prostate cancer, renal cancer, skin cancer and testicular cancer.

More particularly, cancers that may be treated by the compound, compositions and methods described herein include, but are not limited to, the following:

1) Breast cancers, including, e.g., ER⁺ breast cancer, ER⁻ breast cancer, her2⁻ breast cancer, her2⁺ breast cancer, stromal tumors such as fibroadenomas, phyllodes tumors and sarcomas and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, luminal B, basal A, basal B, and triple negative breast cancer, which is estrogen receptor negative (ER), progesterone receptor negative, and her2 negative (her2). In some embodiments, the breast cancer may have a high risk Oncotype score.

2) Cardiac cancers, including, e.g., sarcoma, e.g., angiosarcoma, fibrosarcoma, rhabdomyosarcoma, and liposarcoma; myxoma; rhabdomyoma; fibroma; lipoma and teratoma.

3) Lung cancers, including, e.g., bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma.

4) Gastrointestinal cancer, including, e.g., cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

5) Genitourinary tract cancers, including, e.g., cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, e.g., hepatoma, e.g., hepatocellular carcinoma; cholangiocarcinoma; hepatoblastoma; angiosarcoma; hepatocellular adenoma; and hemangioma.

7) Bone cancers, including, e.g., osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, e.g., cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma.

9) Gynecological cancers, including, e.g., cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma.

10) Hematologic cancers, including, e.g., cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenström's macroglobulinemia.

11) Skin cancers, including, e.g., malignant melanoma, basal cell carcinoma, squamous cell carcinoma, Kaposi's sarcoma, moles dysplastic nevi, lipoma, angioma, dermatofibroma, keloids, and psoriasis.

12) Adrenal gland cancers, including, e.g., neuroblastoma.

13) Pancreatic cancers, including, e.g., exocrine pancreatic cancers such as adenocarcinomas (M8140/3), adenosquamous carcinomas, signet ring cell carcinomas, hepatoid carcinomas, colloid carcinomas, undifferentiated carcinomas, and undifferentiated carcinomas with osteoclast-like giant cells; and exocrine pancreatic tumors.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer-using a compound of formula (I) may be combined with existing methods of treating cancers, e.g., by chemotherapy, irradiation, or surgery (e.g., oophorectomy). In some embodiments, a compound of formula (I) can be administered before, during, or after another anticancer agent or treatment.

A cellular proliferative disorder can also include hemangiomatosis in newborns, secondary progressive multiple sclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenoisis, and cirrhosis.

Also provided herein is a method of treating a neurological disorder in a patient. The method comprises administering to the patient a therapeutically effective amount of a compound as described herein or a pharmaceutically acceptable derivative thereof.

A neurological disorder can include Alzheimer's disease, Parkinson's disease, autism, enuresis, amyotrophic lateral sclerosis (ALS), hypoxia, hypoglycemia, epilepsy, Huntington's disease, multiple sclerosis, stroke and ischemia associated with stroke, neural paropathy, motor neuron diseases, sciatic crush, and peripheral neuropathy.

Treatment of the disorders as described herein may be accomplished through the inhibition of one or more kinases, e.g., ABL1, ABL2/ARG, PIK3-α, PIK3-β, PIK3-γ, PIK3-δ, c-Src, Fgr, and RIPK2, and mutants thereof. In some embodiments, the kinase is selected from ABL1 and PIK3-α. Accordingly, provided herein is a method of inhibiting one or more kinases in a patient, the method comprising administering to the patient a therapeutically effective amount of a compound of formula (I) or a pharmaceutically acceptable salt thereof.

In some embodiments, the methods described herein can be used in vitro, e.g., inhibiting one or more kinases in a cell, inhibiting cellular proliferation of cancer cells, inducing cell death of cancer cells, and inducing apoptosis of cancer cells. Such in vitro methods can be performed by contacting a cell (e.g., a cancer cell) with an effective amount of a compound of formula (I). Uses of such in vitro methods include, but are not limited to, use in a screening assay (e.g., wherein the compound is used as a positive control or standard compared to compounds of unknown activity or potency in inhibiting kinase activity).

### EXAMPLES

### Example 1

### Methyl 2-(2-chloro-5-nitropyrimidin-4-ylthio)acetate (2)

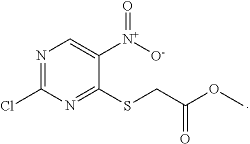 [SMILES: COC(=O)CSc1nc(Cl)ncc1[N+](=O)[O-]]

2,4-Dichloro-5-nitropyrimidine (5 g, 25.8 mmol) was dissolved in dry THF (60 mL) under nitrogen atmosphere and cooled the reaction mixture to −78° C. under dry ice. Methyl thioglycolate (2.3 mL, 25.8 mmol) was added by syringe and the reaction mixture was stirred at −78° C. for 10 min. Triethylamine (3.6 mL, 25.8 mmol) dissolved in dry THF (10 mL) was added drop-wise and the reaction was continued at −78° C. for 30 min and at room temperature for an additional hour. The reaction mixture was poured onto crushed ice (200 g), extracted with ethyl acetate (3×75 mL). The combined organic layer was washed with brine (150 mL) and dried over anhydrous Na₂SO₄. The salts were filtered and the solvent was removed under vacuum to give the crude compound, which was purified by column chromatography (Silica gel 60-200 mesh, 20% Ethyl Acetate/Hexane) to give the pure compound 2 (7 g) as light yellow viscous liquid; ¹H NMR: δ 9.23 (brs, 1H, C₆—H), 3.99 (s, 2H, S—CH₂—CO), 3.82 (s, 3H, OCH₃); LC-MS: 264.05 (M+1).

### Example 2

### General Procedure for the Preparation of Methyl 2-(2-Substituted-5-nitropyrimidin-4-ylthio)acetate (3)

### Method A:

The compound 2 (18.9 mmol) dissolved in dry toluene (100 mL) was added to triethylamine (2.6 mL, 18.9 mmol) at room temperature under nitrogen atmosphere and the reaction was stirred. After 5 min, the substituted aniline (18.9 mmol) was added to the above reaction and the stirring was continued for another 1 h. The solvent was reduced to half and the separated solid was filtered, washed with ether (50 mL) and with methanol (50 mL), and dried to get pure compound.

### Method B:

The substituted phenol/thiophenol (18.9 mmol) was dissolved in dry acetone (50 mL) under a nitrogen atmosphere. Anhydrous potassium carbonate (18.9 mmol) was added and the reaction was stirred for 10 min. Compound 2 (18.9 mmol) dissolved in dry acetone (50 mL) was added to the above stirred solution and the reaction was continued for an additional hour at room temperature. The crude reaction mixture was added slowly to crushed ice with vigorous stirring. The separated solid was filtered, washed with 50:50 ether/hexanes (50 mL), and dried to get pure compound.

### Example 3

### Methyl 2-(2-(4-(4-methylpiperazin-1-yl)phenylamino)-5-nitropyrimidin-4-ylthio) acetate (3a)

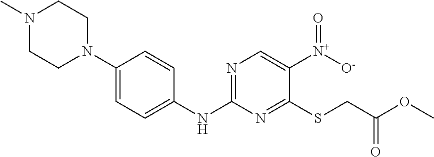

Compound 2 (5 g, 18.9 mmol) and 4-(4-methylpiperazino)aniline (3.6 g, 18.9 mmol) were treated in the presence of triethylamine (2.6 mL, 18.9 mmol) to prepare product 3a (7.8 g) according to Method A; m.p.: 188-190° C.; ¹H NMR: δ 10.72 (brs, 1H, NH), 9.10 (s, 1H, C₆—H), 7.44 (d, 2H, Ar—H, J=8.4 Hz), 6.98 (d, 2H, Ar—H, J=8.4 Hz), 4.06 (s, 2H, S—CH₂—CO), 3.44 (s, 3H, OCH₃), 3.38-3.36 (m, 4H, pip-H), 3.21-3.19 (m, 4H, pip-H), 2.73 (s, 3H, N—CH₃); LC-MS: 419.39 (M+1).

### Example 4

### Methyl 2-(5-nitro-2-(4-(pyridin-2-yl)piperazin-1-yl)pyrimidin-4-ylthio)acetate (3b)

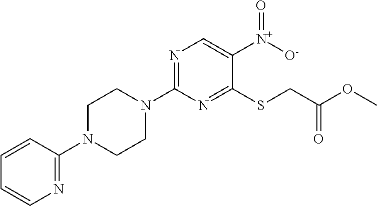 [SMILES: COC(=O)CSc1nc(N2CCN(c3ccccn3)CC2)ncc1[N+](=O)[O-]]

Compound 2 (2 g, 8.24 mmol) and 1-(2-pyridyl)piperazine (1.35 g, 8.24 mmol) were reacted in the presence of triethylamine (8.24 mmol) to afford compound 3b (2.5 g) according to Method A; m.p.: 199-202° C.; ¹H NMR: δ 9.05 (s, 1H, C₆—H), 8.22 (d, 1H, Ar—H, J=3.6 Hz), 7.56-7.55 (m, 1H, Ar—H), 6.71-6.70 (m, 2H, Ar—H), 4.12-4.07 (m, 4H, pip-H), 3.82 (s, 2H, S—CH₂—CO), 3.74 (s, 3H, OCH₃), 3.70-3.67 (m, 4H, pip-H). LC-MS: 391.14 (M+1).

### Example 5

### Methyl 2-(5-nitro-2-(4-(piperazin-1-yl)phenoxy)pyrimidin-4-ylthio)acetate (3c)

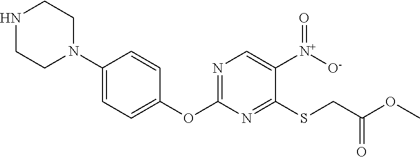 [SMILES: COC(=O)CSc1nc(Oc2ccc(N3CCNCC3)cc2)ncc1[N+](=O)[O-]]

Compound 2 (1.5 g, 5.7 mmol) and 4-(1-piperazinyl)phenol (1.01 g, 5.7 mmol) were treated in the presence of anhydrous K₂CO₃ to prepare product 3c (1.9 g) according to Method B; m.p.: 193-195° C.; ¹H NMR: δ 9.08 (brs, 1H, NH), 8.92 (s, 1H, C₆—H), 6.88 (d, 2H, Ar—H, J=8.7 Hz), 6.69 (d, 2H, Ar—H, J=8.8 Hz), 4.06 (s, 2H, S—CH₂—CO), 4.05-4.03 (m, 4H, pip-H), 3.69 (s, 3H, OCH₃), 3.09-3.07 (m, 4H, pip-H); LC-MS: 406.19 (M+1).

### Example 6

### Methyl 2-(2-(4-chlorophenylthio)-5-nitropyrimidin-4-ylthio)acetate (3d)

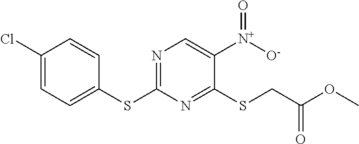 [SMILES: COC(=O)CSc1nc(Sc2ccc(Cl)cc2)ncc1[N+](=O)[O-]]

Compound 2 (5 g, 18.9 mmol) and 4-chlorothiophenol (2.74 g, 18.9 mmol) were reacted in the presence of anhydrous K₂CO₃ (2.62 g, 18.9 mmol) to prepare compound 3d (3.8 g) according to the Method B; m.p.: 157-159° C.; ¹H NMR: δ 9.25 (s, 1H, C₆—H), 7.64-7.60 (m, 4H, Ar—H), 3.65 (s, 2H, S—CH₂—CO), 3.59 (s, 3H, OCH₃); LC-MS: 372.06 (M+1).

### Example 7

### Methyl 2-(2-(2,6-dichlorobenzylthio)-5-nitropyrimidin-4-ylthio)acetate (3e)

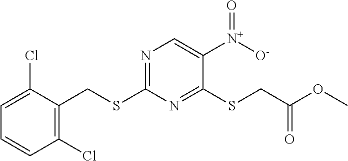 [SMILES: COC(=O)CSc1nc(SCc2c(Cl)cccc2Cl)ncc1[N+](=O)[O-]]

Compound 2 (2 g, 7.6 mmol) and 2,6-dichlorobenzylthiol (1.47 g, 7.6 mmol) were reacted with anhydrous K₂CO₃ (1.05 g, 7.6 mmol) to prepare compound 3e (1.8 g) according to the Method B; m.p.: 155-158° C.; ¹H NMR: δ 9.26 (s, 1H, C₆—H), 7.58 (d, 2H, Ar—H, J=8.4 Hz), 7.44 (t, 1H, Ar—H, J=8.4 & 8.1 Hz), 4.76 (s, 2H, Ar—CH₂), 4.21 (s, 2H, S—CH₂—CO), 3.65 (s, 3H, OCH₃); LC-MS: 419.97 (M+1).

### Example 8

### Methyl 2-((5-nitro-2-(4-(pyrimidin-2-yl)piperazin-1-yl)pyrimidin-4-yl)thio)acetate (3f)

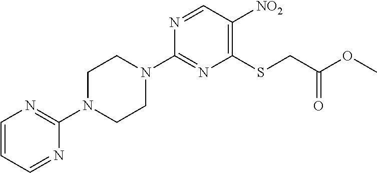 [SMILES: COC(=O)CSc1nc(N2CCN(c3ncccn3)CC2)ncc1[N+](=O)[O-]]

Compound 2 (6.0 g, 22.8 mmol) and 1-(2-pyrimidyl)piperazine (3.74 g, 22.8 mmol) were reacted in the presence of triethylamine (3.18 mL, 22.8 mmol) to provide compound 3f (7.2 g) according to Method A. m.p.: 250-252° C.; ¹H NMR: δ 10.41 (brs, 1H, NH), 8.42-8.40 (m, 2H, Ar—H), 7.90 (s, 1H, C₄-H), 6.65-6.64 (m, 1H, Ar—H), 4.10 (s, 2H, S—CH₂—CO) 3.80-3.79 (m, 4H, pip-H), 3.70-3.68 (m, 4H, pip-H), 3.56 (s, 3H, OCH₃); LC-MS: 392.11 (M+1).

### Example 9

### Methyl 2-((2-((4-morpholinophenyl)amino)-5-nitropyrimidin-4-yl)thio)acetate (3 g)

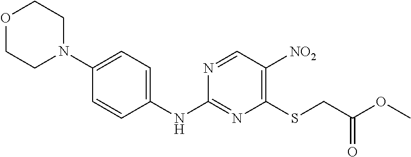 [SMILES: COC(=O)CSc1nc(Nc2ccc(N3CCOCC3)cc2)ncc1[N+](=O)[O-]]

Compound 2 (6.0 g, 22.8 mmol) and 4-morpholinoaniline (4.06 g, 22.8 mmol) were reacted in the presence of triethylamine (3.18 mL, 22.8 mmol) to provide compound 3 g (7.7 g) according to Method A; m.p.: 188-190° C.; ¹H NMR: δ 10.69 (brs, 1H, NH), 9.11 (s, 1H, C₄—H), 7.44 (d, 2H, Ar—H, J=7.7 Hz), 6.94 (d, 2H, Ar—H, J=7.8 Hz), 4.07 (s, 2H, S—CH₂—CO), 3.77-3.75 (m, 4H, morp-H), 3.46 (s, 3H, OCH₃), 3.12-3.10 (m, 4H, morp-H); LC-MS: 406.23 (M+1).

### Example 10

### Methyl 2-((2-((4-(4-methylpiperidin-1-yl)phenyl)amino)-5-nitropyrimidin-4-yl)thio)acetate (3h)

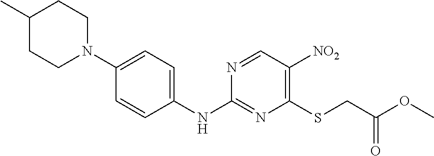

Compound 2 (1.32 g, 5.0 mmol) and 4-(4-methylpiperidin-1-yl)-phenylamine (950 mg, 5.0 mmol) were reacted in the presence of triethylamine (700 μL, 5.0 mmol) to afford compound 3h (1.8 g) according to Method A; m.p.: 180-184° C.; ¹H NMR: δ 10.66 (brs, 1H, NH), 9.10 (s, 1H, C₄—H), 7.40 (d, 2H, Ar—H, J=8.5 Hz), 6.92 (d, 2H, Ar—H, J=8.5 Hz), 4.23 (s, 2H, S—CH₂—CO), 3.68-3.65 (m, 2H, pip-H), 3.46 (s, 3H, OCH₃), 2.68-2.65 (m, 2H, pip-H), 1.73-1.71 (m, 2H, pip-H), 1.54-1.50 (m, 1H, pip-H), 1.28-1.25 (m, 2H, pip-H), 0.96 (d, 3H, CH—CH₃, J=6.5 Hz); LC-MS: 418.24 (M+1).

### Example 11

### General Procedure for the Preparation of 2-(substituted)-5H-pyrimido[4,5-b][1,4]thiazin-6(7H)-one (4)

Sodium hydrosulfite (12 mmol) was added portion wise (in 4 portions for every 5 min.) to a stirred solution of 3 (6 mmol) in ethanol and water (2:1) and triethylamine (24 mmol). The temperature of the reaction was kept at 50° C. upon the addition and the reaction was continued at 60° C. for another 1.5 h. then cooled to room temperature. The reaction mixture was poured into crushed ice (300 g) and extracted with chloroform (3×75 mL). The combined organic layers were washed with brine (150 mL), dried with anhydrous Na₂SO₄ (10 g), and evaporated in vacuo. The obtained crude product was treated with 2% methanol/ether. The solid was filtered, washed with ether (20 mL), and dried to get pure product 4.

### Example 12

### 2-(4-(4-Methylpiperazin-1-yl)phenylamino)-5H-pyrimido[4,5-b][1,4]thiazin-6(7)-one (4a)

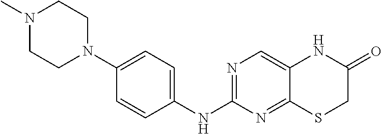 [SMILES: CN1CCN(c2ccc(Nc3ncc4c(n3)SCC(=O)N4)cc2)CC1]

Following the general procedure described in Example 8, sodium hydrosulfite (2.09 g, 12 mmol) was added to a stirred solution of compound 3a (2.5 g, 6 mmol) in ethanol (50 mL), water (25 mL) and triethylamine (3.35 mL, 24 mmol) to obtain the compound 4a (0.6 g). m.p.: 272-274° C.; ¹H NMR: δ 10.45 (brs, 1H, NH), 9.34 (brs, 1H, NH), 7.89 (s, 1H, C₄—H), 7.49 (d, 2H, Ar—H, J=8.4 Hz), 6.86 (d, 2H, Ar—H, J=8.4 Hz), 3.69 (s, 2H, C₄—H), 3.05-3.03 (m, 4H, pip-H), 2.46-2.44 (m, 4H, pip-H), 2.22 (s, 3H, N—CH₃); LC-MS: 357.28 (M+1).

### Example 13

### 2-(4-(Pyridin-2-yl)piperazin-1-yl)-5H-pyrimido[4,5-b][1,4]thiazin-6(7H)-one (4b)

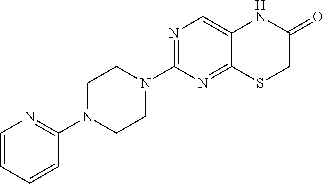 [SMILES: O=C1CSc2nc(N3CCN(c4ccccn4)CC3)ncc2N1]

Reductive cyclization of compound 3b (2 g, 5.1 mmol) with sodium hydrosulfite (1.78 g, 10.2 mmol) in ethanol (40 mL), water (20 mL), and triethylamine (2.84 mL, 20.4 mmol) gave the compound 4b (0.41 g); m.p.: 263-265° C.; ¹H NMR: δ 10.36 (brs, 1H, NH), 8.11 (d, 1H, Ar—H, J=8.5 Hz), 7.88 (s, 1H, C₄—H), 7.53-7.52 (m, 1H, Ar—H), 6.84 (d, 1H, Ar—H, J=8.5 Hz), 6.65-6.63 (m, 1H, Ar—H), 3.74-3.71 (m, 4H, pip-H), 3.64 (s, 2H, C₄—H), 3.55-3.52 (m, 4H, pip-H); LC-MS: 329.16 (M+1).

### Example 14

### 2-(4-(piperazin-1-yl)phenoxy)-5H-pyrimido[4,5-b][1,4]thiazin-6(7H)-one (4c)

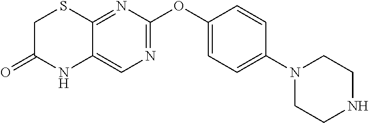 [SMILES: O=C1CSc2nc(Oc3ccc(N4CCNCC4)cc3)ncc2N1]

Sodium hydrosulfite (1.03 g, 5.9 mmol) was added to a stirred solution of compound 3c (1.2 g, 2.9 mmol) in ethanol (20 mL), water (10 mL) and triethylamine (1.65 mL, 11.8 mmol) to prepare pure compound 4c (0.3 g); m.p.: 309-312° C.; ¹H NMR: δ 10.39 (brs, 1H, NH), 8.88 (brs, 1H, NH), 7.91 (s, 1H, C₄—H), 6.84 (d, 2H, Ar—H, J=8.6 Hz), 6.68 (d, 2H, Ar—H, J=8.6 Hz), 3.78-3.76 (m, 4H, pip-H), 3.68 (s, 2H, C₇—H), 3.01-2.99 (m, 4H, pip-H); LC-MS: 344.25 (M+1).

### Example 15

### 2-(2,6-Dichlorobenzylthio)-5H-pyrimido[4,5-b][1,4]thiazin-6(7H)-one (4d)

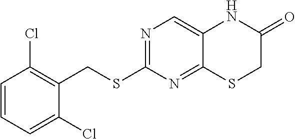 [SMILES: O=C1CSc2nc(SCc3c(Cl)cccc3Cl)ncc2N1]

Reductive cyclization of compound 3e (1.5 g, 3.5 mmol) in ethanol (30 mL), water (15 mL), triethylamine (3.48 mL, 25 mmol), and sodium hydrosulfite (2.17 g, 12.5 mmol) afforded pure compound 4d (0.4 g); m.p.: 242-244° C.; ¹H NMR: δ 10.80 (brs, 1H, NH), 8.09 (s, 1H, C₄—H), 7.53 (d, 2H, Ar—H, J=7.8 Hz), 7.38 (t, 1H, Ar—H, J=7.8 & 8.4 Hz), 4.65 (s, 2H, Ar—CH₂), 3.79 (s, 2H, C—H); LC-MS: 359.98 (M+1).

### Example 16

### 2-(4-(Pyrimidin-2-yl)piperazin-1-yl)-5H-pyrimido[4,5-b][1,4]thiazin-6(7H)-one (4e)

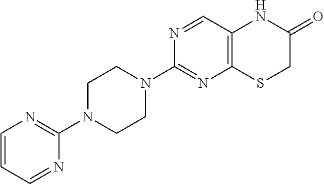 [SMILES: O=C1CSc2nc(N3CCN(c4ncccn4)CC3)ncc2N1]

Sodium hydrosulfite (6.69 g, 25.6 mmol) was added portion wise (in 4 portions for every 5 min) to a stirred solution of compound 3f (5.0 g, 12.8 mmol) in ethanol (100 mL), water (50 mL) and triethylamine (7.1 mL, 51.2 mmol). The temperature of the reaction was kept at 50° C. during the addition and the reaction was continued at 60° C. for another 1.5 h. The reaction mixture was then cooled to room temperature and was then poured onto crushed ice (300 g). The resulting separated solid was collected by filtration, washed with ether (50 mL) and methanol (10 mL), and then dried to afford analytically pure compound 4e (1.8 g); m.p.: 314-3160C; ¹H NMR: δ 10.40 (brs, 1H, NH), 8.41-8.40 (m, 2H, Ar—H), 7.92 (s, 1H, C₄—H), 6.68-6.67 (m, 1H, is Ar—H), 3.82-3.81 (m, 4H, pip-H), 3.75-3.74 (m, 4H, pip-H), 3.70 (s, 2H, C₇—H); LC-MS: 330.17 (M+1).

### Example 17

### 2-((4-Morpholinophenyl)amino)-5H-pyrimido[4,5-b][1,4]thiazin-6(7H)-one (4f)

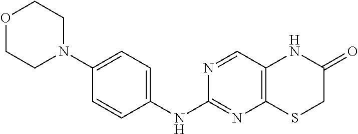 [SMILES: O=C1CSc2nc(Nc3ccc(N4CCOCC4)cc3)ncc2N1]

Sodium hydrosulfite (4.29 g, 24.6 mmol) was added portion wise (in 4 portions every 5 min) to a stirred solution of compound 3g (5.0 g, 12.3 mmol) in ethanol (100 mL), water (50 mL) and triethylamine (6.85 mL, 49.2 mmol). The temperature of the reaction was kept at 50° C. during the addition and the reaction was continued at 60° C. for another 2 h. The reaction mixture was then cooled to room temperature and was then poured onto crushed ice (300 g). The resulting separated solid was collected by filtration, washed with 2% MeOH/Ether (20 mL) and then dried to afford analytically pure compound 4f (1.7 g); m.p.: 288-290° C.; ¹H NMR: δ 10.46 (brs, 1H, NH), 9.36 (brs, 1H, NH), 7.92 (s, 1H, C₄—H), 7.53 (d, 2H, Ar—H, J=8.0 Hz), 6.89 (d, 2H, Ar—H, J=8.0 Hz), 3.75-3.74 (m, 4H, morp-H), 3.71 (s, 21H, C₄—H), 3.03-3.01 (m, 4H, morp-H); LC-MS: 344.20 (M+1).

### Example 18

### 2-((4-(4-Methylpiperidin-1-yl)phenyl)amino)-5H-pyrimido[4,5-b][1,4]thiazin-6(7H)-one (4g)

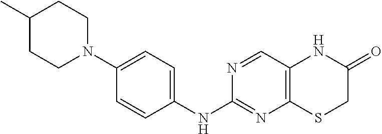 [SMILES: CC1CCN(c2ccc(Nc3ncc4c(n3)SCC(=O)N4)cc2)CC1]

Sodium hydrosulfite (1.25 g, 7.2 mmol) was added portion wise (in 4 portions every 5 min.) to a stirred solution of compound 3h (1.5 g, 3.6 mmol) in ethanol (30 mL), water (15 mL) and triethylamine (2.0 mL, 14.4 mmol). The temperature of the reaction was kept at 50° C. during the addition and the reaction was continued at 60° C. for another 2 h. then cooled to room temperature. The reaction mixture was then cooled to room temperature and was then poured is 5 onto crushed ice (300 g). The resulting separated solid was collected by filtration, washed with 2% MeOH/Ether (10 mL) and dried to afford analytically pure compound 4g (580 mg); m.p.: 278-280° C.; ¹H NMR: δ 10.45 (brs, 1H, NH), 9.32 (brs, 1H, NH), 7.91 (s, 1H, C₄—H), 7.49 (d, 2H, Ar—H, J=8.0 Hz), 6.88 (d, 2H, Ar—H, J=8.0 Hz), 3.71 (s, 2H, C₄—H), 3.55-3.54 (m, 2H, pip-H), 2.59-2.56 (m, 2H, pip-H), 1.70-1.69 (m, 2H, pip-H), 1.46-1.45 (m, 1H, pip-H), 1.26-1.25 (m, 2H, pip-H), 0.95 (d, 3H, CH—CH₃, J=6.4 Hz); LC-MS: 356.36 (M+1).

### Example 19

### General Procedure for the Preparation of 2-(substituted)-7-(arylidene)-5H-pyrimido[4,5-b][1,4]thiazin-6(7H)-one (1)

A solution of 4 (1 mmol); aldehyde (1 mmol), triethylamine (4 mmol) and acetic anhydride (10 mL) were heated to 120° C. under nitrogen atmosphere and the reaction was continued for 2 h. The reaction mixture was allowed to cool to room temperature. The solvent was removed and purified by column chromatography to afford pure product 5. In some cases, the reaction mixture was allowed to cool to room temperature, the solid was isolated by filtration, washed with ether and dried to get pure product 1.

### Example 20

### (E)-7-(4-Fluorobenzylidene)-2-(4-(4-methylpiperazin-1-yl)phenylamino)-5H-pyrimido[4,5-b][1,4]thiazin-6(7H)-one (1a)

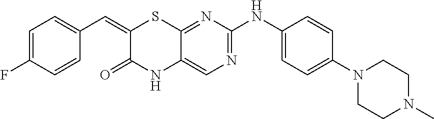 [SMILES: CN1CCN(c2ccc(Nc3ncc4c(n3)S/C(=C/c3ccc(F)cc3)C(=O)N4)cc2)CC1]

Following the general procedure detailed in Example 13, compound 4a (500 mg, 1.4 mmol) was condensed with 4-fluorobenzaldehyde (208 mg, 1.4 mmol) and resulted in the formation of crude 1a which was purified by column chromatography (basic alumina 50-200 μm, 2-5% MeOH/DCM) to afford pure 1a (120 mg); m.p.: 172-175° C.; ¹H NMR: δ 10.99 (brs, 1H, NH), 9.39 (brs, 1H, NH), 8.00 (s, 1H, ═CH), 7.86 (s, 1H, C₄—H), 7.72 (d, 2H, Ar—H, J=5.4 Hz), 7.47 (d, 2H, Ar—H, J=8.4 Hz), 7.36 (d, 2H, Ar—H, J=8.4 Hz), 6.88 (d, 2H, Ar—H, J=9.0 Hz), 3.06-3.03 (m, 4H, pip-H), 2.48-2.46 (m, 4H, pip-H), 2.23 (s, 3H, N—CH₃); LC-MS: 463.30 (M+1).

### Example 21

### (E)-2-(4-(4-Methylpiperazin-1-yl)phenylamino)-7-(4-nitrobenzylidene)-5H-pyrimido[4,5-b][1,4]thiazin-6(7H)-one (1b)

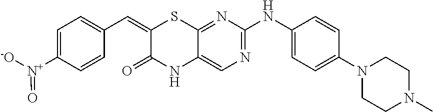

4-Nitrobenzaldehyde (212 mg, 1.4 mmol) was condensed with compound 4a (500 mg, 1.4 mmol) in the presence of triethylamine (780 μl, 1.4 mmol) in acetic anhydride (10 mL). The crude compound was purified by column chromatography (basic alumina 50-200 m, 5% MeOH/DCM) to afford pure 1b (260 mg); m.p.: 276-279° C. ¹H NMR: δ 11.16 (brs, 1H, NH), 9.42 (brs, 1H, NH), 8.33 (d, 2H, Ar—H, J=7.8 Hz), 8.03 (s, 1H, —CH), 7.93 (s, 1H, C₄—H), 7.91 (d, 2H, Ar—H, J=8.4 Hz), 7.45 (d, 2H, Ar—H, J=8.4 Hz), 6.87 (d, 2H, Ar—H, J=9.0 Hz), 3.06-3.04 (m, 4H, pip-H), 2.46-2.43 (m, 4H, pip-H), 2.22 (s, 3H, N—CH₃); LC-MS: 490.16 (M+1).

### Example 22

### (E)-4-((2-(4-(4-Methylpiperazin-1-yl)phenylamino)-6-oxo-5H-pyrimido[4,5-b][1,4]thiazin-7(6H)-ylidene)methyl)phenyl acetate (1c)

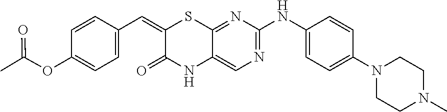 [SMILES: CC(=O)Oc1ccc(/C=C2/Sc3nc(Nc4ccc(N5CCN(C)CC5)cc4)ncc3NC2=O)cc1]

A mixture of compound 4a (500 mg, 1.4 mmol) and 4-hydroxybenzaldehyde (171 mg, 1.4 mmol) in the presence of triethylamine (780 μl, 1.4 mmol) in acetic anhydride (10 mL) was heated at 120° C. for 2 h. Removal of the solvent and purification of the crude product by column chromatography (basic alumina 50-200 μm, 2% MeOH/DCM) afforded pure 1c (80 mg); m.p.: 168-171° C.; ¹H NMR: δ 11.00 (brs, 1H, NH), 9.39 (brs, 1H, NH), 8.21 (s, 1H, ═CH), 8.01 (s, 1H, C₄—H), 7.71 (d, 2H, Ar—H, J=8.4 Hz), 7.47 (d, 2H, Ar—H, J=9.0 Hz), 6.92 (d, 2H, Ar—H, J=8.4 Hz), 6.88 (d, 2H, Ar—H, J=8.4 Hz), 3.14-3.06 (m, 4H, pip-H), 2.48-2.44 (m, 4H, pip-H), 2.23 (s, 3H, N—CH₃), 2.10 (s, 3H, OCOCH₃); LC-MS: 503.20 (M+1).

### Example 23

### (E)-N-(7-(4-Methoxy-3-nitrobenzylidene)-6-ozo-6,7-dihydro-5H-pyrimido[4,5-b][1,4]thiazin-2-yl)-N-(4-(4-methylpiperazin-1-yl)phenyl)acetamide (1d)

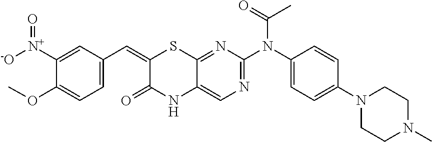

4-Methoxy-3-nitrobenzaldehyde (254 mg, 1.4 mmol), compound 4a (500 mg, 1.4 mmol), triethylamine (780 μl, 1.4 mmol) and acetic anhydride (10 mL) was heated to 120° C. for 2 h. The solvent was then removed and the crude compound was purified by column chromatography (basic alumina 50-200 μm, 5% Methanol/DCM) to afford pure 1d (110 mg); m.p.: 174-176° C.; ¹H NMR: δ 11.37 (brs, 1H, NH), 8.22 (s, 1H, ═CH), 8.20 (s, 1H, C₄—H), 7.97 (d, 1H, Ar—H, J=7.8 Hz), 7.87 (s, 1H, Ar—H), 7.50 (d, 1H, Ar—H, J=7.8 Hz), 7.06 (d, 2H, Ar—H, J=8.4 Hz), 6.93 (d, 2H, Ar—H, J=8.4 Hz), 3.99 (s, 3H, OCH₃), 3.16-3.14 (m, 4H, pip-H), 2.48-2.46 (m, 4H, pip-H), 2.24 (s, 3H, N—CH₃), 2.09 (s, 3H, NCOCH₃); LC-MS: 562.31 (M+1).

### Example 24

### (E)-7-(4-Nitrobenzylidene)-2-(4-(pyridin-2-yl)piperazin-1-yl)-5H-pyrimido[4,5-b][1,4]thiazin-6(7H)-one (1e)

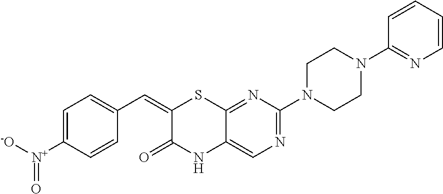

4-Nitrobenzaldehyde (230 mg, 1.5 mmol) and compound 4b (500 mg, 1.5 mmol) were condensed in the presence of triethylamine (840 μl, 1.5 mmol) in acetic anhydride (10 mL). The reaction was allowed to attain room temperature, the solid was then filtered, washed with ether (20 mL) and then methanol (5 mL), and dried to get pure le (240 mg); m.p.: 332-334° C.; ¹H NMR: δ 11.08 (brs, 1H, NH), 8.32 (d, 2H, Ar—H, J=6.9 Hz), 8.11-8.09 (m, 1H, Ar—H), 8.03 (s, 1H, ═CH), 7.92-7.89 (m, 3H, C₄—H & Ar—H), 7.53 (d, 1H, Ar—H, J=7.5 Hz), 6.84 (d, 1H, Ar—H, J=7.5 Hz), 6.64-6.63 (m, 1H, Ar—H), 3.74-3.71 (m, 4H, pip-H), 3.55-3.53 (m, 4H, pip-H); LC-MS: 462.34 (M+1).

### Example 25

### (E)-2-(4-(4-Acetylpiperazin-1-yl)phenoxy)-7-(4-nitrobenzylidene)-5H-pyrimido[4,5-b][1,4]thiazin-6(7H)-one (10f)

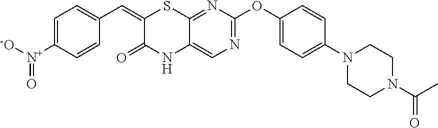

Compound 4c (500 g, 1.5 mmol) was condensed with 4-nitrobenzaldehyde (220 mg, 1.5 mmol) in triethylamine (800 μl, 1.5 mmol) and acetic anhydride (10 mL) at 120° C. The reaction mixture was cooled to room temperature, ether (2 mL) was added and the mixture was stirred for 10 min. The separated solid was isolated by filtration, washed with ether (50 mL), and dried to afford pure 1f (295 mg); m.p.: 302-304° C.; ¹H NMR: δ 11.12 (brs, 1H, NH), 8.34-8.33 (m, 2H, Ar—H), 8.06 (s, 1H, ═CH), 7.94-7.92 (m, 3H, C₄—H & Ar—H), 7.00-6.97 (m, 4H, Ar—H), 3.79-3.77 (m, 4H, pip-H), 3.18-3.16 (m, 4H, pip-H), 2.24 (s, 3H, NCOCH₃); LC-MS: 519.23 (M+1).

### Example 26

### (E)-N-(7-((1-Acetyl-1H-indol-3-yl)methylene)-6-oxo-6,7-dihydro-5H-pyrimido[4,5-b][1,4]thiazin-2-yl)-N-(4-(4-methylpiperazin-1-yl)phenyl)acetamide (1 g)

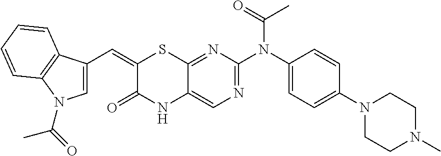 [SMILES: CC(=O)N(c1ccc(N2CCN(C)CC2)cc1)c1ncc2c(n1)S/C(=C/c1cn(C(C)=O)c3ccccc13)C(=O)N2]

A solution of compound 4a (500 mg, 1.4 mmol), indole-3-carboxaldehyde (204 mg, 1.4 mmol), triethylamine (780 μl, 1.4 mmol) and acetic anhydride (10 mL) was heated to 120° C. The reaction was then cooled to room temperature and ether (5 mL) was added slowly with stirring. The solid was then isolated by filtration, washed with ether (50 mL), and dried to get pure 1 g (120 mg); m.p.: 289-292° C.; ¹H NMR: δ 11.40 (brs, 1H, NH), 8.40 (d, 1H, Ar—H, J=82 Hz), 8.27 (s, 1H, Ar—H), 8.13 (s, 1H, ═CH), 8.08 (s, 1H, C₄—H), 7.86 (d, 1H, Ar—H, J=7.7 Hz), 7.47 (t, 1H, Ar—H, J=7.7 & 7.6 Hz), 7.41 (t, 1H, Ar—H, J=7.5 & 7.4 Hz), 7.10 (d, 2H, Ar—H, J=8.4 Hz), 6.95 (d, 2H, Ar—H, J=8.5 Hz), 3.16-3.14 (m, 4H, pip-H), 2.82 (s, 3H, NCOCH₃), 2.46-2.44 (m, 4H, pip-H), 2.23 (s, 3H, NCH₃), 2.10 (s, 3H, NCOCH₃); LC-MS: 568.32 (M+1).

### Example 27

### (E)-7-(4-Aminobenzlidene)-2-(4-(4-methylpiperazin-1-yl)phenylamino)-5H-pyrimido[4,5-b][1,4]thiazin-6(7H)-one (1b)

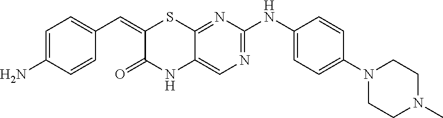 [SMILES: CN1CCN(c2ccc(Nc3ncc4c(n3)S/C(=C/c3ccc(N)cc3)C(=O)N4)cc2)CC1]

Raney nickel (100 mg) was added to a solution of compound 1b (200 mg, 0.4 mmol), absolute ethanol (20 mL) and ethyl acetate (10 mL) with vigorous stirring. Hydrazine hydrate (1 mL) in ethanol (5 mL) was added drop-wise to the above reaction mixture at room temperature and the stirring was continued for 1 h. Dichloromethane (50 mL) and water (50 mL) were added to the reaction mixture and the reaction was stirred for another 30 min. The reaction mixture was then filtered through celite. The celite pad was washed thoroughly with dichloromethane (100 mL), the organic layer was then separated and the aqueous layer was extracted with dichloromethane (50 mL). The combined organic layers were washed thoroughly with brine (3×100 mL), dried over anhydrous Na₂SO₄ (5 g), and the solvent was removed under vacuum. The crude compound was purified by column chromatography (basic alumina 50-200 μm, 8% MeOH+DCM) to afford pure 1 h (40 mg); m.p.: 264-266° C.; ¹H NMR: δ 10.66 (brs, 1H, NH), 9.33 (brs, 1H, NH), 7.94 (s, 1H, ═CH), 7.68 (s, 1H, C₄—H), 7.47 (d, 2H, Ar—H, J=8.4 Hz), 7.40 (d, 2H, Ar—H, J=8.4 Hz), 6.89 (d, 2H, Ar—H, J=9.0 Hz), 6.66 (d, 2H, Ar—H, J=8.4 Hz), 5.86 (brs, 2H, NH2), 3.06-3.03 (m, 4H, pip-H), 2.48-2.46 (m, 4H, pip-H), 222 (s, 3H, N—CH₃); LC-MS: 460.19 (M+1).

### Example 28

### (E)-4-((6-Oxo-2-(4-(pyridin-2-yl)piperazin-1-yl)-5H-pyrimido[4,5-b][1,4]thiazin-7(6H)-ylidene)methyl)phenyl acetate (1i)

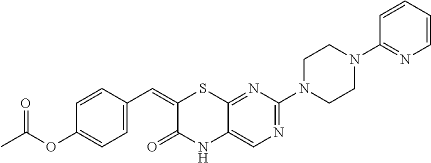 [SMILES: CC(=O)Oc1ccc(/C=C2/Sc3nc(N4CCN(c5ccccn5)CC4)ncc3NC2=O)cc1]

A solution of compound 4b (500 mg, 1.5 mmol), 4-hydroxybenzaldehyde (183 mg, 1.5 mmol), triethylamine (840 μL, 6.0 mmol) and acetic anhydride (10 mL) was heated to 120° C. The reaction mixture was then cooled room temperature, the separated solid was collected by filtration, washed with ether (20 mL) and dried to get pure compound 11 (160 mg); m.p.: 276-278° C.; ¹H NMR: δ 10.94 (brs, 1H, NH), 8.15 (s, 1H, ═CH), 8.04 (s, 1H, C₄—H), 7.87 (s, 1H, Ar—H), 7.73 (d, 2H, Ar—H, J=6.8 Hz), 7.57 (t, 1H, Ar—H), 7.31 (d, 2H, Ar—H, J=6.9 Hz), 6.88 (d, 1H, Ar—H, J=7.6 Hz), 6.68 (t, 1H, Ar—H), 3.77-3.75 (m, 4H, pip-H), 3.59-3.57 (m, 4H, pip-H), 2.32 (s, 3H, OCOCH₃); LC-MS: 475.12 (M+1).

### Example 29

### (E)-7-(4-Nitrobenzylidene)-2-(4-(pyrimidin-2-yl)piperazin-1-yl)-5H-pyrimido[4,5-b][1,4]thiazin-6(7H)-one (1j)

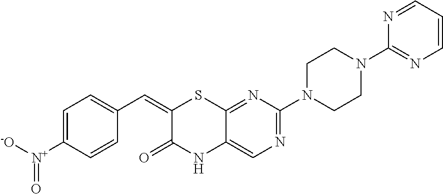

Compound 4e (500 mg, 1.5 mmol) was condensed with 4-nitrobenzaldehyde (230 mg, 1.5 mmol) in the presence of triethylamine (850 μL, 6.0 mmol) in acetic anhydride (10 mL) at 120° C. The reaction mixture was cooled to room temperature, filtered the separated solid, washed with methanol (10 mL) and finally with ether (20 mL). The crude product was purified by column chromatography (Basic alumina 50-200 μm, 10% MeOH/CHCl₃) to get pure compound 1j (220 mg); m.p: 320° C. (Decomp.); ¹H NMR: δ 11.13 (brs, 1H, NH), 8.41-8.37 (m, 4H, Ar—H), 8.07 (s, 1H, ═CH), 7.96-7.94 (m, 3H, C₄—H & Ar—H), 6.68 (s, 1H, Ar—H), 3.83-3.80 (m, 4H, pip-H), 3.76-3.74 (m, 4H, pip-H); LC-MS: 463.09 (M+1).

### Example 30

### (E)-2-((4-Morpholinophenyl)amino)-7-(4-nitrobenzylidene)-5H-pyrimido[4,5-b][1,4]thiazin-6(7H)-one (1k)

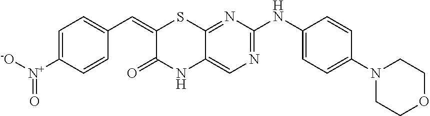

4-nitrobenzaldehyde (220 mg, 1.45 mmol), compound 4f (500 mg, 1.45 mmol), triethylamine (810 μL, 5.8 mmol) and acetic anhydride (10 mL) were heated to 120° C. for 2 h. The solvent was removed and the crude compound was purified by column chromatography is 5 (Basic alumina 50-200 μm, 5% MeOH/DCM) to give pure compound 1k (160 mg); m.p.: 302-304° C.; ¹H NMR: δ 11.17 (brs, 1H, NH), 9.45 (brs, 1H, NH), 8.36 (d, 2H, Ar—H, J=8.8 Hz), 8.06 (s, 1H, ═CH), 7.96 (s, 1H, C₄—H), 7.93 (d, 2H, Ar—H, J=8.8 Hz), 7.50 (d, 2H, Ar—H, J=9.0 Hz), 6.90 (d, 2H, Ar—H, J=9.0 Hz), 3.76-3.74 (m, 4H, morp-H), 3.05-3.04 (m, 4H, morp-H); LC-MS: 477.09 (M+1).

### Example 31

### (E)-4-((2-(N-(4-(4-Methylpiperazin-1-yl)phenyl)acetamido)-6-oxo-5H-pyrimido[4,5-b][1,4]thiazin-7(6H)-ylidene)methyl)benzoic acid (1l)

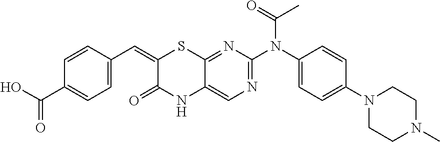 [SMILES: CC(=O)N(c1ccc(N2CCN(C)CC2)cc1)c1ncc2c(n1)S/C(=C/c1ccc(C(=O)O)cc1)C(=O)N2]

A solution of compound 4a (500 mg, 1.4 mmol), 4-carboxybenzaldehyde (210 mg, 1.4 mmol), triethylamine (780 μL, 5.6 mmol) and acetic anhydride (10 mL) were heated at 120° C. for 2 h. The solvent was removed and the crude compound was purified by column chromatography (Neutral alumina 150 mesh, 2-5% MeOH/CHCl₃) to get give compound 1l (120 mg); m.p.: 326-330° C.; ¹H NMR: δ 11.44 (brs, 1H, NH), 8.25 (s, 1H, ═CH), 8.06 (d, 2H, Ar—H, J=8.0 Hz), 7.95 (s, 1H, C₄—H), 7.78 (d, 2H, Ar—H, J=7.6 Hz), 7.07 (d, 2H, Ar—H, J=8.8 Hz), 6.94 (d, 2H, Ar—H, J=8.5 Hz), 3.18-3.16 (m, 4H, pip-H), 2.27-2.25 (m, 4H, pip-H), 2.11 (s, 3H, NCH₃), 1.93 (s, 3H, NCOCH₃); LC-MS: 531.14 (M+1).

### Example 32

### (E)-2-((4-(4-Methylpiperidin-1-yl)phenyl)amino)-7-(4-nitrobenzylidene)-5H-pyrimido[4,5-b][1,4]thiazin-6(7H)-one (1m)

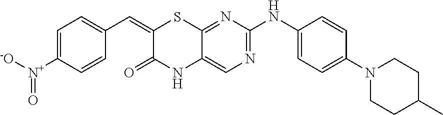

2-((4-(4-methylpiperidin-1-yl)phenyl)amino)-5H-pyrimido[4,5-b][1,4]thiazin-6(7H)-one (4g) (500 mg, 1.4 mmol) was condensed with 4-nitrobenzaldehyde (213 mg, 1.4 mmol) in the presence of triethylamine (780 μl, 5.6 mmol) in acetic anhydride (10 mL) at 120° C. The solvent is was removed and the crude product was purified by column chromatography (Basic alumina 50-200 μm, 2-5% MeOH/CHCl₃) afforded pure product (280 mg); m.p: 280-282° C.; ¹H NMR: δ 11.16 (brs, 1H, NH), 9.40 (brs, 1H, NH), 8.35 (d, 2H, Ar—H, J=8.8 Hz), 8.04 (s, 1H, —CH), 7.95 (s, 1H, C₄—H), 7.92 (d, 2H, Ar—H, J=8.8 Hz), 7.45 (d, 2H, Ar—H, J=9.0 Hz), 6.87 (d, 2H, Ar—H, J=8.9 Hz), 3.56-3.55 (m, 2H, pip-H), 2.59-2.55 (m, 2H, pip-H), 1.70-1.68 (m, 2H, pip-H), 1.48-1.46 (m, 1H, pip-H), 1.28-122 (m, 2H, pip-H), 0.95 (d, 3H, CH—CH₃, J=6.4 Hz); LC-MS: 489.16 (M+1).

### Example 33

### (E)-4-((2-((4-(4-Methylpiperazin-1-yl)phenyl)amino)-6-oxo-5H-pyrimido[4,5-b][1,4]thiazin-7(6H)-ylidene)methyl)benzonitrile (1n)

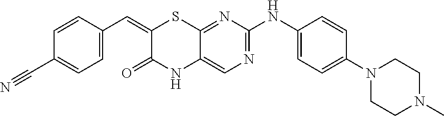 [SMILES: CN1CCN(c2ccc(Nc3ncc4c(n3)S/C(=C/c3ccc(C#N)cc3)C(=O)N4)cc2)CC1]

Condensation of 4-cyanobenzaldehyde (184 mg, 1.4 mmol) with compound 4a (500 mg, 1.4 mmol) in the presence of triethylamine ((780 μL, 5.6 mmol) in acetic anhydride (10 mL) followed by purification of the crude compound using column chromatography (Basic alumina 50-200 μm, 2-5% MeOH/CHCl₃) afforded pure compound 1n (230 mg); m.p.: 274-276° C.; ¹H NMR: δ 11.13 (brs, 1H, NH), 9.41 (brs, 1H, NH), 8.04 (s, 1H, ═CH), 7.98 (d, 2H, Ar—H, J=8.2 Hz), 7.91 (s, 1H, C₄—H), 7.84 (d, 2H, Ar—H, J=8.2 Hz), 7.47 (d, 2H, Ar—H, J=8.9 Hz), 6.88 (d, 2H, Ar—H, J=8.9 Hz), 3.07-3.06 (m, 4H, pip-H), 2.47-2.46 (m, 4H, pip-H), 2.23 (s, 3H, N—CH₃); LC-MS: 470.31 (M+1).

### Example 34

### (E)-7-(4-(Benzyloxy)benzylidene)-2-((4-(4-methylpiperazin-1-yl)phen y)amino)-5H-pyrimido[4,5-b][1,4]thiazin-6(7H)-one (1o)

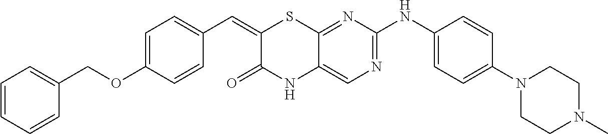 [SMILES: CN1CCN(c2ccc(Nc3ncc4c(n3)S/C(=C/c3ccc(OCc5ccccc5)cc3)C(=O)N4)cc2)CC1]

A solution of compound 4a (500 mg, 1.4 mmol), 4-benzyloxybenzaldehyde (297 mg, 1.4 mmol), triethylamine (780 μL, 5.6 mmol) and acetic anhydride (10 mL) was heated at 120° C. for 2 h. The solvent was removed and the crude compound was purified by column chromatography (Basic alumina 50-200 μm, 2-5% MeOH/CHCl₃) to give the pure compound 1o (290 mg); m.p.: 238-240° C.; ¹H NMR: δ 10.88 (brs, 1H, NH), 936 (brs, 1H, NH), 8.00 (s, 1H, ═CH), 7.82 (s, 1H, C₄—H), 7.65 (d, 2H, Ar—H, J=8.3 Hz), 7.48-7.47 (m, 4H, Ar—H), 7.44-7.42 (m, 2H, Ar—H), 7.38-7.37 (m, 1H, Ar—H), 7.18 (d, 2H, Ar—H, J=8.3 Hz), 6.89 (d, 2H, Ar—H, J=8.5 Hz), 5.20 (s, 2H, Ph-CH₂), 3.08-3.07 (m, 4H, pip-H), 2.47-2.46 (m, 4H, pip-H), 2.23 (s, 3H, NCH₃); LC-MS: 551.34 (M+1).

### Example 35

### (E)-4-((2-((4-(4-Methylpiperazin-1-yl)phenyl)amino)-6-oxo-5H-pyrimido[4,5-b][1,4]thiazin-7(6H)-ylidene)methyl)benzamide (1p)

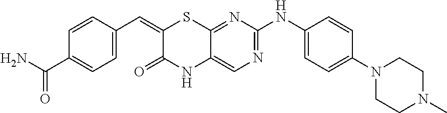 [SMILES: CN1CCN(c2ccc(Nc3ncc4c(n3)S/C(=C/c3ccc(C(N)=O)cc3)C(=O)N4)cc2)CC1]

Compound 4a (500 mg, 1.4 mmol) condensed with 4-formylbenzamide (209 mg, 1.4 mmol) in the presence of triethylamine (780 μL, 5.6 mmol) in acetic anhydride (10 mL) at 120° C. The solvent was removed and the crude product was purified by column chromatography (Basic alumina 50-200 μm, 2-5% MeOH/CHCl₃) afforded pure compound 1p (280 mg); m.p: 294-298° C.; ¹H NMR: δ 11.06 (brs, 1H, NH), 9.41 (brs, 1H, NH), 8.10 (s, 1H, ═CH), 8.04-8.00 (m, 3H, Ar—H), 7.92 (s, 1H, C₄—H), 7.73 (d, 2H, Ar—H, J=8.1 Hz), 7.48 (brs, 3H, CONH₂ & Ar—H), 6.90 (d, 2H, Ar—H, J=8.8 Hz), 3.08-3.07 (m, 4H, pip-H), 2.50-2.49 (m, 4H, pip-H), 2.26 (s, 3H, N—CH₃); LC-MS: 488.17 (M+1).

### Example 36

### (E)-7-(4-Hydroxybenzylidene)-2-((4-(4-methylpiperazin-1-yl)phenyl)amino)-5H-pyrimido[4,5-b][1,4]thiazin-6(7H)-one (1q)

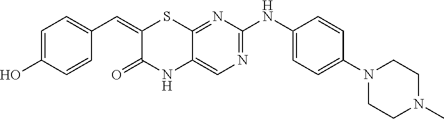 [SMILES: CN1CCN(c2ccc(Nc3ncc4c(n3)S/C(=C/c3ccc(O)cc3)C(=O)N4)cc2)CC1]

Anhydrous potassium carbonate (110 mg, 0.8 mmol) was added to a stirred solution of compound 1c (200 mg, 0.4 mmol) in methanol (10 mL) and N,N-dimethylformamide (5 mL) at room temperature and stirring was continued for 4 h. The mixture was poured in to crushed ice with stirring and stirring was continued for another 15 min. The separated solid was collected by filtration, then washed with water (10 mL) and ether (10 mL) and dried to give pure compound 1q (180 mg); m.p.: 312-314° C.; ¹H NMR: δ 10.83 (brs, 1H, NH), 10.11 (brs, 1H, Ar—OH), 9.36 (brs, 1H, NH), 7.99 (s, 1H, —CH), 7.79 (s, 1H, C₄—H), 7.55 (d, 2H, Ar—H, J=8.3 Hz), 7.49 (d, 2H, Ar—H, J=8.7 Hz), 6.92 (d, 2H, Ar—H, J=8.5 Hz), 6.90 (d, 2H, Ar—H, J=8.6 Hz), 3.07-3.06 (m, 4H, pip-H), 2.48-2.47 (m, 4H, pip-H), 2.25 (s, 3H, N—CH₃). LC-MS: 461.23 (M+1).

### Example 37

### Cytotoxicity of Selected Compounds on K562 and DU145 Cancer Cell Lines

The effect of the compounds described herein on tumor cells was determined by the assay described by Latham et al., Oncogene 12:827-837 (1996). Tumor cells K562 (chronic myelogenous leukemia; leukemia cell line +ve for Bcr-Abl) or DU145 (prostate cancer) were plated in 12-well dishes at a cell density of 2.5×10⁴ cells per well. The plated cells were treated 24h. later with a DMSO solution of a compound as described herein at multiple concentrations ranging from 0.01 μM to 100 μM. The plates were examined 96 h. later under an inverted microscope, Olympus CK-2 using a 10× objective, and compound activity was noted by physical observation. When necessary, the total number of viable cells was determined by trypsinizing the wells and counting the number of viable cells, as determined by trypan blue exclusion, using a hemacytometer. The IC₅₀ values for each compound are shown in Table 1.

TABLE 1

| Compound | K562^{†} | DU145^{†} |
|---|---|---|
| 4a | ++++ | +++ |
| 1a | ++++ | ++++ |
| 1a | ++++ | ++++ |
| 4d | ++ | ++ |
| 1b | +++++ | +++++ |
| 1c | ++++ | ++++ |
| 1d | ++++ | ++++ |
| 1e | +++ | ++++ |
| 1f | +++ | +++ |
| 1g | ++++ | ++++ |
| 1h | +++++ | ++++ |
| 1i | ++++ | ++++ |
| 1j | + | ++ |
| 1k | + | + |
| 1l | + | + |
| 1m | ++++ | ++++ |
| 1n | +++++ | +++++ |
| 1o | +++++ | +++++ |
| 1p | ++++ | ++++ |
| 1q | +++++ | +++++ |

^{†} IC₅₀ values are indicated as follows: +++++: IC₅₀: <1 μM ++++: IC₅₀: 1-10 μM +++: IC₅₀: >10-25 μM ++: IC₅₀: >25-50 μM +: IC₅₀: >50-100

### Example 38

### Kinase Inhibition Assay

Kinase assays were performed at Reaction Biology Corporation. To a freshly prepared buffer solution was added the target kinase at a concentration of 20 μM. The contents were mixed gently, and then compound 1b dissolved in DMSO was added to the reaction mixture in the appropriate concentration. The mixture was incubated at room temperature for 30 min. prior to addition of ATP to initiate the reaction. Compound 1b was tested in a 5-dose IC₅₀ mode with 10-fold serial dilutions starting at 10 μM. Staurosporine was used as a control compound in a 10-dose IC₅₀ mode with 3-fold serial dilutions starting at 20 μM. Reaction was carried out at 10 μM ATP concentration.

Results are shown in Table 2.

TABLE 2

| Kinase | IC₅₀ (nM) |
|---|---|
| ABL1 | 19 |
| ABL2/ARG | 30 |
| RPIK2 | 68 |
| FGR | 124 |
| PI3K-α | 13 |
| PI3K-δ | 20 |

### Example 39

### Inhibition of ABL Mutants

Kinase assays were performed at Reaction Biology Corporation. To a freshly prepared buffer solution was added the target kinase at a concentration of 20 μM. The contents were mixed gently, and then compound 1b dissolved in DMSO was added to the reaction mixture in the appropriate concentration. The mixture was incubated at room temperature for 30 min. prior to addition of ATP to initiate the reaction. Compound 1b was tested in a 5-dose IC₅₀ mode with 10-fold serial dilutions starting at 10 μM. Staurosporine was used as a control compound in a 10-dose IC₅₀ mode with 3-fold serial dilutions starting at 20 μM. Reaction was carried out at 10 μM ATP concentration.

Results are shown in Table 3.

TABLE 3

| Kinase | IC50 (nm) |
|---|---|
| ABL1 (E255K) | 106 |
| ABL1 (G250E) | 35 |
| ABL1 (H396P) | 9 |
| ABL1 (M351T) | 12 |
| ABL1 (Q252H) | 10 |
| ABL1 (T315I) | 2734 |
| ABL1 (Y253F) | 60 |

### Example 40

### Inhibition of PI3K Kinases

Kinase assays were performed at Reaction Biology Corporation. To a freshly prepared buffer solution was added the target kinase at a concentration of 20 μM. The contents were mixed gently and then compound 1b dissolved in DMSO was added to the reaction mixture in the appropriate concentration. The mixture was incubated at room temperature for 30 min. prior to addition of ATP to initiate the reaction. Compound 1b was tested in a 5-dose IC₅₀ mode with 10-fold serial dilutions starting at 10 μM. Staurosporine was used as a control compound in a 10-dose IC₅₀ mode with 3-fold serial dilutions starting at 20 M. Reaction was carried out at 10 μM ATP concentration.

Results are shown in Table 4.

TABLE 4

| Kinase | IC₅₀ (nM) |
|---|---|
| PIK3-α | 4 |
| PIK3-β | 10 |
| PIK3-γ | 22 |
| PIK3-δ | 7 |

### Example 41

### Cytotoxicity Assay

The following tumor cell lines were tested using a dose response end point assay system. The cells were grown in either DMEM or RPMI supplemented with 10% fetal bovine serum and 1 unit/mL Penicillin-Streptomycin solution. The tumor cells were plated into 6-well dishes at a cell density of 1.0×10⁵ cells/mL/well and compounds were added 24 h. later at various concentrations. Cell counts were determined from duplicate wells after 96 h. of treatment. The total number of viable cells was determined by trypan blue exclusion.

TABLE 5

| Cell Line | Tumor Type | GI50 Values (μM) |
|---|---|---|
| NAMALWA | Burkitt's Lymphoma | 0.17 |
| MV-4-11 | AML FLT3-ITD | 0.8 |
| K562 | CML | 0.15 |
| HL60 | AML: Promyelocytic | 1.0 |
| CEM | T-ALL | 0.16 |
| Z138C | Mantle Cell Lymphoma | 0.65 |
| MOLT-4 | T-ALL | 0.18 |
| U266B1 | Multiple Myeloma | 0.62 |
| GRANTA 519C | Mantle Cell Lymphoma | 0.15 |
| DAUDI | Burkitt's Lymphoma | 0.15 |

A number of embodiments of the invention have been described. Nevertheless, it will be understood that various modifications may be made without departing from the spirit and scope of the invention. Accordingly, other embodiments are within the scope of the following claims.

1. A compound of formula (I): 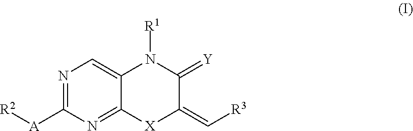
or a salt thereof, wherein:
A is selected from O, NR⁴ and S(O)ₘ;
R¹ is H or a substituted or unsubstituted (C₁-C₁₀)hydrocarbyl;
R² is selected from substituted or unsubstituted (C₁-C₁₀)hydrocarbyl and substituted or unsubstituted heterocyclyl;
R³ is selected from substituted or unsubstituted (C₆-C₁₀)aryl and substituted or unsubstituted (C₂-C₉)heteroaryl;
R⁴ is selected from H, (C₁-C₆)alkyl and —C(═O)R⁵;
or R² or R⁴ in combination with the nitrogen to which they are attached form a substituted or unsubstituted heterocyclyl;
R⁵ is selected from H and (C₁-C₆)alkyl;
X is S(O)ₙ;
Y is selected from O, S and NR⁶;
R⁶ is selected from H, —OH, (C₁-C₆)alkyl and —O—(C₁-C₆)alkyl;
m is an integer selected from 0, 1 and 2; and
n is an integer selected from 0, 1 and 2.

2. A compound according to claim 1, or a salt thereof, wherein:
R¹, when substituted, is substituted with one or more substituents independently selected from halogen, —OR^{a1}, (CH₂)_{q1}OR^{a1}, —SR^{a1}, —NO₂, —NR^{a1}R^{b1}, —CN, (C₁-C₆)hydrocarbyl, (C₁-C₆)haloalkyl, —C(═O)R^{a1}, —C(═O)OR^{a1}, —C(═O)NR^{a1}R^{b1}, —C(═NR^{a1})NR^{a1}₂, —OC(═O)R^{a1}, —OC(═O)OR^{a1}, —OC(═O)NR^{a1}₂, —O—(CH₂)_{q1}OR^{a1}, —O—(CH₂)_{q1}NR^{a1}R^{b1}, —O—(CH₂)_{q1}-halo, —NR^{a1}C(═O)R^{a1}, —NR^{a1}C(═O)OR^{a1}, —NR^{a1}C(═O)NR^{a1}₂, —NR^{a1}SO₂R^{a1}, —S(O)R^{a1}, —SO₂R^{a1}, —O—SO₃R^{a1}, —O—SO₂R^{a1}, —SO₂NR^{a1}₂, —O—P(═O)(OR^{1a})₂, —P(═O)(OR^{1a})₂, 4-methylpiperazin-1-yl, 4-BOC-piperazin-1-yl, 4-acetylpiperazin-1-yl, —O-glycoside and —O-glucuronide;
each q1 is an integer independently selected from 2, 3 and 4;
R^{a1} and R^{b1} are independently selected from H and (C₁-C₆)hydrocarbyl;
or R^{a1} and R^{b1} in any NR^{a1}R^{b1} group optionally together to which they are attached form a heterocyclic ring;
R², when substituted, is substituted with one or more substituents independently selected from R² halogen, —OR^{a2}, (CH₂)_{q2}OR^{a2}, —SR^{a2}, —NO₂, —NR^{a2}R^{b2}, —CN, (C₁-C₆)hydrocarbyl, (C₁-C₆)haloalkyl, —C(═O)R^{a2}, —C(═O)OR^{a2}, —C(═O)NR^{a2}R^{b2}, —C(═NR^{a2})NR^{a2}₂, —OC(═O)R^{a2}, —OC(═O)OR^{a2}, —OC(═O)NR^{a2}₂, —O—(CH₂)_{q2}OR^{a2}, —O—(CH₂)_{q2}NR^{a2}R^{b2}, —O—(CH₂)_{q2}-halo, —NR^{a2}C(═O)R^{a2}₂, —NR^{a2}C(═O)OR^{a2}, —NR^{a2}C(═O)NR^{a2}₂, —NR^{a2}SO₂R^{a2}, —S(O)R^{a2}, —SO₂R^{a2}, —O—SO₃R^{a2}, —O—SO₂R^{a2}, —SO₂NR^{a2}₂, —O—P(═O)(OR^{a2})₂, —P(═O)(OR^{a2})₂, 4-methylpiperazin-1-yl, 4-BOC-piperazin-1-yl, 4-acetylpiperazin-1-yl, —O-glycoside and —O-glucuronide;
wherein q2 is an integer independently selected from 2, 3 and 4;
R^{a2} and R^{b2} are independently selected from H and (C₁-C₆)hydrocarbyl;
or R^{a2} and R^{b2} in any NR^{a2}R^{b2} group optionally together to which they are attached form a heterocyclic ring:
R³ is unsubstituted aryl or heteroaryl or aryl or heteroaryl substituted with one or more substituents independently selected from (C₁-C₆)alkyl, (C₂-C₆)alkenyl, (C₂-C₆)alkynyl, halogen, (C₁-C₆)haloalkyl, —CN, —NO₂, —C(═O)R^{R3}, —OC(═O)Ar^{R3}, —C(═O)OR^{R3}, —C(═O)NR^{R3}₂, —C(═NR^{R3})NR^{R3}₂, —OR^{R3}, —Ar^{R3}, —OAr^{R3}, —((C₁-C₆)alkylene)Ar^{R3}, —O((C₁-C₆)alkylene)Ar^{R3}, —OC(═O)(C₁-C₆)alkyl, —OC(═O)O(C₁-C₆)alkyl, —OC(═O)NR^{R3}₂, —NR^{R3}₂, —NR^{R3}Ar^{R3}, —NR^{R3}((C₁-C₆)alkylene)Ar^{R3}, —NR^{R3}C(═O)R^{R3}, —NR^{R3}C(═O)Ar^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl, —(C₂-C₆)alkylene-OR^{R3}, —O(C₂-C₆)alkylene-N((C₁-C₆)alkyl)₂, —P(═O)(OR^{R3})₂ and —OP(═O)(OR^{R3})₂;
each R^{R3} is independently selected from H and (C₁-C₆)alkyl; and
each Ar^{R3} is unsubstituted aryl or heteroaryl or aryl or heteroaryl substituted with one or more of (C₁-C₆)alkyl, (C₁-C₆)alkenyl, (C₂-C₆)alkynyl, halogen, (C₁-C₆)haloalkyl, —CN, —NO₂, —C(═O)R^{R3}, —C(═O)OR^{R3}, —C(═O)NR^{R3}₂, —C(═NR^{R3})NR^{R3}₂, —OR^{R3}, —OC(═O)(C₁-C₆)alkyl, —OC(═O)O(C₁-C₆)alkyl, —OC(═O)NR^{R3}₂, —NR^{R3}₂, —NR^{R3}C(═O)R^{R3}, —NR^{R3}C(═O)(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl and —(C₂-C₆)allylene-OR^{R3}.

3. A compound according to claim 1, or a salt thereof, wherein R¹ is H or (C₁-C₆)alkyl.

4. A compound according to claim 1, or a salt thereof, wherein R¹ is H.

5-6. (canceled)

7. A compound according to claim 1, or a salt thereof, wherein R² is unsubstituted or substituted (C₆-C₁₀)aryl.

8-9. (canceled)

10. A compound according to claim 1, or a salt thereof, wherein R² is selected from 4-(4-acetylpiperazin-1-yl)phenyl, 4-(4-methylpiperazin-1-yl)phenyl, 4-(4-methylpiperidin-1-yl)phenyl and 4-morpholinophenyl.

11. A compound according to claim 1, or a salt thereof, wherein R² is a group according to the following formula: 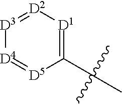
wherein:
D¹ is N or C-E¹;
D² is N or C-E²;
D³ is N or C-E³;
D⁴ is N or C-E⁴; and
D⁵ is N or C-E⁵;
provided that not more than three of D¹, D², D³, D⁴ and D⁵ is N;
E¹, E², E⁴ and E⁵ each independently selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(═O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂;
E³ is selected from H, (C₁-C₆)alkyl, (C₂-C₆)alkenyl, (C₂-C₆)alkynyl, halogen, —CN, —NO₂, —C(═O)R⁷, —C(═O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂; and a substituted or unsubstituted heterocyclic ring linked via a nitrogen atom; and
each R⁷ is independently selected from H and (C₁-C₆)alkyl.

12. A compound according to claim 11, or a salt thereof, wherein D³ is C-E³ and E³ is a group according to the formula —NR⁸R⁹; wherein:
R⁸ and R⁹ in combination form a group according to the formula —(C₁-C₃)alkylene-Q^{E3}-(C₁-C₃)alkylene,
Q^{E3} is selected from a bond, —CH₂—, —CH((C₁-C₆)alkyl)-, —C((C₁-C₆)alkyl)₂-, —CHAr^{E3}-, —C((C₁-C₆)alkyl)Ar^{E3}-, —O—, —S—, —NH—, —N((C₁-C₆)alkyl)-, —N(C(═O)(C₁-C₆)alkyl))-, —NAr^{E3}- and —NC(═O)Ar^{E3};
Ar^{E3} is an aryl or heteroaryl ring, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, —(C₁-C₆)haloalkyl, CN, —NO₂, —C(═O)R^{E3}, —C(═O)OR^{E3}, —C(═O)NR^{E3}₂, —C(═NR^{E3})NR^{E3}₂, —OR^{E3}, —OC(═O)(C₁-C₆)alkyl, —OC(═O)O(C₁-C₆)alkyl, —OC(═O)NR^{E3}₂, —NR^{E3}₂, —NR^{E3}C(═O)R^{E3}, —NR^{E3}C(═O)O(C₁-C₆)alkyl, —NR^{E3}C(═O)NR^{E3}₂, —NR^{E3}SO₂R^{E3}, —SR^{E3}, —S(O)R^{E3}, —SO₂R^{E3}, —OSO₂(C₁-C₆)alkyl, —SO₂NR^{E3}₂, (C₁-C₈)perfluoroalkyl, —(C₂-C₆)alkylene-OR^{E3}, —O(C₂-C₆)alkylene-N((C₁-C₆)alkyl)₂, —P(═O)(OR^{E3})₂ and —OP(═O)(OR^{E3}); and
each R^{E3} is independently H or (C₁-C₆)alkyl.

13. A compound according to claim 11, or a salt thereof, wherein none or one of D¹, D², D³, D⁴ and D⁵ is N.

14-27. (canceled)

28. A compound according to claim 1, or a salt thereof, wherein A is NR⁴.

29. A compound according to claim 28, or a salt thereof, wherein A is NH.

30-31. (canceled)

32. A compound according to claim 28, or a salt thereof, wherein R² and R⁴ together with the nitrogen to which they are attached form an unsubstituted or substituted heterocyclyl.

33. (canceled)

34. A compound according to claim 28, wherein:
R² and R⁴ together form a group according to the formula —(C₁-C₃)alkylene-Q^{R4}-(C₁-C₃)alkylene;
Q^{R4} is selected from a bond, —CH₂—, —CH((C₁-C₆)alkyl)-, —C((C₁-C₆)alkyl)₂-, —CHAr^{R4}—, —C((C₁-C₆)alkyl)Ar^{R4}—, —O—, —S—, —NH—, —N((C₁-C₆)alkyl)-, —NC(═O)(C₁-C₆)alkyl)-, —NAr^{R4}- and —NC(═O)Ar^{E3};
Ar^{R4} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{R4}, —C(═O)OR^{R4}, —C(═O)NR^{R4}₂, —C(═NR^{R4})NR^{R4}₂, —OR^{R4}, —OC(═O)(C₁-C₆)alkyl, —OC(═O)O(C₁-C₆)alkyl, —OC(═O)NR^{R4}₂, —NR^{R4}₂, —NR^{R4}C(═O)R^{R4}, —NR^{R4}C(═O)O(C₁-C₆)alkyl, —NR^{R4}C(═O)NR^{R4}₂, —NR^{R4}SO₂R^{R4}, —SR^{R4}, —S(O)R^{R4}, —SO₂R^{R4}, —OSO₂(C₁-C₆)alkyl, —SO₂NR^{R4}₂, (C₁-C₈)perfluoroalkyl, —(C₂-C₆)alkylene-OR^{R4}, —O(C₂-C₆)alkylene-N((C₁-C₆)alkyl)₂, —P(═O)(OR^{R4})₂ and —OP(═O)(OR^{R4})₂; and
each R^{R4} is independently selected from H and (C₁-C₆)alkyl.

35. A compound according to claim 34, or a salt thereof, wherein Q^{R4} is —NAr^{R4}-.

36. A compound according to claim 32, wherein R² and R⁴ together form a piperidine or piperazine ring.

37. (canceled)

38. A compound according to claim 32, or a salt thereof, wherein —NR²R⁴ is selected from 4-(pyridin-2-yl)piperazin-1-yl and 4-(pyrimidin-2-yl)piperazin-1-yl.

39. A compound according to claim 1, or a salt thereof, wherein R³ is substituted or unsubstituted aryl.

40. A compound according to claim 39, or a salt thereof, wherein R³ is unsubstituted aryl or aryl substituted with one or more substituents independently selected from (C₁-C₆)alkyl, (C₂-C₆)alkenyl, (C₂-C₆)alkynyl, halogen, (C₁-C₆)haloalkyl, —CN, —NO₂, —C(═O)R^{R3}, —OC(═O)Ar^{R3}, —C(═O)OR^{R3}, —C(═O)NR^{R3}₂, —C(═NR^{R3})NR^{R3}₂, —OR^{R3}, —Ar^{R3}, —OAr^{R3}, —((C₁-C₆)alkylene)Ar^{R3}, —O((C₁-C₆)alkylene)Ar^{R3}, —OC(═O)(C₁-C₆)alkyl, —OC(═O)O(C₁-C₆)alkyl, —OC(═O)NR^{R3}₂, —NR^{R3}₂, —NR^{R3}Ar^{R3}, —NR^{R3}((C₁-C₆)alkylene)Ar^{R3}, —NR^{R3}C(═O)R^{R3}, —NR^{R3}C(═O)Ar^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}, (C₁-C₈)perfluoroalkyl, —(C₂-C₆)alkylene-OR^{R3}, —O(C₂-C₆)alkylene-N((C₁-C₆)alkyl)₂, —P(═O)(OR^{R3})₂ and —OP(═O)(OR^{R3})₂; wherein each R^{R3} is independently selected from H and (C₁-C₆)alkyl; and wherein each Ar^{R3} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{R3}, —C(═O)OR^{R3}, —C(═O)NR^{R3}₂, —C(═NR^{R3})NR^{R3}₂, —OR^{R3}, —OC(═O)(C₁-C₆)alkyl, —OC(═O)O(C₁-C₆)alkyl, —OC(═O)NR^{R3}₂, —NR^{R3}₂, —NR^{R3}C(═O)R^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl and —(C₂-C₆)alkylene-OR^{R3}.

41. A compound according to claim 39, or a salt thereof, wherein R³ is substituted or unsubstituted phenyl.

42. A compound according to claim 1, or a salt thereof, wherein
R³ is selected from 4-acetoxyphenyl, 4-aminophenyl, 4-benzyloxyphenyl,
4-carboxyphenyl, 4-carbamoylphenyl, 4-cyanophenyl, 4-fluorophenyl, 4-hydroxyphenyl,
4-methoxy-3-nitrophenyl and 4-nitrophenyl and 1-acetyl-1H-indol-3-yl.

43. A compound according to claim 1, or a salt thereof, wherein R³ is substituted or unsubstituted heteroaryl.

44. (canceled)

45. A compound according to claim 1, or a salt thereof, wherein R³ is unsubstituted heteroaryl or heteroaryl substituted with one or more substituents independently selected from (C₁-C₆)alkyl, (C₂-C₆)alkenyl, (C₂-C₆)alkynyl, halogen, (C₁-C₆)haloalkyl, —CN, —NO₂, —C(═O)R^{R3}, —OC(═O)Ar^{R3}, —C(═O)OR^{R3}, —C(═O)NR^{R3}₂, —C(═NR^{R3})NR^{R3}₂, —OR^{R3}, —Ar^{R3}, —OAr^{R3}, —((C₁-C₆)alkylene)Ar^{R3}, —O((C₁-C₆)alkylene)Ar^{R3}, —OC(═O)(C₁-C₆)alkyl, —OC(═O)O(C₁-C₆)alkyl, —OC(═O)NR^{R3}₂, —NR^{R3}₂, —NR^{R3}Ar^{R3}, —NR^{R3}((C₁-C₆)alkylene)Ar^{R3}, —NR^{R3}C(═O)R^{R3}, —NR^{R3}C(═O)Ar^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl, —(C₂-C₆)alkylene-OR^{R3}, —O(C₂-C₆)alkylene-N((C₁-C₆)alkyl)₂, —P(═O)(OR^{R3})₂ and —OP(═O)(OR^{R3})₂; wherein each R^{R3} is independently selected from H and (C₁-C₆)alkyl; and wherein each Ar^{R3} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{R3}, —C(═O)OR^{R3}, —C(═O)NR^{R3}₂, —C(═NR^{R3})NR^{R3}₂, —OR^{R3}, —OC(═O)(C₁-C₆)alkyl, —OC(═O)O(C₁-C₆)alkyl, —OC(═O)NR^{R3}, —NR^{R3}₂, —NR^{R3}C(═O)R^{R3}, —NR^{R3}C(═O)O(C₁-C₆)alkyl, —NR^{R3}C(═O)NR^{R3}₂, —NR^{R3}SO₂R^{R3}, —SR^{R3}, —S(O)R^{R3}, —SO₂R^{R3}, —OSO₂(C₁-C₆)alkyl, —SO₂NR^{R3}₂, (C₁-C₈)perfluoroalkyl and —(C₂-C₆)alkylene-OR^{R3}.

46. A compound according to claim 1, or a salt thereof, wherein R³ is a group according to the following formula: 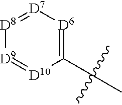
wherein;
D⁶ is N or C-E⁶;
D⁷ is N or C-E⁷;
D⁸ is N or C-E⁸;
D⁹ is N or C-E⁹; and
D¹⁰ is N or C-E¹⁰;
provided that not more than three of D¹, D², D³, D⁴ and D⁵ is N;
E⁶ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E6}, —OC(═O)Ar^{E6}, —C(═O)OR^{E6}, —C(═O)NR^{E6}₂, —O((C₁-C₆)alkylene)Ar^{E6}, —OC(═O)(C₁-C₆)alkyl, —OC(═O)O(C₁-C₆)alkyl, —OC(═O)NR^{E6}₂, —NR^{E6}₂, —NR^{E6}Ar^{E6}, —NR^{E6}((C₁-C₆)alkylene)Ar^{E6}, —NR^{E6}C(═O)R^{E6}, —NR^{E6}C(═O)Ar^{E6}, —NR^{E6}C(═O)O(C₁-C₆)alkyl, —NR^{E6}C(═O)NR^{E6}₂, —NR^{E6}SO₂R^{E6}, —SR^{E6}, —S(O)R^{E6}, —SO₂R^{E6}, —OSO₂(C₁-C₆)alkyl, —SO₂NR^{E6}₂, (C₁-C₈)perfluoroalkyl, —(C₂-C₆)alkylene-OR^{E6}, —O(C₂-C₆)alkylene-N((C₁-C₆)alkyl)₂, —P(═O)(OR^{E6})₂ and —OP(═O)(OR^{E6})₂;
each R^{E6} is independently selected from H and (C₁-C₆)alkyl;
each Ar^{E6} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E6}, —C(═O)OR^{E6}, —C(═O)NR^{E6}₂, —C(═NR^{E6})NR^{E6}₂, —OR^{E6}, —OC(═O)(C₁-C₆)alkyl, —C(═O)O(C₁-C₆)alkyl, —OC(═O)NR^{E6}₂, —NR^{E6}₂, —NR^{E6}C(═O)R^{E6}, —NR^{E6}C(═O)O(C₁-C₆)alkyl, —NR^{E6}C(═O)NR^{E6}₂, —NR^{E6}SO₂R^{E6}, —SR^{E6}, —S(O)R^{E6}, —SO₂R^{E6}, —OSO₂(C₁-C₆)alkyl, —SO₂NR^{E6}₂, (C₁-C₈)perfluoroalkyl and —(C₂-C₆)alkylene-OR^{E6};
E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R^{E7}, —NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N((C₁-C₆)alkyl)₂, —P(═O)(OR^{E7})₂ and —OP(═O)(OR^{E7})₂;
each R^{E7} is independently selected from H and (C₁-C₆)alkyl;
each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, CN, —NO₂, —C(═O)R^{E7}, —C(═O)OR^{E7}, —C(═O)NR^{E7}₂, —C(═NR^{E7})NR^{E7}₂, —OR^{E7}, —OC(═O)(C₁-C₆)alkyl, —C(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)allyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl and —(C₂-C₆)alkylene-OR^{E7};
E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, —NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl, —(C₂-C₆)alkylene-OR^{E8}, —O(C₂-C₆)alkylene-N((C₁-C₆)alkyl)₂, —P(═O)(OR^{E8})₂ and —OP(═O)(OR^{E8})₂;
each R^{E8} is independently selected from H and (C₁-C₆)alkyl;
each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E8}, —C(═O)OR^{E8}, —C(═O)NR^{E8}₂, —C(═NR^{E8})NR^{E8}₂, —OR^{E8}, —OC(═O)(C₁-C₆)alkyl, —OC(═O)O(C₁-C₆)alkyl, —OC(═O)NR^{E8}₂, —NR^{E8}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}₂, —SR^{E8}₂, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8};
E⁹ is selected from H, (C₁-C₆)alkyl, (C₂-C₆)alkenyl, (C₂-C₆)alkynyl, halogen, (C₁-C₆)haloalkyl, —CN, —NO₂, C(═O)R^{E9}, —OC(═O)Ar^{E9}, —C(═O)OR^{E9}, C(═O)NR^{E9}₂, —C(═NR^{E9})NR^{E9}₂, —OR^{E9}, —Ar^{E9}, —OAr^{E9}, —((C₁-C₆)alkylene)Ar^{E9}, —O((C₁-C₆)alkylene)Ar^{E9}, —OC(═O)(C₁-C₆)alkyl, —OC(═O)O(C₁-C₆)alkyl, —OC(═O)NR^{E9}₂, —NR^{E9}₂, —NR^{E9}Ar^{E9}, —NR^{E9}((C₁-C₆)alkylene)Ar^{E9}, —NR^{E9}C(═O)R^{E9}, —NR^{E9}C(═O)Ar^{E9}, —NR^{E9}C(═O)O(C₁-C₆)alkyl, —NR^{E9}C(═O)NR^{E9}₂, —NR^{E9}SO₂R^{E9}, —SR^{E9}, —S(O)R^{E9}, —SO₂R^{E9}, —OSO₂(C₁-C₆)alkyl, —SO₂NR^{E9}₂, (C₁-C₈)perfluoroalkyl, —(C₂-C₆)alkylene OR^{E9}, —O(C₂-C₆)alkylene-N((C₁-C₆)alkyl)₂, —P(═O)(OR^{E9})₂ and —OP(═O)(OR^{E9})₂;
each R^{E9} is independently selected from H and (C₁-C₆)alkyl;
each Ar^{E9} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, C(═O)R^{E9}, C(═O)OR^{E9}, C(═O)NR^{E9}₂, —C(═NR^{E9})NR^{E9}₂, —OR^{E9}, —OC(═O)(C₁-C₆)alkyl, —C(═O)O(C₁-C₆)alkyl, —OC(═O)NR^{E9}₂, —NR^{E9}₂, —NR^{E9}C(O)R^{E9}, —NR^{E9}C(═O)O(C₁-C₆)allyl, —NR^{E9}C(═O)NR^{E9}₂, —NR^{E9}SO₂R^{E9}, —SR^{E9}, —S(O)R^{E9}, —SO₂R^{E9}, —OSO₂(C₁-C₆)alkyl, —SO₂NR^{E9}₂, (C₁-C₈)perfluoroalkyl and —(C₂-C₆)alkylene-OR^{E9};
E¹⁰ is selected from H, (C₁-C₆)alkyl, (C₂-C₆)alkenyl, (C₂-C₆)alkynyl, halogen, (C₁-C₆)haloalkyl, —CN, —NO₂, C(═O)R^{E10}, —O(═O)Ar^{E10}, —C(═O)OR^{E10}, —C(═O)NR^{E10}₂, C(═NR^{E10})NR^{E10}₂, —OR^{E10}, Ar^{E10}, —OAr^{E10}, —((C₁-C₆)alkylene)Ar^{E10}, —O((C₁-C₆)alkylene)Ar^{E10}, —OC(═O)(C₁-C₆)alkyl, —OC(═O)O(C₁-C₆)alkyl, —OC(═O)NR^{E10}₂, —NR^{E10}₂, —NR^{E10}Ar^{E10}, —NR^{E10}((C₁-C₆)alkylene)Ar^{E10}, —NR^{E10}C(═O)R^{E10}, —NR^{E10}C(═O)Ar^{E10}, —NR^{E10}C(═O)O(C₁-C₆)alkyl, —NR^{E10}C(═O)NR^{E10}₂, —NR^{E10}SO₂R^{E10}, —SR^{E10}, —S(O)R^{E10}, —SO₂R^{E10}, —OSO₂(C₁-C₆)alkyl, —SO₂NR^{E10}₂, (C₁-C₈)perfluoroalkyl, (C₂-C₆)alkylene-OR^{E10}, —O(C₂-C₆)alkylene-N((C₁-C₆)alkyl)₂, —P(═O)(OR^{E10})₂ and —OP(═O)(OR^{E10});
each R^{E10} is independently selected from H and (C₁-C₆)alkyl; and
each Ar^{E10} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, C(═O)R^{E10}, C(═O)OR^{E10}, —C(═O)NR^{E10}₂, C(═NR^{E10})NR^{E10}₂, —OR^{E10}, —OC(═O)(C₁-C₆)alkyl, —OC(═O)O(C₁-C₆)alkyl, —OC(═O)NR^{E10}₂, —NR^{E10}₂, —NR^{E10}C(═O)R^{E10}, —NR^{E10}C(═O)O(C₁-C₆)alkyl, —NR^{E10}C(═O)NR^{E10}₂, —NR^{E10}SO₂R^{E10}, —SR^{E10}, —S(O)R^{E10}, —SO₂R^{E10}, —OSO₂(C₁-C₆)alkyl, —SO₂NR^{E10}₂, (C₁-C₈)perfluoroalkyl and —(C₂-C₆)alkylene-R^{E10}.

47-62. (canceled)

63. A compound according to claim 1, or a salt thereof, wherein X is S.

64. A compound according to claim 1, or a salt thereof, wherein Y is O.

65. A compound according to claim 1, of formula (I-A): 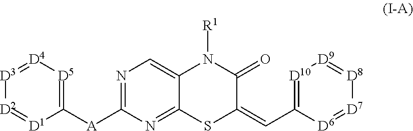
or a salt thereof, wherein:
A is selected from O, NR⁴ and S(O)ₘ;
R¹ is H or (C₁-C₆)alkyl;
D¹ is N or C-E¹;
D² is N or C-E²;
D³ is N or C-E³;
D⁴ is N or C-E⁴;
D⁵ is N or C-E⁵;
D⁶ is N or C-E⁶;
D⁷ is N or C-E⁷;
D⁸ is N or C-E⁸;
D⁹ is N or C-E⁹; and
D¹⁰ is N or C-E¹⁰;
provided that not more than three of D¹, D², D³, D⁴ and D⁵ is N; and
provided that not more than three of D⁶, D⁷, D^{S}, D⁹ and D¹⁰ is N;
E¹, E², E⁴ and E⁵ each independently selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂;
E³ is selected from H, (C₁-C₆)alkyl, (C₂-C₆)alkenyl, (C₂-C₆)alkynyl, halogen, (C₁-C₆)haloalkyl, —N, —NO₂, —C(═O)R⁷, —C(═O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂; and a substituted or unsubstituted 5 heterocyclic ring linked via a nitrogen atom;
each R⁷ is independently selected from H and (C₁-C₆)alkyl;
E⁶ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E6}, —OC(═O)Ar^{E6}, —C(═O)OR^{E6}, —C(═O)NR^{E6}₂, —O((C₁-C₆)alkylene)Ar^{E6}, —OC(═O)(C₁-C₆)alkyl, —OC(═O)O(C₁-C₆)alkyl, —OC(═O)NR^{E6}₂, —NR^{E6}₂, —NR^{E6}Ar^{E6}, —NR^{E6}((C₁-C₆)alkylene)Ar^{E6}, —NR^{E6}C(═O)R^{E6}, —NR^{E6}C(═O)Ar^{E6}, —NR^{E6}C(═O)O(C₁-C₆)alkyl, —NR^{E6}C(═O)NR₂, —NR^{E6}SO₂R^{E6}, —SR^{E6}, —S(O)R^{E6}, —SO₂R^{E6}, —OSO₂(C₁-C₆)alkyl, —SO₂NR₂, (C₁-C₈)perfluoroalkyl, —(C₂-C₆)alkylene OR^{E6}, —O(C₂-C₆)alkylene-N((C₁-C₆)alkyl)₂, —P(═O)(OR^{E6})₂ and —OP(═O)(OR^{E6})₂;
each R^{E6} is independently selected from H and (C₁-C₆)alkyl;
each Ar^{E6} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E6}, —C(═O)OR^{E6}, —C(═O)NR^{E6}₂, —C(═NR)NR^{E6}₂, —OR^{E6}, —OC(═O)(C₁-C₆)alkyl, —C(═O)O(C₁-C₆)alkyl, —OC(═O)NR^{E6}₂, —NR^{E6}₂, —NR^{E6}C(═O)R^{E6}, —NR^{E6}C(═O)O(C₁-C₆)alkyl, —NR^{E6}C(═O)NR^{E6}₂, —NR^{E6}SO₂R^{E6}, —SR^{E6}, —S(O)R^{E6}, —SO₂R^{E6}, —OSO₂(C₁-C₆)alkyl, —SO₂NR^{E6}₂, (C₁-C₈)perfluoroalkyl and —(C₂-C₆)alkylene OR^{E6};
E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R^{E7}, —NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N((C₁-C₆)alkyl)₂, —P(═O)(OR^{E7})₂ and —OP(═O)(OR^{E7})₂;
each R^{E7} is independently selected from H and (C₁-C₆)alkyl;
each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E7}, —C(═O)OR^{E7}, —C(═O)NR^{E7}₂, —C(═NR^{E7})NR^{E7}₂, —OR^{E7}, —OC(═O)(C₁-C₆)alkyl, —C(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl and —(C₂-C₆)alkylene-OR^{E7};
E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, —NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl, —(C₂-C₆)alkylene OR^{E8}, —O(C₂-C₆)alkylene-N((C₁-C₆)alkyl)₂, —P(═O)(OR^{E8})₂ and —OP(═O)(OR^{E8})₂;
each R^{E8} is independently selected from H and (C₁-C₆)alkyl;
each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E8}, —C(═O)OR^{E8}, —C(═O)NR^{E8}₂, —C(═NR^{E8})NR^{E8}₂, —OR^{E8}, —OC(═O)(C₁-C₆)alkyl, —C(═O)O(C₁-C₆)alkyl, —OC(═O)NR^{E8}₂, —NR^{E8}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene OR^{E8};
E⁹ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E9}, OC(═O)Ar^{E9}, —C(═O)OR^{E9}, —C(═O)NR^{E9}₂, —C(═NR^{E9})NR^{E9}₂, —OR^{E9}, —Ar^{E9}, —OAr^{E9}, —((C₁-C₆)alkylene)Ar^{E9}, —O((C₁-C₆)alkylene)Ar^{E9}, —OC(═O)(C₁-C₆)alkyl, —OC(═O)O(C₁-C₆)alkyl, —OC(═O)NR^{E9}₂, —NR^{E9}₂, —NR^{E9}Ar^{E9}, —NR^{E9}((C₁-C₆)alkylene)Ar^{E9}, —NR^{E9}C(═O)R^{E9}, —NR^{E9}C(═O)Ar^{E9}, —NR^{E9}C(═O)O(C₁-C₆)alkyl, —NR^{E9}C(═O)NR^{E9}₂, —NR^{E9}SO₂R^{E9}, —SR^{E9}, —S(O)R^{E9}, —SO₂R^{E9}, —OSO₂(C₁-C₆)alkyl, —SO₂NR^{E9}₂, (C₁-C₈)perfluoroalkyl, —(C₂-C₆)alkylene-OR^{E9}, —O(C₂-C₆)alkylene-N((C₁-C₆)alkyl)₂, —P(═O)(OR^{E9})₂ and —OP(═O)(OR^{E9})₂;
each R^{E9} is independently selected from H and (C₁-C₆)alkyl;
each Ar^{E9} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E9}, —C(═O)OR^{E9}, —C(═O)NR^{E9}₂, —C(═NR^{E9})NR^{E9}₂, —OR^{E9}, —OC(═O)(C₁-C₆)alkyl, —C(═O)O(C₁-C₆)alkyl, —OC(═O)NR^{E9}₂, —NR^{E9}₂, —NR^{E9}C(═O)R^{E9}, —NR^{E9}C(═O)O(C₁-C₆)alkyl, —NR^{E9}C(═O)NR^{E9}₂, —NR^{E9}SO₂R^{E9}, —SR^{E9}, —S(O)R^{E9}, —SO₂R^{E9}, —OSO₂(C₁-C₆)alkyl, —SO₂NR^{E9}₂, (C₁-C₈)perfluoroalkyl and —(C₂-C₆)alkylene-OR^{E9};
E¹⁰ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E10}, —OC(═O)Ar^{E10}, —C(═O)OR^{E10}, —C(═O)NR^{E10}₂, —C(═NR^{E10})NR^{E10}₂, —OR^{E10}, —Ar^{E10}, —OAr^{E10}, —((C₁-C₆)alkylene)Ar^{E10}, —O((C₁-C₆)alkylene)Ar^{E10}, —OC(═O)(C₁-C₆)alkyl, —OC(═O)O(C₁-C₆)alkyl, —OC(═O)NR^{E10}₂, —NR^{E10}₂, —NR^{E10}Ar^{E10}, —NR^{E10}((C₁-C₆)alkylene)Ar^{E10}, —NR^{E10}C(═O)R^{E10}, —NR^{E10}C(═O)Ar^{E10}, —NR^{E10}C(═O)O(C₁-C₆)alkyl, —NR^{E10}C(═O)NR^{E10}₂, —NR^{E10}SO₂R^{E10}, —SR^{E10}, —S(O)R^{E10}, —SO₂R^{E10}, —OSO₂(C₁-C₆)alkyl, —SO₂NR^{E10}₂, (C₁-C₈)perfluoroalkyl, —(C₂-C₆)alkylene-OR^{E10}, —O(C₂-C₆)alkylene-N((C₁-C₆)alkyl)₂, —P(═O)(OR^{E10})₂ and —P(═O)(OR^{E10})₂;
each R^{E10} is independently selected from H and (C₁-C₆)alkyl; and
each Ar^{E10} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E10}, —C(═O)OR^{E10}, —C(═O)NR^{E10}₂, —C(═NR^{E10})NR^{E10}₂, —OR^{E10}, —OC(═O)(C₁-C₆)alkyl, —OC(═O)O(C₁-C₆)alkyl, —OC(═O)NR^{E10}₂, —NR^{E10}₂, —NR^{E10}C(═O)R^{E10}, —NR^{E10}C(═O)O(C₁-C₆)alkyl, —NR^{E10}C(═O)NR^{E10}₂, —NR^{E10}SO₂R^{E10}, —SR^{E10}, —S(O)R^{E10}, —SO₂R^{E10}, —OSO₂(C₁-C₆)alkyl, —SO₂NR^{E10}₂, (C₁-C₈)perfluoroalkyl and —(C₂-C₆)alkylene-OR^{E10}.

66. A compound according to claim 65, or a salt thereof, wherein D¹ is C-E¹; D² is C-E²; D³ is C-E³; D⁴ is C-E⁴; D⁵ is C-E⁵; D⁶ is C-E⁶; D⁷ is C-E⁷; D^{S} is C-E⁸; D⁹ is C-E⁹;
and D¹⁰ is C-E¹⁰.

67-68. (canceled)

69. A compound according to claim 1 of formula (I-B): 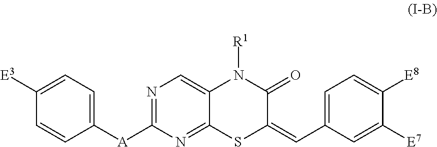
or a salt thereof, wherein:
A is selected from O, NR⁴ and S(O)ₘ;
R¹ is H or (C₁-C₆)alkyl;
E³ is selected from H, (C₁-C₆)alkyl, (C₂-C₆)alkenyl, (C₂-C₆)alkynyl, halogen, (C₁-C₆)haloalkyl, —CN, —NO₂, —C(═O)R⁷, —C(═O)OR⁷, —C(═O)NR⁷₂, —C(═NR⁷)NR⁷₂, —OR⁷, —OC(═O)(C₁-C₆)alkyl, —OC(═O)O(C₁-C₆)alkyl, —OC(═O)NR⁷₂, —NR⁷₂, —NR⁷C(═O)R⁷, —NR⁷C(═O)O(C₁-C₆)alkyl, —NR⁷C(═O)NR⁷₂, —NR⁷SO₂R⁷, —SR⁷, —S(O)R⁷, —SO₂R⁷, —OSO₂(C₁-C₆)alkyl, —SO₂NR⁷₂, (C₁-C₈)perfluoroalkyl, —(C₂-C₆)alkylene-OR⁷, —O(C₂-C₆)alkylene-N((C₁-C₆)alkyl)₂, —P(═O)(OR⁷)₂ and —OP(═O)(OR⁷)₂; and a substituted or unsubstituted heterocyclic ring linked via a nitrogen atom;
each R⁷ is independently selected from H and (C₁-C₆)alkyl;
E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R E⁷, —NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene OR^{E7}, —O(C₂-C₆)alkylene-N((C₁-C₆)alkyl)₂, —P(═O)(OR^{E7})₂ and —OP(═O)(OR^{E7})₂;
each R^{E7} is independently selected from H and (C₁-C₆)alkyl;
each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E7}, —C(═O)OR^{E7}, —C(═O)NR^{E7}₂, —C(═NR^{E7})NR^{E7}₂, —OR^{E7}, —OC(═O)(C₁-C₆)alkyl, —OC(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl and —(C₂-C₆)alkylene-OR^{E7};
E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, —NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR₂, (C₁-C₈)perfluoroalkyl, —(C₂-C₆)alkylene-OR^{E8}, —O(C₂-C₆)alkylene-N((C₁-C₆)alkyl)₂, —P(═O)(OR)₂ and —OP(═O)(OR^{E8})₂;
each R^{E8} is independently selected from H and (C₁-C₆)alkyl; and
each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E8}, —C(═O)OR^{E8}, —C(═O)NR^{E8}₂, —C(═NR)NR^{E8}₂, —OR^{E8}, —OC(═O)(C₁-C₆)alkyl, —OC(═O)O(C₁-C₆)alkyl, —OC(═O)NR^{E8}₂, —NR^{E8}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8}.

70. (canceled)

71. A compound according to claim 65, or a salt thereof, wherein E³ is a group according to the formula —NR⁸R⁹ wherein:
R⁸ and R⁹ in combination form a group according to the formula —(C₁-C₃)alkylene-Q^{E3}-(C₁-C₃)alkylene-;
Q^{E3} is selected from a bond, —CH₂—, —CH((C₁-C₆)alkyl)-, —C((C₁-C₆)alkyl)₂-, —CHAr^{E3}-, —C((C₁-C₆)alkyl)Ar^{E3}-, —O—, —S—, —NH—, —N((C₁-C₆)alkyl)-, —N(C(═O)(C₁-C₆)alkyl))-, —NAr^{E3}- and —NC(═O)Ar^{E3}-;
Ar^{E3} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{E3}, —C(═O)OR^{E3}, —C(═O)NR^{E3}₂, —C(═NR^{E3})NR^{E3}₂, —OR^{E3}, —OC(═O)(C₁-C₆)alkyl, —OC(═O)O(C₁-C₆)alkyl, —OC(═O)NR^{E3}₂, —NR^{E3}₂, —NR^{E3}C(═O)R^{E3}, —NR^{E3}C(═O)O(C₁-C₆)alkyl, —NR^{E3}C(═O)NR^{E3}₂, —NR^{E3}SO₂R^{E3}, —SR^{E3}, —S(O)R^{E3}, —SO₂R^{E3}, —OSO₂(C₁-C₆)alkyl, —SO₂NR^{E3}₂, (C₁-C₈)perfluoroalkyl, —(C₂-C₆)alkylene-OR^{E3}, —O(C₂-C₆)alkylene-N((C₁-C₆)alkyl)₂, —P(═O)(OR^{E3})₂ and —OP(═O)(OR^{E3}); and
each R^{E3} is independently H or (C₁-C₆)alkyl.

72. A compound according to claim 65, or a salt thereof, wherein E³ forms a piperazine ring.

73. A compound according to claim 1, or a salt thereof, wherein the compound is of formula (I-C) 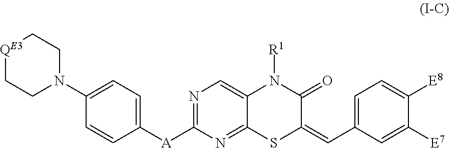
or a salt thereof, wherein:
A is selected from O, NR⁴ and S(O)ₘ;
R¹ is H or (C₁-C₆)alkyl;
Q^{E3} is selected from a bond, —CH₂—, —CH((C₁-C₆alkyl)-, —C((C₁-C₆)alkyl)₂-, —CHAr^{E3}-, —C((C₁-C₆)alkyl)Ar^{E3}-, —O—, —S—, —NH—, —N((C₁-C₆)alkyl)-, —N(C(═O)(C₁-C₆)alkyl))-, —NAr^{E3}- and —NC(═O)Ar^{E3};
Ar^{E3} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{E3}, —C(═O)OR^{E3}, —C(═O)NR^{E3}₂, —C(═NR^{E3})NR^{E3}₂, —OR^{E3}, —OC(═O)(C₁-C₆)alkyl, —OC(═O)O(C₁-C₆)alkyl, —OC(═O)NR^{E3}₂, —NR^{E3}₂, —NR^{E3}C(═O)R^{E3}, —NR^{E3}C(═O)O(C₁-C₆)alkyl, —NR^{E3}C(═O)NR^{E3}₂, —NR^{E3}SO₂R^{E8}, —SR^{E3}, —S(O)R^{E3}, —SO₂R^{E3}, —OSO₂(C₁-C₆)alkyl, —SO₂NR^{E3}₂, (C₁-C₈)perfluoroalkyl, —(C₂-C₆)alkylene-OR^{E3}, —O(C₂-C₆)alkylene-N((C₁-C₆)alkyl)₂, —P(═O)(OR^{E3})₂ and —OP(═O)(OR^{E3})₂; wherein each R^{E3} is independently H or (C₁-C₆)alkyl;
E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene) Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R^{E7}, —NR^{E7}C(═O)Ar^{E7}, —NR^{E7}C(═O)O(C₁-C₆)alkyl, —NR^{E7}C(═O)NR^{E7}₂, —NR^{E7}SO₁R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N(C₁-C₆)alkyl)₂, —P(═O)(OR^{E7})₂ and —OP(═O)OR^{E7})₂;
each R^{E7} is independently selected from H and (C₁-C₆)alkyl;
each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO, C(═O)R^{E7}, C(═O)OR^{E7}, C(═O)NR^{E7}, —C(═NR^{E7})NR^{E7}, —OR^{E7}, —OC(═O)(C₁-C₆)alkyl, —OC(═O)O(C₁-C₆)alkyl, —OC(═O)NR^{E7}₂, —NR^{E7}, —NR^{E7}C(═O)R^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₇₂NR^{E7}₂, (C₁-C₈)perfluoroalkyl and —(C₂-C₆)alkylene-OR^{E7}; and
E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, —NR^{E8}₂, —NR^{E8}Ar^{E8}, —NR^{E8}((C₁-C₈)alkylene)Ar^{E8}, —NR^{E8}C(═O)R^{E8}, —NR^{E8}C(═O)Ar^{E8}, —NR^{E8}C(═O)(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl, —(C₂-C₆)alkylene-OR^{E8}, —O(C₂-C₆)alkylene-N((C₁-C₆)alkyl)₂, —P(═O)(OR^{E8})₂ and —OP(═O)(OR^{E8})₂;
each R^{E8} is independently selected from H and (C₁-C₆)alkyl; and
each Ar^{E8} is unsubstituted aryl or heteroaryl or aryl or heteroaryl substituted with one or more of (C₁-C₆)alkyl, (C₁-C₆)alkenyl, (C₂-C₆)alkynyl, halogen, (C₁-C₆)haloalkyl, —CN, —NO₂, —C(═O)R^{E8}, —C(═O)OR^{E8}, —C(═O)NR^{E8}, —C(═NR^{E8})NR, —OR^{E8}, —OC(═O)(C₁-C₆)alkyl, —OC(═O)O(C₁-C₆)alkyl, —OC(═O)NR^{E8}₂, —NR^{E8}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8}.

74. A compound according to claim 65, or a salt thereof, wherein A is NR⁴.

75. A compound according to claim 74, or a salt thereof, wherein A is NH.

76. A compound according to claim 65, or a salt thereof, wherein A is O.

77. A compound according to claim 1 of formula (I-D): 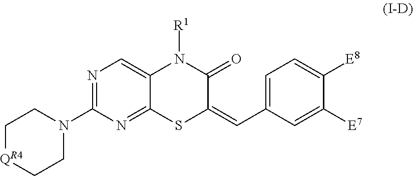
or a salt thereof, wherein:
R¹ is H or (C₁-C₆)alkyl;
Q^{R4} is selected from a bond, —CH₂—, —CH((C₁-C₆)alkyl)-, —C((C₁-C₆)alkyl)₂-, —CHAr^{R4}—, —C((C₁-C₆)alkyl)Ar^{R4}—, —O—, —S—, —NH—, —N((C₁-C₆)alkyl)-, —NC(═O)((C₁-C₆)alkyl)-, —NAr^{R4}— and —NC(═O)Ar^{R4};
Ar^{R4} is an aryl or heteroaryl, which is unsubstituted or optionally substituted with 1, 2, 3, 4, or 5 substituents, each independently selected from (C₁-C₆)alkyl, (C₂-C₆)alkenyl, (C₂-C₆)alkynyl, halogen, (C₁-C₆)haloalkyl, —CN, —NO₂, —C(═O)R^{R4}, —C(═O)OR^{R4}, —C(═O)NR^{R4}₂, —C(═NR^{R4})NR^{R4}₂, —OR^{R4}, —OC(═O)(C₁-C₆)alkyl, —OC(═O)O(C₁-C₆)alkyl, —OC(═O)NR^{R4}₂, —NR^{R4}₂, —NR^{R4}C(═O)R^{R4}, —NR^{R4}C(═O)O(C₁-C₆)alkyl, —NR^{R4}C(═O)NR^{R4}₂, —NR^{R4}SO₂R^{R4}, —SR^{R4}, —S(O)R^{R4}, —SO₂R^{R4}, —OSO₂(C₁-C₆)alkyl, —SO₂NR^{R4}₂, (C₁-C₈)perfluoroalkyl, —(C₂-C₆)alkylene-OR^{R4}, —O(C₂-C₆)alkylene-N((C₁-C₆)alkyl)₂, —P(═O)(OR^{R4})₂ and —OP(═O)(OR^{R4})₂;
each R^{R4} is independently selected from H and (C₁-C₆)alkyl;
E⁷ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —C(═NR^{E7})NR^{E7}₂, —OR^{E7}, —Ar^{E7}, —OAr^{E7}, —((C₁-C₆)alkylene)Ar^{E7}, —O((C₁-C₆)alkylene)Ar^{E7}, —OC(═O)(C₁-C₆)alkyl, —OC(═O)O(C₁-C₆)alkyl, —OC(═O)NR^{E7}₂, —NR^{E7}₂, —NR^{E7}Ar^{E7}, —NR^{E7}((C₁-C₆)alkylene)Ar^{E7}, —NR^{E7}C(═O)R^{E7}, —NR^{E7}C(═O)Ar^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl, —(C₂-C₆)alkylene-OR^{E7}, —O(C₂-C₆)alkylene-N((C₁-C₆)alkyl)₂, —P(═O)(OR^{E7})₂ and —OP(═O)(OR^{E7})₂;
each R^{E7} is independently selected from H and (C₁-C₆)alkyl;
each Ar^{E7} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)R^{E7}, —C(═O)OR^{E7}, —C(═O)NR^{E7}₂, —C(═NR^{E7})NR^{E7}₂, —OR^{E7}, —OC(═O)(C₁-C₆)alkyl, —OC(═O)O(C₁-C₆)alkyl, —OC(═O)NR^{E7}₂, —NR^{E7}₂, —NR^{E7}C(═O)R^{E7}, —NR^{E7}C(═O)O(C₁-C₆)alkyl, —NR^{E7}C(═O)NR^{E7}₂, —NR^{E7}SO₂R^{E7}, —SR^{E7}, —S(O)R^{E7}, —SO₂R^{E7}, —OSO₂(C₁-C₆)alkyl, —SO₂NR^{E7}₂, (C₁-C₈)perfluoroalkyl and —(C₂-C₆)alkylene-OR^{E7}; and
E⁸ is selected from H, (C₁-C₆)alkyl, (C₂-C₆)alkenyl, (C₂-C₆)alkynyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —C(═NR^{E8})NR^{E8}₂, —OR^{E8}, —Ar^{E8}, —OAr^{E8}, —((C₁-C₆)alkylene)Ar^{E8}, —O((C₁-C₆)alkylene)Ar^{E8}, —OC(═O)(C₁-C₆)alkyl, —OC(═O)O(C₁-C₆)alkyl, —OC(═O)NR^{E8}₂, —NR^{E8}₂, —NR^{E8}Ar^{E8}, —NR^{E8}((C₁-C₆)alkylene)Ar^{E8}, —NR^{E8}C(═O)R^{E8}, —NR^{E8}C(═O)Ar^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl, —(C₂-C₆)alkylene-OR^{E8}, —O(C₂-C₆)alkylene-N((C₁-C₆)alkyl)₂, —P(═O)(OR^{E8})₂ and —OP(═O)(OR^{E8})₂;
each R^{E8} is independently selected from H and (C₁-C₆)alkyl; and
each Ar^{E8} is unsubstituted aryl or heteroaryl or aryl or heteroaryl substituted with one or more of (C₁-C₆)alkyl, (C₂-C₆)alkenyl, (C₂-C₆)alkynyl, halogen, (C₁-C₆)haloalkyl, —CN, —NO₂, —C(═O)RE, —C(═O)OR^{E8}, —C(═O)NR^{E8}₂, —C(═NR^{E8})NR^{E8}₂, —OR^{E8}, —OC(═O)(C₁-C₆)alkyl, —OC(═O)O(C₁-C₆)alkyl, —OC(═O)NR^{E8}₂, —NR^{E8}₂, —NR^{E8}C(═O)R^{E8}, —NR^{E8}C(═O)O(C₁-C₆)alkyl, —NR^{E8}C(═O)NR^{E8}₂, —NR^{E8}SO₂R^{E8}, —SR^{E8}, —S(O)R^{E8}, —SO₂R^{E8}, —OSO₂(C₁-C₆)alkyl, —SO₂NR^{E8}₂, (C₁-C₈)perfluoroalkyl and —(C₂-C₆)alkylene-OR^{E8}.

78. A compound according to claim 77, or a salt thereof, wherein Q^{R4} is —NAr^{R4}— or Q^{R4} is —CHAr^{R4}—.

79. (canceled)

80. A compound according to claim 78, or a salt thereof, wherein Ar^{R4} is a pyridyl or pyrimidinyl ring.

81. A compound according to claim 65, or a salt thereof, wherein E⁷ is H, (C₁-C₆)alkyl, halogen, (C₁-C₆)haloalkyl, —CN, —NO₂, —C(═O)R^{E7}, —OC(═O)Ar^{E7}, —C(═O)OR^{E7}, —C(═O)NR^{E7}₂, —OR^{E7}, —Ar^{E7}, —OAr^{E7}, —O((C₁-C₆)alkylene)Ar^{E7}, —OC(═O)(C₁-C₆)alkyl, —NR^{E7}₂, —NR^{E7}Ar^{E7}, —NR^{E7}((C₁-C₆)alkylene)Ar^{E7}, —NR^{E7}C(═O)R^{E7}, or —NR^{E7}C(═O)Ar^{E7}.

82. A compound according to claim 81, or a salt thereof, wherein E⁷ is H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph,

83. A compound according to claim 81, or a salt thereof, wherein E⁷ is H or —NO₂.

84. A compound according to claim 65, or a salt thereof, wherein E⁸ is H, (C₁-C₆)alkyl, halogen, (C₁-C₆)haloalkyl, —CN, —NO₂, —C(═O)R^{E8}, —OC(═O)Ar^{E8}, —C(═O)OR^{E8}, —C(═O)NR^{E8}₂, —OR^{E8}, —Ar^{E8}, —OAr^{E8}, —O((C₁-C₆)alkylene)Ar^{E8}, —OC(═O)(C₁-C₆)alkyl, —NR^{E8}₂, —NR^{E8}Ar^{E8}, —NR^{E8}((C₁-C₆)alkylene)Ar^{E8}, —NR^{E8}C(═O)R^{E8}, or —NR^{E8}C(═O)Ar^{E8}.

85. A compound according to claim 84, or a salt thereof, wherein E⁸ is H, methyl, ethyl, —F, —Cl, —CN, —NO₂, —C(═O)Me, —OC(═O)Ph, —C(═O)OH, —C(═O)OMe, —C(═O)OEt, —C(═O)NH₂, —OH, —OMe, —OEt, -Ph, —OPh, —OCH₂Ph, —OCH₂CH₂Ph, —OC(═O)Me, —NH₂, —NHMe₂, —NMe₂, —NHPh, —NHCH₂Ph, —NMeCH₂Ph, —NHC(═O)Me, —NMeC(═O)Me, —NHC(═O)Ph, or —NMeC(═O)Ph,

86. A compound according to claim 65, or a salt thereof, wherein R¹ is H.

87. A compound according to claim 1, wherein the compound of formula (I) is selected from selected from compounds of the following formulae, and salts thereof:
(E)-7-(4-fluorobenzylidene)-2-(4-(4-methylpiperazin-1-yl)phenylamino)-5H-pyrimido[4,5-b][1,4]thiazin-6(7H)-one;
(E)-2-(4-(4-methylpiperazin-1-yl)phenylamino)-7-(4-nitrobenzylidene)-5H-pyrimido[4,5-b][1,4]thiazin-6(7H)-one;
(E)-4-((2-(4-(4-methylpiperazin-1-yl)phenylamino)-6-oxo-5H-pyrimido[4,5-b][1,4]thiazin-7(6N)-ylidene)methyl)phenyl acetate;
(E)-N-(7-(4-methoxy-3-nitrobenzylidene)-6-oxo-6,7-dihydro-5H-pyrimido[4,5-b][1,4]thiazin-2-yl)-N-(4-(4-methylpiperazin-1-yl)phenyl)acetamide;
(E)-7-(4-nitrobenzylidene)-2-(4-(pyridin-2-yl)piperazin-1-yl)-5H-pyrimido[4,5-b][1,4]thiazin-6(7N)-one;
(E)-2-(4-(4-acetylpiperazin-1-yl)phenoxy)-7-(4-nitrobenzylidene)-5H-pyrimido[4,5-b][1,4]thiazin-6(7H)-one;
(E)-N-(7-((1-acetyl-1H-indol-3-yl)methylene)-6-oxo-6,7-dihydro-5H-pyrimido[4,5-b][1,4]thiazin-2-yl)-N-(4-(4-methylpiperazin-1-yl)phenyl)acetamide;
(E)-7-(4-aminobenzlidene)-2-(4-(4-methylpiperazin-1-yl)phenylamino)-5H-pyrimido[4,5-b][1,4]thiazin-6(7H)-one;
(E)-4-((6-oxo-2-(4-(pyridin-2-yl)piperazin-1-yl)-5H-pyrimido[4,5-b][1,4]thiazin-7(6H)-ylidene)methyl)phenyl acetate;
(E)-4-((6-oxo-2-(4-(pyridin-2-yl)piperidin-1-yl)-5H-pyrimido[4,5-b][1,4]thiazin-7(6H)-ylidene)methyl)phenyl acetate;
(E)-7-(4-nitrobenzylidene)-2-(4-(pyrimidin-2-yl)piperazin-1-yl)-5H-pyrimido[4,5-b][1,4]thiazin-6(7H)-one;
(E)-2-((4-morpholinophenyl)amino)-7-(4-nitrobenzylidene)-5H-pyrimido[4,5-b][1,4]thiazin-6(7N)-one;
(E)-4-((2-(N-(4-(4-methylpiperazin-1-yl)phenyl)acetamido)-6-oxo-5H-pyrimido[4,5-b][1,4]thiazin-7(6H)-ylidene)methyl)benzoic acid;
(E)-2-((4-(4-methylpiperidin-1-yl)phenyl)amino)-7-(4-nitrobenzylidene)-5H-pyrimido[4,5-b][1,4]thiazin-6(7H)-one;
(E)-4-((2-((4-(4-methylpiperazin-1-yl)phenyl)amino)-6-oxo-5H-pyrimido[4,5-b][1,4]thiazin-7(6N)-ylidene)methyl)benzonitrile;
(E)-7-(4-(benzyloxy)benzylidene)-2-((4-(4-methylpiperazin-1-yl)phenyl)amino)-5H-pyrimido[4,5-b][1,4]thiazin-6(7H)-one;
(E)-4-((2-((4-(4-methylpiperazin-1-yl)phenyl)amino)-6-oxo-5H-pyrimido[4,5-b][1,4]thiazin-7(6H)-ylidene)methyl)benzamide; and
(E)-7-(4-hydroxybenzylidene)-2-((4-(4-methylpiperazin-1-yl)phenyl)amino)-5H-pyrimido[4,5-b][1,4]thiazin-6(7H)-one.

88-121. (canceled)

122. A pharmaceutical composition comprising a compound according to claim 1, or a pharmaceutically acceptable salt thereof, and a pharmaceutically acceptable carrier.

123. A compound of formula (II), (III), or (IV): 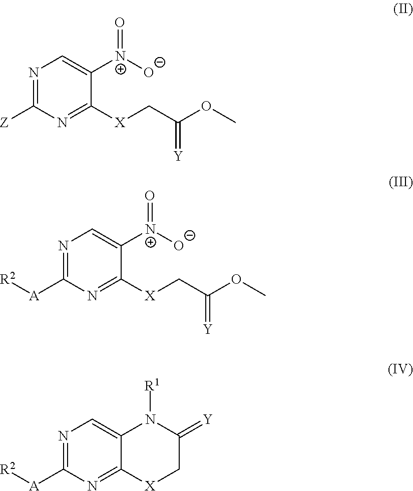
or a salt thereof, wherein:
R¹ is H or a substituted or unsubstituted (C₁-C₁₀)hydrocarbyl;
A is selected from O, NR⁴ and S(O)ₘ;
R² is selected from substituted or unsubstituted (C₁-C₁₀)hydrocarbyl and substituted or unsubstituted heterocyclyl;
R⁴ is selected from H, (C₁-C₆)alkyl and —C(═O)R⁵;
or R² or R⁴ in combination with the nitrogen to which they are attached form a substituted or unsubstituted heterocyclyl;
R⁵ is selected from H and (C₁-C₆)alkyl;
X is S(O)ₙ;
Y is selected from O, S and NR⁶;
Z is a halogen;
R⁶ is selected from hydrogen, —OH, (C₁-C₆)alkyl and —O—(C₁-C₆)alkyl;
m is an integer selected from 0.1 and 2; and
n is an integer selected from 0, 1 and 2.

124-126. (canceled)

127. A compound according to claim 123, or a salt thereof, wherein the compound is selected from the following compounds and salts thereof:
methyl 2-(2-chloro-5-nitropyrimidin-4-ylthio)acetate;
methyl 2-(2-(4-(4-methylpiperazin-1-yl)phenylamino)-5-nitropyrimidin-4-ylthio)acetate;
methyl 2-(5-nitro-2-(4-(pyridin-2-yl)piperazin-1-yl)pyrimidin-4-ylthio)acetate;
methyl 2-(5-nitro-2-(4-(piperazin-1-yl)phenoxy)pyrimidin-4-ylthio)acetate;
methyl 2-(2-(4-chlorophenylthio)-5-nitropyrimidin-4-ylthio acetate;
methyl 2-(2-(2,6-dichlorobenzylthio)-5-nitropyrimidin-4-ylthio)acetate;
methyl 2-((5-nitro-2-(4-(pyrimidin-2-yl)piperazin-1-yl)pyrimidin-4-yl)thio)acetate;
methyl 2-((2-((4-morpholinophenyl)amino)-5-nitropyrimidin-4-yl)thio)acetate; and
methyl 2-((2-((4-(4-methylpiperidin-1-yl)phenyl)amino)-5-nitropyrimidin-4-yl)thio)acetate;
2-(4-(4-methylpiperazin-1-yl)phenylamino)-5H-pyrimido[4,5-b][1,4]thiazin-5(7H)-one;
2-(4-(pyridin-2-yl)piperazin-1-yl-5H-pyrimido[4,5-b][1,4]thiazin-6(7H)-one;
2-(4-(piperazin-1-yl)phenoxy)-5H-pyrimido[4,5-b][1,4]thiazin-6(7H)-one;
2-(2,6-dichlorobenzylthio)-5H-pyrimido[4,5-b][1,4]thiazin-6(7H)one;
2-(4-(pyrimidin-2-yl)piperazin-1-yl)-5H-pyrimido[4,5-b][1,4]thiazin-6(7H)-one;
2-((4-Morpholinophenyl)amino)-5H-pyrimido[4,5-b][1,4]thiazin-6(7H)-one; and
2-((4-(4-Methylpiperidin-1-yl)phenyl)amino)-5H-pyrimido[4,5-b][1,4]thiazin-6(7H)-one.

128-146. (canceled)

147. A method for treating a cellular proliferative disorder in a patient, the method comprising administering to the patient a therapeutically effective amount of a compound according to claim 1, or a pharmaceutically acceptable salt thereof.

148-154. (canceled)

155. A method of treating a neurological disorder in a patient, the method comprising administering a therapeutically effective amount of a compound according to claim 1, or a pharmaceutically acceptable salt thereof.

156-168. (canceled)

169. A method of making a compound according to claim 1 of formula (I): 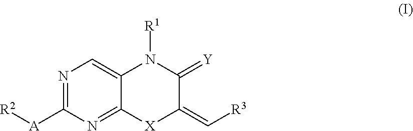
or a salt thereof, wherein A, R¹, R², R³, X and Y are as defined in claim 1, the method comprising reacting a compound of formula (IV): 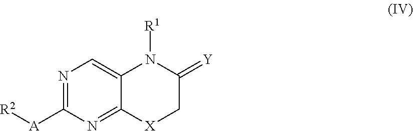
or a salt thereof, with a compound of formula (VI):
R³—C(═O)H (VI)
or a salt thereof, to form the compound of formula (I).

170. (canceled)
